(19) United States
(12) Patent Application Publication
(10) Pub. No.: US 2010/0184767 A1
(43) Pub. Date: Jul. 22, 2010

(54) SUBSTITUTED OXAZOLIDINONES AND USE THEREOF

(76) Inventor: Susanne Rohrig, Hilden (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(21) Appl. No.: 12/665,727

(22) PCT Filed: Jun. 7, 2008
(86) PCT No.: PCT/EP08/04564

§ 371 Date: Dec. 20, 2009

(30) Foreign Application Priority Data
Jun. 20, 2007 (DE) 10 2007 028 320.4

(51) Int. Cl. A61K 31/5377; C07D 413/14; A61K 31/454; A61K 31/513; A61P 29/00; A61P 31/04; A61P 11/00; A61P 7/02

(52) U.S. Cl. 514/236.8; 544/138; 546/209; 514/326; 544/316; 514/256

(57) ABSTRACT

The invention relates to novel substituted oxazolidinones, to processes for their preparation, to their use for the treatment and/or prophylaxis of diseases and their use for preparing medicaments for the treatment and/or prophylaxis of diseases, in particular of thromboembolic disorders.

The invention relates to novel substituted oxazolidinones, to processes for their preparation, to their use for the treatment and/or prophylaxis of diseases and their use for preparing medicaments for the treatment and/or prophylaxis of diseases, in particular of thromboembolic disorders.

Blood coagulation is a protective mechanism of the organism which helps to “seal” defects in the wall of the blood vessels quickly and reliably. Thus, loss of blood can be avoided or kept to a minimum. Haemostasis after injury of the blood vessels is effected mainly by the coagulation system in which an enzymatic cascade of complex reactions of plasma proteins is triggered. Numerous blood coagulation factors are involved in this process, each of which factors converts, on activation, the respectively next inactive precursor into its active form. At the end of the cascade comes the conversion of soluble fibrinogen into insoluble fibrin, resulting in the formation of a blood clot. In blood coagulation, traditionally the intrinsic and the extrinsic system, which end in a joint reaction path, are distinguished. Here, factor Xa, which is formed from the proenzyme factor X, has a key role, since it joins both coagulation paths. The activated serine protease Xa cleaves prothrombin to thrombin. For its part, the thrombin formed cleaves fibrinogen to fibrin. Subsequent crosslinking of the fibrin monomers results in the formation of blood clots and thus haemostasis. In addition, thrombin is a potent trigger of platelet aggregation, which also contributes considerably to haemostasis.

Haemostasis is subject to a complex regulatory mechanism. Uncontrolled activation of the coagulation system or defective inhibition of the activation processes may lead to the formation of local thromboses or embolisms in vessels (arteries, veins, lymph vessels) or cardiac cavities. This may lead to serious thromboembolic disorders. In addition, hypercoagulability may—systemically—in the case of consumption coagulopathy lead to disseminated intravasal coagulation. Thromboembolic complications are furthermore encountered in microangiopathic haemolytic anaemias, extracorporeal circulatory systems, such as haemodialysis, and also prosthetic heart valves.

Thromboembolic disorders are the most frequent cause of morbidity and mortality in most industrialized countries [Heart Disease: A Textbook of Cardiovascular Medicine, Eugene Braunwald, 5th edition, 1997, W.B. Saunders Company, Philadelphia].

The anticoagulants known from the prior art, i.e. substances for inhibiting or preventing blood coagulation, have various, frequently grave disadvantages. Accordingly, in practice, efficient treatment methods or the prophylaxis of thromboembolic disorders is found to be very difficult and unsatisfactory.

For the therapy and prophylaxis of thromboembolic disorders, use is firstly made of heparin which is administered parenterally or subcutaneously. By virtue of more favourable pharmacokinetic properties, these days preference is increasingly given to low-molecular-weight heparin; however, this does likewise not avoid the known disadvantages, described hereinbelow, of heparin therapy. Thus, heparin is orally ineffective and has only a comparatively short half-life. Since heparin inhibits several factors of the blood coagulation cascade simultaneously, the action is unselective. In addition, there is a high risk of bleeding, where in particular cerebral bleeding and bleeding in the gastrointestinal tract may occur, and there may be thrombopenia, alopecia medicomentosa or osteoporosis [Pschyrembel, Klinisches Wörterbuch [Clinical Dictionary], 257th edition, 1994, Walter de Gruyter Verlag, page 610, key word “heparin”; Römpp Lexikon Chemie [Römpp Chemical Encyclopaedia], Version 1.5, 1998, Georg Thieme Verlag Stuttgart, key word “heparin”].

A second class of anticoagulants are the vitamin K antagonists. These include, for example, 1,3-indanediones, but especially compounds such as warfarin, phenprocoumone, dicoumarole and other coumarin derivatives which unselectively inhibit the synthesis of various products of certain vitamin-K-dependent coagulation factors in the liver. However, owing to the mechanism of action, the onset of activity is very slow (latency time to onset of action 36 to 48 hours). The compounds can be administered orally; however, owing to the high risk of bleeding and the narrow therapeutic index, complicated individual adjustment and observation of the patient is required [J. Hirsh, J. Dalen, D. R. Anderson et al., “Oral anticoagulants: Mechanism of action, clinical effectiveness, and optimal therapeutic range” Chest 2001, 119, 8S-21S; J. Ansell, J. Hirsh, J. Dalen et al., “Managing oral anticoagulant therapy” Chest 2001, 119, 22S-38S; P. S. Wells, A. M. Holbrook, N. R. Crowther et al., “Interactions of warfarin with drugs and food” Ann. Intern. Med. 1994, 121, 676-683].

Recently, a new therapeutic approach for treatment and prophylaxis of thromboembolic disorders has been described. The aim of this novel therapeutic approach is the inhibition of factor Xa. In accordance with the central roll which factor Xa plays in the blood coagulation cascade, factor Xa is one of the most important targets for anticoagulatory active compounds [J. Hauptmann, J. Stürzebecher, Thrombosis Research 1999, 93, 203; S. A. V. Raghavan, M. Dikshit, “Recent advances in the status and targets of antithrombotic agents” Drugs Fut. 2002, 27, 669-683; H. A. Wieland, V. Laux, D. Kozian, M. Lorenz, “Approaches in anticoagulation: Rationales for target positioning” Curr. Opin. Investig. Drugs 2003, 4, 264-271; U. J. Ries, W. Wienen, “Serine proteases as targets for antithrombotic therapy” Drugs Fut. 2003, 28, 355-370; L.-A. Linkins, J. I. Weitz, “New anticoagulant therapy” Annu. Rev. Med. 2005, 56, 63-77; A. Casimiro-Garcia et al., “Progress in the discovery of Factor Xa inhibitors” Expert Opin. Ther. Patents 2006, 15, 119-145].

Here, it has been shown that various compounds, both peptidic and non-peptidic, are effective as factor Xa inhibitors in animal models. To date, a large number of direct factor Xa inhibitors is known [J. M. Walenga, W. P. Jeske, D. Hoppensteadt, J. Fareed, “Factor Xa Inhibitors: Today and beyond” Curr. Opin. Investig. Drugs 2003, 4, 272-281; J. Ruef, H. A. Katus, “New antithrombotic drugs on the horizon” Expert Opin. Investig. Drugs 2003, 12, 781-797; M. L. Quan, J. M. Smallheer, “The race to an orally active Factor Xa inhibitor: Recent advances” Curr. Opin. Drug Discovery & Development 2004, 7, 460-469]. Oxazolidinones as non-peptidic, low-molecular-weight factor Xa inhibitors are described in WO 01/47919.

For antithrombotic medicaments, the therapeutic width is of central importance: the difference between the therapeutically active dose for coagulation inhibition and the dose where bleeding may occur should be as big as possible so that maximum therapeutic activity is achieved at a minimum risk profile. The therapeutic width of an antithrombotically active compound depends on the changes in the plasma levels of an active compound during the course of the day after the administration of the medicament. The peak-to-trough ratio, i.e. the ratio between the maximum level after administration of the medicament and the minimum level at the end of the treatment interval may be used as a measure for this. For an optimum oral antithrombotic medicament, this peak-to-trough ratio should be as small as possible, so that the occurrence of bleeding by reduced maximum levels can be avoided and that sufficiently high minimum levels ensure antithrombotic activity during the entire treatment interval.

Accordingly, it is an object of the present invention to provide novel alternative compounds having a comparable or better activity and a broad therapeutic window for controlling diseases, in particular thromboembolic disorders, in humans and animals.

The invention provides compounds of the formula

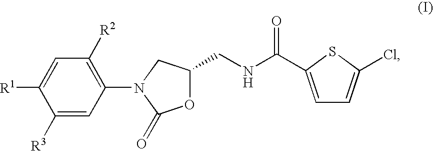

in which
R¹ represents a group of the formula

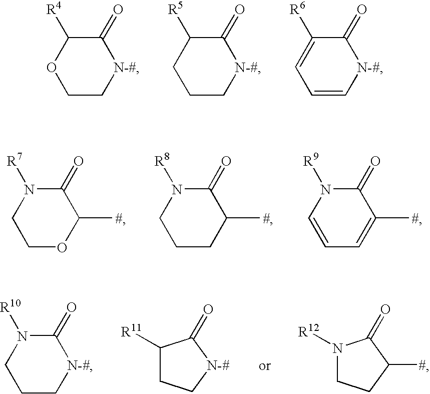

where
# is the point of attachment to the phenyl ring,
R⁴ represents hydrogen or C₁-C₃-alkyl,
where alkyl may be substituted by a substituent, where the substituent is selected from the group consisting of hydroxyl, C₁-C₃-alkoxy and C₃-C₆-cycloalkyloxy,
R⁵ represents hydrogen, hydroxyl, C₁-C₃-alkyl, C₁-C₃-alkoxy or C₃-C₆-cycloalkyloxy, where alkyl and alkoxy may be substituted by a substituent, where the substituent is selected from the group consisting of hydroxyl, C₁-C₃-alkoxy and C₃-C₆-cycloalkyloxy,
R⁶ represents hydrogen, hydroxyl, C₁-C₃-alkyl, C₁-C₃-alkoxy or C₃-C₆-cycloalkyloxy, where alkyl and alkoxy may be substituted by a substituent, where the substituent is selected from the group consisting of hydroxyl, C₁-C₃-alkoxy and C₃-C₆-cycloalkyloxy,
R⁷ represents hydrogen, C₁-C₃-alkyl or C₃-C₆-cycloalkyl,
where C₂-C₃-alkyl may be substituted by a substituent, where the substituent is selected from the group consisting of hydroxyl, C₁-C₃-alkoxy and C₃-C₆-cycloalkyloxy,
R⁸ represents hydrogen, C₁-C₃-alkyl or C₃-C₆-cycloalkyl,
where C₂-C₃-alkyl may be substituted by a substituent, where the substituent is selected from the group consisting of hydroxyl, C₁-C₃-alkoxy and C₃-C₆-cycloalkyloxy,
R⁹ represents hydrogen, C₁-C₃-alkyl or C₃-C₆-cycloalkyl,
where C₂-C₃-alkyl may be substituted by a substituent, where the substituent is selected from the group consisting of hydroxyl, C₁-C₃-alkoxy and C₃-C₆-cycloalkyloxy,
R¹⁰ represents hydrogen, C₁-C₃-alkyl or C₃-C₆-cycloalkyl,
where C₂-C₃-alkyl may be substituted by a substituent, where the substituent is selected from the group consisting of hydroxyl, C₁-C₃-alkoxy and C₃-C₆-cycloalkyloxy,
R¹¹ represents hydrogen, hydroxyl, C₁-C₃-alkyl, C₁-C₃-alkoxy or C₃-C₆-cycloalkyloxy, where alkyl and alkoxy may be substituted by a substituent, where the substituent is selected from the group consisting of hydroxyl, C₁-C₃-alkoxy and C₃-C₆-cycloalkyloxy,
R¹² represents hydrogen, C₁-C₃-alkyl or C₃-C₆-cycloalkyl,
where C₂-C₃-alkyl may be substituted by a substituent, where the substituent is selected from the group consisting of hydroxyl, C₁-C₃-alkoxy and C₃-C₆-cycloalkyloxy,
R² represents fluorine, chlorine, cyano, trifluoromethyl or trifluoromethoxy,
R³ represents hydrogen, chlorine, methyl, ethyl, n-propyl, methoxy, ethoxy or methoxymethyl,
and their salts, their solvates and the solvates of their salts.

Compounds according to the invention are the compounds of the formula (I) and their salts, solvates and solvates of the salts, the compounds, comprised by formula (I), of the formulae mentioned below and their salts, solvates and solvates of the salts and the compounds, comprised by the formula (I), mentioned below as exemplary embodiments and their salts, solvates and solvates of the salts if the compounds, comprised by formula (I), mentioned below are not already salts, solvates and solvates of the salts.

Depending on their structure, the compounds according to the invention can exist in stereoisomeric forms (enantiomers, diastereomers). Accordingly, the invention comprises the enantiomers or diastereomers and their respective mixtures. From such mixtures of enantiomers and/or diastereomers, it is possible to isolate the stereoisomerically uniform components in a known manner.

If the compounds according to the invention can be present in tautomeric forms, the present invention comprises all tautomeric forms.

In the context of the present invention, preferred salts are physiologically acceptable salts of the compounds according to the invention. The invention also comprises salts which for their part are not suitable for pharmaceutical applications, but which can be used, for example, for isolating or purifying the compounds according to the invention.

Physiologically acceptable salts of the compounds according to the invention include acid addition salts of mineral acids, carboxylic acids and sulfonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalene disulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of customary bases, such as, by way of example and by way of preference, alkali metal salts (for example sodium salts and potassium salts), alkaline earth metal salts (for example calcium salts and magnesium salts) and ammonium salts, derived from ammonia or organic amines having 1 to 16 carbon atoms, such as, by way of example and by way of preference, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

In the context of the invention, solvates are those forms of the compounds according to the invention which, in solid or liquid state, form a complex by coordination with solvent molecules. Hydrates are a specific form of the solvates where the coordination is with water. In the context of the present invention, preferred solvates are hydrates.

Moreover, the present invention also comprises prodrugs of the compounds according to the invention. The term “prodrugs” includes compounds which for their part may be biologically active or inactive but which, during the time they spend in the body, are converted into compounds according to the invention (for example metabolically or hydrolytically).

In the context of the present invention, the substituents have, unless specified otherwise, the following meaning:

alkyl per se and “alk” and “alkyl” in alkoxy represent a straight-chain alkyl radical having generally 1 to 3, preferably 1 or 2, carbon atoms, by way of example and by way of preference methyl, ethyl and n-propyl.
alkoxy represents, by way of example and by way of preference, methoxy, ethoxy and n-propoxy.
cycloalkyl represents a cycloalkyl group having generally 3 to 6 carbon atoms, preferably 3 to 5 carbon atoms, by way of example and by way of preference cyclopropyl, cyclobutyl, cyclopentyl and cyclohexyl.
cycloalkyloxy represents a cycloalkyloxy group having generally 3 to 6 carbon atoms, preferably 3 to 5 carbon atoms, by way of example and by way of preference cyclopropyloxy, cyclobutyloxy, cyclopentyloxy and cyclohexyloxy.

In the formulae of the group which may represent R¹, the end point of the line which is marked by a # is not a carbon atom or a CH₂ group, but is part of the bond to the atom to which R¹ is attached.

A symbol * at a carbon atom means that the compound is present in enantiomerically pure form with respect to the configuration at this carbon atom, which is, in the context of the present invention, to be understood as meaning an enantiomeric excess of more than 90% (>90% ee).

Preference is given to compounds of the formula (I) in which

R¹ represents a group of the formula

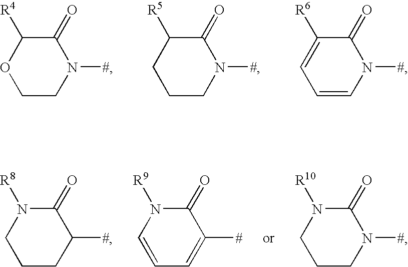

where
# is the point of attachment to the phenyl ring,
R⁴ represents hydrogen,
R⁵ represents hydrogen, hydroxyl, C₁-C₃-alkyl or C₁-C₃-alkoxy,
where alkyl and alkoxy may be substituted by a substituent, where the substituent is selected from the group consisting of hydroxyl and C₁-C₃-alkoxy,
R⁶ represents hydrogen, C₁-C₃-alkyl or C₁-C₃-alkoxy,
where alkyl and alkoxy may be substituted by a substituent, where the substituent is selected from the group consisting of hydroxyl and C₁-C₃-alkoxy,
R⁸ represents hydrogen, C₁-C₃-alkyl or C₃-C₆-cycloalkyl,
where C₂-C₃-alkyl may be substituted by a substituent, where the substituent is selected from the group consisting of hydroxyl and C₁-C₃-alkoxy,
R⁹ represents hydrogen, C₁-C₃-alkyl or C₃-C₆-cycloalkyl,
where C₂-C₃-alkyl may be substituted by a substituent, where the substituent is selected from the group consisting of hydroxyl and C₁-C₃-alkoxy,
R¹⁰ represents hydrogen, C₁-C₃-alkyl or C₃-C₆-cycloalkyl,
where C₂-C₃-alkyl may be substituted by a substituent, where the substituent is selected from the group consisting of hydroxyl and C₁-C₃-alkoxy,
R² represents fluorine or chlorine,
R³ represents hydrogen, methyl or methoxymethyl,
and their salts, their solvates and the solvates of their salts.

Preference is also given to compounds of the formula (I) in which

R¹ represents a group of the formula

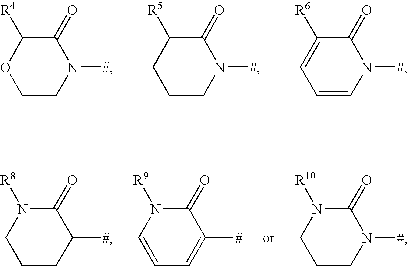

where
# is the point of attachment to the phenyl ring,
R⁴ represents hydrogen,
R⁵ represents hydrogen, hydroxyl or hydroxymethyl,
R⁶ represents hydrogen, methyl, hydroxymethyl, 2-hydroxyeth-1-yl or 2-hydroxyeth-1-oxy,
R⁸ represents hydrogen or methyl,
R⁹ represents hydrogen or methyl,
R¹⁰ represents methyl, ethyl or 2-hydroxyeth-1-yl,
R² represents fluorine or chlorine,
R³ represents hydrogen or methyl,
and their salts, their solvates and the solvates of their salts.

Preference is also given to compounds of the formula (I) in which

R¹ represents a group of the formula

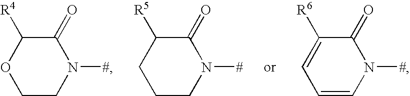

where
# is the point of attachment to the phenyl ring,
R⁴ is hydrogen,
R⁵ is hydrogen, hydroxyl or hydroxymethyl,
R⁶ is hydroxymethyl or 2-hydroxyeth-1-oxy,
R² is fluorine or chlorine,
R³ is hydrogen or methyl,
and their salts, their solvates and the solvates of their salts.

Preference is also given to compounds of the formula (I) in which R¹ represents a group of the formula

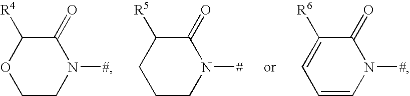

where
# is the point of attachment to the phenyl ring,
R⁴ represents hydrogen,
R⁵ represents hydrogen, hydroxyl or hydroxymethyl,
R⁶ represents hydroxymethyl, 2-hydroxyeth-1-yl or 2-hydroxyeth-1-oxy.

Preference is also given to compounds of the formula (I) in which R¹ represents a group of the formula

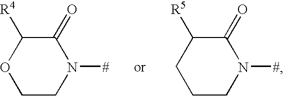

where
# is the point of attachment to the phenyl ring,
R⁴ represents hydrogen,
R⁵ represents hydrogen, hydroxyl or hydroxymethyl.

Preference is also given to compounds of the formula (I) in which R¹ represents a group of the formula

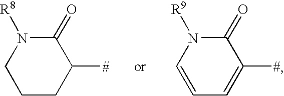

where
# is the point of attachment to the phenyl ring,
R⁸ represents hydrogen or methyl,
R⁹ represents hydrogen or methyl.

Preference is also given to compounds of the formula (I) in which R¹ represents a group of the formula

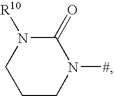

where
# is the point of attachment to the phenyl ring,
R¹⁰ represents methyl, ethyl or 2-hydroxyeth-1-yl.

Preference is also given to compounds of the formula (I) in which R² represents fluorine or chlorine.

Particular preference is given to compounds of the formula (I), in which R² represents fluorine.

Preference is also given to compounds of the formula (I), in which R³ represents hydrogen.

Preference is also given to compounds of the formula (I), in which R² represents fluorine and R³ represents hydrogen.

Particular preference is also given to the compound 5-chloro-N-({(5S)-3-[2-fluoro-4-(3-oxomorpholin-4-yl)phenyl]-2-oxo-1,3-oxazolidin-5-yl}methyl)thiophene-2-carboxamide of the formula

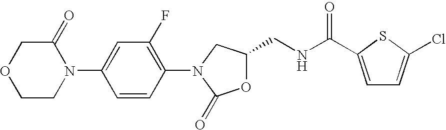

and its salts, its solvates and the solvates of its salts. The compound is described in Example 1.

Particular preference is also given to the compound 5-chloro-N-({(5S)-3-[2-fluoro-4-(2-oxopiperidin-1-yl)phenyl]-2-oxo-1,3-oxazolidin-5-yl}methyl)thiophene-2-carboxamide of the formula

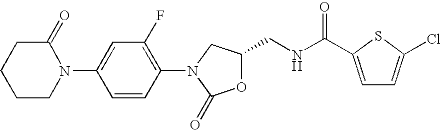

and its salts, its solvates and the solvates of its salts. The compound is described in Example 11.

Particular preference is also given to the compound 5-chloro-N-({(5S)-3-[2-chloro-4-(3-oxomorpholin-4-yl)phenyl]-2-oxo-1,3-oxazolidin-5-yl}methyl)thiophene-2-carboxamide of the formula

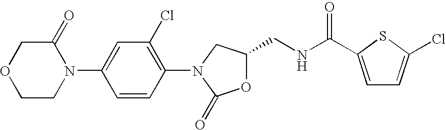

and its salts, its solvates and the solvates of its salts. The compound is described in Example 12.

Particular preference is also given to the compound 5-chloro-N-{[(5S)-3-{2-fluoro-4-[3-(hydroxymethyl)-2-oxopyridin-1(2H)-yl]phenyl}-2-oxo-1,3-oxazolidin-5-yl]methyl}thiophene-2-carboxamide of the formula

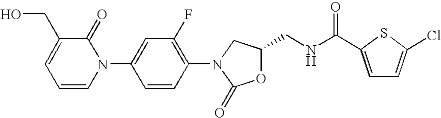

and its salts, its solvates and the solvates of its salts. The compound is described in Example 22.

The specific radical definitions given in the respective combinations or preferred combinations of radicals are, independently of the respective given combinations of radicals, also replaced by radical definitions of other combinations.

Very particular preference is given to combinations of two or more of the preferred ranges mentioned above.

The invention furthermore provides a process for preparing the compounds of the formula (I), or salts, solvates or solvates of the salts thereof, wherein [A] the compound of the formula

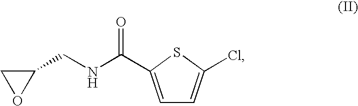

is, in the first step, reacted with compounds of the formula

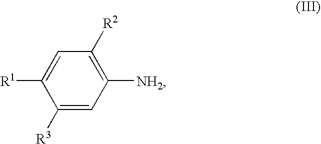

in which R¹, R² and R³ have the meaning given above,
to give compounds of the formula

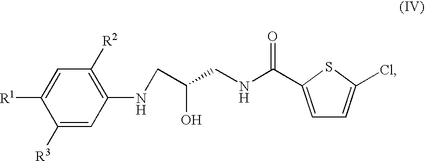

in which R¹, R² and R³ have the meaning given above,
and, in the second step, this compound is cyclised in the presence of phosgene or phosgene equivalents such as, for example, carbonyldiimidazole (CDI), to give the compounds of the formula (I)
or
[B] the compounds of the formula

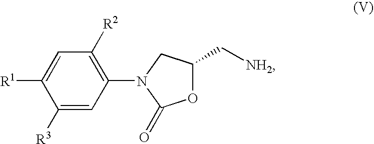

in which R¹, R² and R³ have the meaning given above,
are reacted with the compounds of the formula

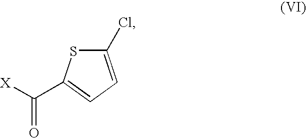

in which
X represents halogen, preferably bromine or chlorine, or hydroxyl.

If hydroxyl groups are protected during the process, for example by a silyl protective group, these are removed after the process [A] or [B] has ended using methods known to the person skilled in the art, for example by reaction with tetrabutylammonium fluoride in a solvent, such as, for example, tetrahydrofuran.

The free base of the salts can be obtained, for example, by chromatography on a reversed phase column using an acetonitrile/water gradient with addition of a base, in particular by using an RP18 Phenomenex Luna C18(2) column and diethylamine as base, or by dissolving the salts in an organic solvent and extracting with aqueous solutions of basic salts such as sodium bicarbonate.

The invention furthermore provides a process for preparing the compounds of the formula (I) or solvates thereof wherein salts of the compounds or solvates of the salts of the compounds are converted by chromatography with addition of a base into the compounds.

The reaction of the first step of process [A] is generally carried out in inert solvents, in the presence of a Lewis acid, preferably in a temperature range of from room temperature to reflux of the solvent at atmospheric pressure.

Inert solvents are, for example, polar aprotic solvents, such as, for example, acetonitrile, butyronitrile, dichloromethane or chloroform; preference is given to acetonitrile.

Lewis acids are, for example, magnesium perchlorate, ytterbium(III) trifluoromethanesulphonate, or aluminium trichloride; preference is given to magnesium perchlorate.

The reaction of the second step of process [A] is generally carried out in inert solvents, in the presence of a base, preferably in a temperature range of from room temperature to reflux of the solvent at atmospheric pressure.

Inert solvents are, for example, polar aprotic solvents, such as, for example, acetonitrile or butyronitrile.

Bases are, for example, strong tertiary amine bases, such as, for example, 4-N,N-dimethylaminopyridine.

Preference is given to the reaction with N,N′-carbonyldiimidazole as carbonic acid equivalent with addition of 4-N,N-dimethylaminopyridine as base.

If, in process [B], X is halogen, the reaction is generally carried out in inert solvents, if appropriate in the presence of a base, preferably in a temperature range of from −30° C. to 50° C. at atmospheric pressure.

Inert solvents are, for example, tetrahydrofuran, methylene chloride, pyridine, dioxane or dimethylformamide, preference is given to tetrahydrofuran or methylene chloride.

Bases are, for example, triethylamine, diisopropylethylamine or N-methylmorpholine;

preference is given to diisopropylethylamine.

If, in process [B], X is hydroxyl, the reaction is generally carried out in inert solvents, in the presence of a dehydrating agent, if appropriate in the presence of a base, preferably in a temperature range of from −30° C. to 50° C. at atmospheric pressure.

Inert solvents are, for example, halogenated hydrocarbons, such as dichloromethane or trichloromethane, hydrocarbons, such as benzene, nitromethane, dioxane, dimethylformamide or acetonitrile. It is also possible to use mixtures of the solvents. Particular preference is given to dichloromethane or dimethylformamide.

Here, suitable dehydrating agents are, for example, carbodiimides, such as, for example, N,N′-diethyl-, N,N,′-dipropyl-, N,N′-diisopropyl-, N,N′-dicyclohexylcarbodiimide, N-(3-dimethylaminoisopropyl)-N′-ethylcarbodiimide hydrochloride (EDC), N-cyclohexylcarbodiimide-N′-propyloxymethyl-polystyrene (PS-carbodiimide) or carbonyl compounds, such as carbonyldiimideazole, or 1,2-oxazolium compounds, such as 2-ethyl-5-phenyl-1,2-oxazolium 3-sulphate or 2-tert-butyl-5-methylisoxazolium perchlorate, or acylamino compounds, such as 2-ethoxy-1-ethoxycarbonyl-1,2-dihydroquinoline, or propanephosphonic anhydride, or isobutyl chloroformate, or bis(2-oxo-3-oxazolidinyl)phosphoryl chloride or benzotriazolyloxy-tri(dimethylamino)phosphonium hexafluorophosphate, or O-(benzotriazol-1-yl)-N,N,N,′N′-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N,′N′-tetramethyluronium hexafluorophosphate (HATU), or 1-hydroxybenzotriazole (HOBt), or benzotriazol-1-yloxytris(dimethylamino)-phosphonium hexafluorophosphate (BOP), or N-hydroxysuccinimide, or mixtures of these, with bases.

Bases are, for example, alkali metal carbonates, such as, for example, sodium carbonate or potassium carbonate, or sodium bicarbonate or potassium bicarbonate, or organic bases, such as trialkylamines, for example triethylamine, N-methylmorpholine, N-methylpiperidine, 4-dimethylaminopyridine or diisopropylethylamine.

The condensation with HATU or with EDC is preferably carried out in the presence of HOBt.

The compounds of the formulae (II) and (VI) are known or can be synthesized by known processes from the corresponding starting materials.

The compounds of the formula (III) in which the group of the formula R¹ is attached via a nitrogen atom to the phenyl ring are known or can be prepared by reacting compounds of the formula

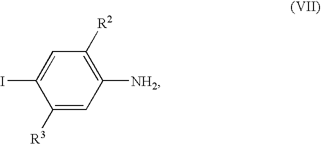

in which R² and R³ have the meaning given above
with compounds of the formula

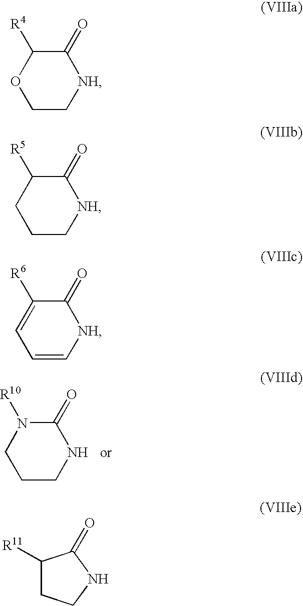

in which R⁴, R⁵, R⁶, R¹⁰ and R¹¹ have the meaning given above.

The reaction is generally carried out in inert solvents, in the presence of a copper(I) salt, a base and a diamine ligand, preferably in a temperature range of from 60° C. to reflux of the solvent at atmospheric pressure.

Inert solvents are, for example, aprotic solvents, such as toluene, dioxane, tetrahydrofuran or dimethylformamide; preference is given to dioxane.

Copper(I) salts are, for example, copper(I) iodide, copper(I) chloride or copper(I) oxide; preference is given to copper(I) iodide.

Bases are, for example, potassium phosphate, potassium carbonate or caesium carbonate; preference is given to potassium phosphate.

Diamine ligands are, for example, 1,2-diamines, such as N,N′-dimethylethylenediamine or 1,2-diaminocyclohexane; preference is given to N,N′-dimethylethylenediamine.

The compounds of the formulae (VII), (VIIIa), (VIIIb), (VIIIc), (VIIId) and (VIIIe) are known or can be synthesized by known processes from the corresponding starting materials.

In an alternative process, the compounds of the formula (VII) in the synthesis described above can be replaced by compounds of the formula

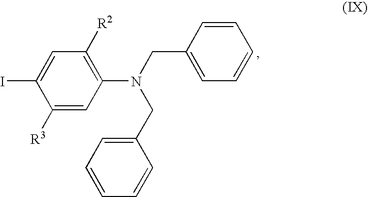

in which R² and R³ have the meaning given above.

The reaction is followed by hydrogenolytic cleavage of the benzyl groups using reaction conditions known to the person skilled in the art, to give the compounds of the formula (III). Reaction examples are given in the examples.

The compounds of the formula (IX) are known or can be synthesized by known processes from the corresponding starting materials.

The compounds of the formula (III) in which the group of the formula R¹ is saturated and attached via a carbon atom to the phenyl ring are known or can be prepared by reacting, in the first step, compounds of the formula

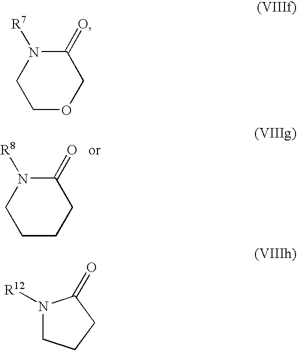

in which R⁷, R⁸ and R¹² have the meaning given above
with a strong base and a zinc salt
and, in the second step, without prior isolation, reacting the intermediate with compounds of the formula (IX) and a palladium complex,
and, in the third step, removing the benzyl groups hydrogenolytically using reaction conditions known to the person skilled in the art.

The reaction of the first step is generally carried out in inert solvents, preferably in a temperature range of from −30° C. to 0° C. at atmospheric pressure.

The reaction of the second step is generally carried out in inert solvents, preferably in a temperature range of from room temperature to reflux of the solvent at atmospheric pressure.

Inert solvents for both reaction steps are, for example, ethers, such as tetrahydrofuran, dioxane or 1,2-dimethoxyethane, if appropriate in a mixture with hydrocarbons, such as, for example, hexane; preference is given to tetrahydrofuran.

Strong bases are, for example, sec-butyllithium, tert-butyllithium, lithium diisopropylamide or lithium hexamethyldisilazide; preference is given to sec-butyllithium.

The zinc salt is, for example, zinc chloride.

Palladium complexes are formed in situ from palladium compounds and ligands. Suitable palladium compounds are, for example, palladium(II) acetate, palladium(II) chloride, bis(triphenylphosphine)palladium(II) chloride, tetrakis(triphenylphosphine)palladium(0), bis(di-benzylideneacetone)palladium(0); preference is given to bis(dibenzylideneacetone)palladium(0).

Ligands are, for example, 2-dicyclohexylphosphino-2′-(N,N-dimethylamino)biphenyl, binaphthyl or N-heterocyclic carbene ligands; preference is given to 2-dicyclohexylphosphino-2′-(N,N-dimethylamino)biphenyl.

The compounds of the formulae (VIIIf), (VIIIg) and (VIIIh) are known or can be synthesized by known processes from the corresponding starting materials.

The compounds of the formula (III) in which the group of the formula R¹ is unsaturated and attached via a carbon atom to the phenyl ring are known or can be prepared by, in the first step, reacting compounds of the formula

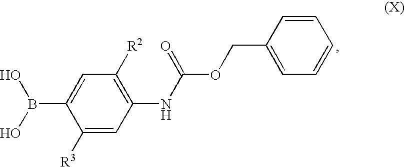

in which R² and R³ have the meaning given above
with compounds of the formula

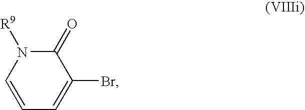

in which R⁹ has the meaning given above,
and, in the second step, removing the benzyloxycarbonyl protective group, to obtain the compounds of the formula (III).

The reaction of the first step is generally carried out in inert solvents, if appropriate in the presence of a little water, in the presence of a base and a palladium catalyst, and also, if appropriate, in the presence of a ligand, preferably in a temperature range of from 40° C. to reflux of the solvent at atmospheric pressure.

Inert solvents are, for example, ethers, such as tetrahydrofuran, dioxane or 1,2-dimethoxyethane; preference is given to 1,2-dimethoxyethane.

Bases are, for example, sodium carbonate, potassium carbonate or caesium carbonate; preference is given to a 2 molar solution of sodium carbonate in water.

Palladium compounds are, for example, palladium(II) acetate, palladium(II) chloride, bis(triphenylphosphine)palladium(II) chloride, tetrakis(triphenylphosphine)palladium(0); preference is given to tetrakis(triphenylphosphine)palladium(0).

Ligands are, for example, phosphine ligands which are stable to hydrolysis, such as triphenylphosphine.

The reaction of the second step is generally carried out in inert solvents, in the presence of an acid, preferably in a temperature range of from 0° C. to room temperature at atmospheric pressure.

Inert solvent/acid mixtures are, for example, hydrochloric acid in dioxane or trifluoroacetic acid in dichloromethane. Preference is given to hydrochloric acid in dioxane at room temperature.

The compounds of the formulae (X) and (VIIIi) are known or can be synthesized by known processes from the corresponding starting materials.

In an alternative process, the compounds of the formula (III) can be prepared by reducing the nitro group in compounds of the formula

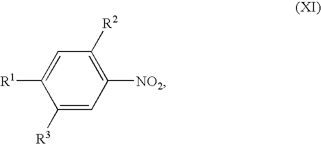

in which R¹, R² and R³ have the meaning given above.

The reaction is generally carried out using a reducing agent in inert solvents, preferably in a temperature range of from room temperature to reflux of the solvents at from atmospheric pressure to 3 bar.

Reducing agents are, for example, palladium on activated carbon and hydrogen, tin dichloride or titanium trichloride; preference is given to palladium on activated carbon and hydrogen or tin dichloride.

Inert solvents are, for example, ethers, such as diethyl ether, methyl tert-butyl ether, 1,2-dimethoxyethane, dioxane, tetrahydrofuran, glycol dimethyl ether or diethylene glycol dimethyl ether, alcohols, such as methanol, ethanol, n-propanol, isopropanol, n-butanol or tert-butanol, hydrocarbons, such as benzene, xylene, toluene, hexane, cyclohexane or mineral oil fractions, or other solvents, such as dimethylformamide, dimethylacetamide, acetonitrile or pyridine; preferred solvents are methanol, ethanol, isopropanol or, in the case of tin dichloride, dimethylformamide.

The compounds of the formula (XI) are known or can be synthesized by known processes from the corresponding starting materials.

The compounds of the formula (V) are known or can be prepared by removing the phthalimide protective group in compounds of the formula

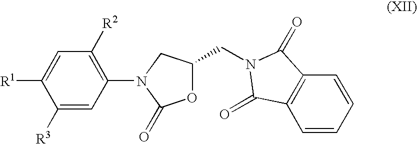

in which R¹, R² and R³ have the meaning given above.

The reaction is generally carried out using an aqueous methylamine solution or a hydrazine hydrate solution in ethanol, preferably using an aqueous methylamine solution at reflux of the solvents under atmospheric pressure.

The compounds of the formula (XII) are known, can be prepared from the corresponding epoxides as described under process [A] or can be synthesized by known processes from the corresponding starting materials.

The preparation of the compounds according to the invention can be illustrated by the synthesis schemes below:

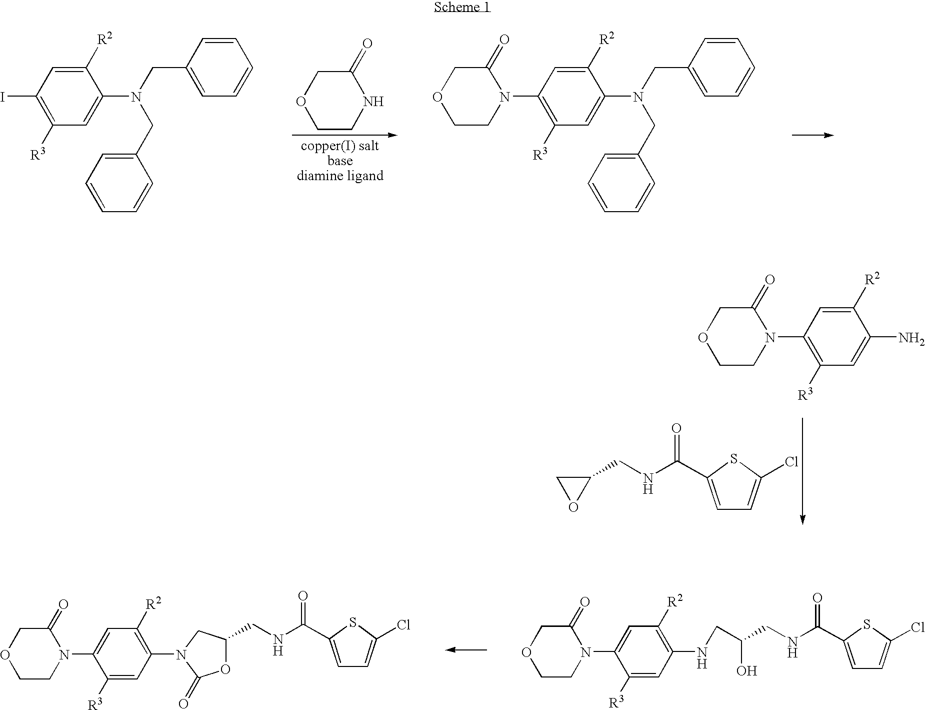

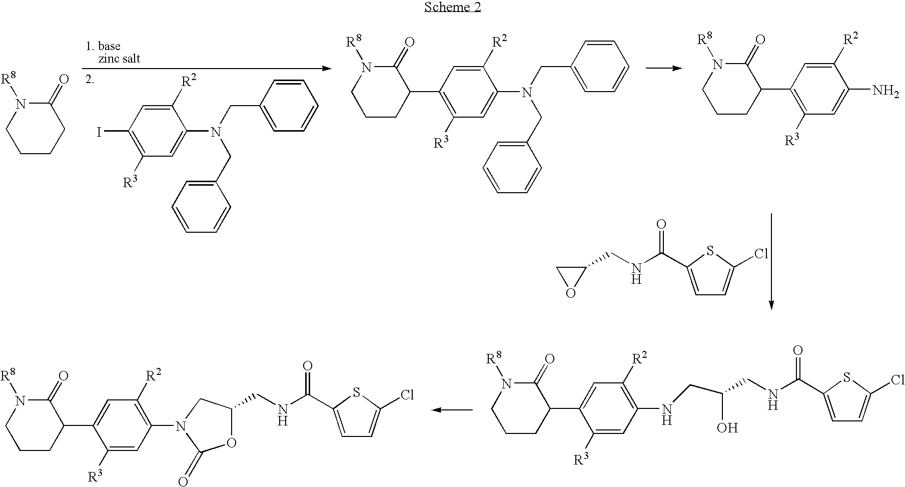

The compounds according to the invention have an unforeseeable useful spectrum of pharmacological activity.

Accordingly they are suitable for use as medicaments for the treatment and/or prophylaxis of diseases in humans and animals.

The compounds according to the invention are inhibitors of blood coagulation factor Xa acting, in particular, as anticoagulants.

In addition, the compounds according to the invention have favourable physicochemical properties and a large therapeutic width, which is advantageous for their therapeutic application.

The present invention furthermore provides the use of the compounds according to the invention for the treatment and/or prophylaxis of disorders, preferably thromboembolic disorders and/or thromboembolic complications.

“Thromboembolic disorders” in the sense of the present invention are in particular disorders such as myocardial infarction with ST segment elevation (STEMI) and without ST segment elevation (non-STEMI), stable angina pectoris, unstable angina pectoris, reocclusions and restenoses after coronary interventions such as angioplasty or aortocoronary bypass, peripheral arterial occlusion diseases, pulmonary embolisms, deep venous thromboses and kidney venous thromboses, transitory ischaemic attacks and also thrombotic and thromboembolic stroke.

Accordingly, the substances according to the invention are also suitable for the prevention and treatment of cardiogenic thromboembolisms, such as, for example, cerebral ischaemias, stroke and systemic thromboembolisms and ischaemias, in patients having acute, intermittent or persistent cardial arrhythmias, such as, for example, atrial fibrillation, and those undergoing cardioversion, furthermore in patients having cardiac valve disorders or having artifical cardiac valves.

Thromboembolic complications are furthermore encountered in microangiopathic haemolytic anaemias, extracorporeal circulatory systems, such as haemodialysis and prosthetic heart valves.

Moreover, the compounds according to the invention are also suitable for the prophylaxis and/or treatment of atherosclerotic vascular disorders and inflammatory disorders such as rheumatic disorders of the locomotor apparatus, and in addition also for the prophylaxis and/or treatment of Alzheimer's disease. Moreover, the compounds according to the invention can be used for inhibiting tumour growth and formation of metastases, for microangiopathies, age-related macula degeneration, diabetic retinopathy, diabetic nephropathy and other microvascular disorders, and also for the prevention and treatment of thromboembolic complications, such as, for example, venous thromboembolisms, for tumour patients, in particular those undergoing major surgical interventions or chemo- or radiotherapy.

Moreover, the compounds according to the invention are also suitable for the prophylaxis and/or treatment of pulmonary hypertension.

The term “pulmonary hypertension” includes certain forms of pulmonary hypertension. Examples which may be mentioned are pulmonary arterial hypertension, pulmonary hypertension associated with disorders of the left heart, pulmonary hypertension associated with pulmonary disorders and/or hypoxia and pulmonary hypertension owing to chronic thromboembolisms (CTEPH).

The term “pulmonary arterial hypertension” includes certain forms of pulmonary hypertension, as determined, for example, by the World Health Organization (WHO) (Clinical Classification of Pulmonary Hypertension, Venice 2003).

Pulmonary arterial hypersion comprises idiopathic pulmonary arterial hypertension (IPAH, formally also referred to as primary pulmonary hypertension), familiar pulmonary arterial hypertension (FPAH) and associated pulmonary-arterial hypertension (APAH), which is associated with collagenoses, congenital systemic-pulmonary shunt vitia, portal hypertension, HIV infections, the ingestion of certain drugs and medicaments, with other disorders (thyroid disorders, glycogen storage disorders, Morbus Gaucher, hereditary teleangiectasy, haemoglobinopathies, myeloproliferative disorders, splenectomy), with disorders having a significant venous/capillary contribution, such as pulmonary-venoocclusive disorder and pulmonary-capillary haemangiomatosis, and also persisting pulmonary hypertension of neonatants.

Pulmonary hypertension associated with disorders of the left heart comprises a diseased left atrium or ventricle and mitral or aorta valve defects.

Pulmonary hypertension associated with pulmonary disorders and/or hypoxia comprises chronic obstructive pulmonary disorders, interstitial pulmonary disorder, sleep apnoea syndrome, alveolar hypoventilation, chronic high-altitude sickness and inherent defects.

Pulmonary hypertension owing to chronic thromboembolisms (CTEPH) comprises the thromboembolic occlusion of proximal pulmonary arteries, the thromboembolic occlusion of distal pulmonary arteries and non-thrombotic pulmonary embolisms (tumour, parasites, foreign bodies).

The present invention furthermore provides the use of factor Xa inhibitors for preparing medicaments for the treatment and/or prophylaxis of pulmonary hypertension associated with sarcoidosis, histiocytosis X and lymphangiomatosis.

Moreover, the substances according to the invention may also be suitable for treating pulmonary and hepatic fibroses

Moreover, the compounds according to the invention may also be suitable for the treatment and/or prophylaxis of sepsis (or septicaemia), systemic inflammatory syndrome (SIRS), septic organ dysfunction, septic organ failure and multiorgan failure, acute respiratory distress syndrome (ARDS), acute lung injury (ALI), septic shock, DIC (disseminated intravascular coagulation or consumption coagulopathy) and/or septic organ failure.

“Sepsis” is defined as the presence of an infection and a systemic inflammatory response syndrome (hereinbelow referred to as “SIRS”). SIRS occurs associated with infections, but also other states such as injuries, burns, shock, operations, ischaemia, pancreatitis, reanimation or tumours. The definition of the ACCP/SCCM Consensus Conference Committee from 1992 (Crit. Care Med 1992; 20:864-874) describes the diagnosis symptoms and measuring parameters required for the diagnosis “SIRS” (inter alia body temperature change, increased pulse, breathing difficulties and changed blood picture). The later (2001) SCCM/ESICM/ACCP/ATS/SIS International Sepsis Definitions Conference essentially kept the criteria, but fine-tuned details (Levy et al., Crit. Care Med 2003; 31:1250-1256).

In the course of sepsis, there may be a generalized activation of the coagulation system (disseminated intravascular coagulation or consumption coagulopathy, hereinbelow referred to as “DIC”) with microthromboses in various organs and secondary haemorrhagic complications. Moreover, there may be endothelial damage with increased permeability of the vessels and seeping of fluids and proteins into the extravasal lumen. As the sepsis progresses, there may be failure of an organ (for example kidney failure, liver failure, respiratory failure, central-nervous deficits and/or cardiovascular failure) or multiorgan failure. “Septic shock” refers to the onset of hypotension requiring treatment, which hypotension promotes further organ damage and is associated with a worsening of the prognosis.

Pathogens may be bacteria (Gram-negative and Gram-positive), fungi, viruses and/or eukaryotes. Entrance point or primary infection may be, for example, pneumonia, an infection of the urinary tract or peritonitis. Infection can be, but is not necessarily, associated with bacteraemia.

DIC and/or SIRS may occur during sepsis, but also as a result of operations, tumour diseases, burns or other injuries. In DIC, there is a massive activation of the coagulatory system at the surface of damaged endothelial cells, the surfaces of foreign bodies or injured extravascular tissue. As a result, there is coagulation in small vessels of various organs with associated hypoxia and subsequent organ dysfunction. Secondary, there is a consumption of coagulation factors (for example factor X, prothrombin and fibrinogen) and platelets, which reduces the ability of the blood to coagulate and may result in serious bleeding.

Therapy of sepsis consists, firstly, of consequent elimination of the infectious cause, for example by operative focal reconstruction and antibiosis. Secondly, it consists in temporary intensive medical support of the affected organ systems. Therapies of various stages of this disease have been described, for example, in the following publication (Dellinger et al., Crit. Care Med 2004; 32:858-873). For DIC, there are no proven effective therapies.

The invention furthermore provides medicaments comprising a compound according to the invention and one or more further active compounds, in particular for the treatment and/or prophylaxis of the disorders mentioned above. Exemplary and preferred active compounds for combinations are:

### Antibiotic Therapy

Various antibiotics or antifungal medicament combinations are suitable, either as calculated therapy (prior to the presence of the microbrial diagnosis) or as specific therapy.

### Fluid Therapy

for example crystalloids or colloidal fluids.

### Vasopressors

for example norepinephrins, dopamines or vasopressin

### Inotropic Therapy

for example dobutamine

### Corticosteroids

for example hydrocortisone, or fludrocortisone

### Recombinant Human Activated Protein C

### Xigris

### Blood Products

for example erythrocyte concentrates, platelet concentrates, erythropietin or fresh frozen plasma

### Artificial Ventilation in the Case of Sepsis-Induced Acute Lung Injury (ALI) or Acute Respiratory Distress Syndrome (ARDS)

for example permissive hypercapnia, reduced tidal volumes

### Sedation, Analgaesia and Neuromuscular Blockade

Sedation: for example diazepam, lorazepam, midazolam or propofol. Opioids: for example fentanyl, hydromorphone, morphine, meperidine or remifentanil. NSAIDs: for example ketorolac, ibuprofen or acetaminophen. Neuromuscular blockade: for example pancuronium

### Glucose Control

for example insulin, glucose

### Renal Replacement Methods

for example continuous veno-venous haemofiltration or intermittent haemodialysis. Low doses of dopamine for renal protection.

### Anticoagulants

for example for thrombosis prophylaxis or renal replacement methods, for example unfractionated heparins, low-molecular-weight heparins, heparinoids, hirudin, bivalirudin or argatroban.

### Bicarbonate Therapy

### Stress Ulcer Prophylaxis

for example H2-receptor inhibitors, antacids.

In addition, the compounds according to the invention can also be used for preventing coagulation ex vivo, for example for preserving blood and plasma products, for cleaning/pretreatment of catheters and other medicinal aids and instruments, for coating synthetic surfaces of medicinal aids and instruments used in vivo or ex vivo or for biological samples comprising factor Xa.

The present invention furthermore provides the use of the compounds according to the invention for the treatment and/or prophylaxis of disorders, in particular the disorders mentioned above.

The present invention furthermore provides the use of the compounds according to the invention for preparing a medicament for the treatment and/or prophylaxis of disorders, in particular the disorders mentioned above.

The present invention furthermore provides a method for the treatment and/or prophylaxis of disorders, in particular the disorders mentioned above, using an anticoagulatory effective amount of the compound according to the invention.

The present invention furthermore provides a method for preventing the coagulation of blood in vitro, in particular in banked blood or biological samples containing factor Xa, which method is characterized in that an anticoagulatory effective amount of the compound according to the invention is added.

The present invention furthermore provides combinations of

compounds of the formula (I) with
other pharmaceutically active compounds, in particular with platelet aggregation inhibitors, anticoagulants, fibrinolytics, lipid-lowering substances, coronary therapeutics and/or vasodilators.

“Combinations” in the sense of the invention are to be understood as including not only administration forms comprising all components (so-called fixed combinations) and combination packages comprising the components separated from one another, but also components administered simultaneously or at different points in time, when they are used for the prophylaxis and/or treatment of the same disease. It is also possible to combine two or more active compounds with one another, these thus being two- or multi-component combinations.

The individual active compounds for combination are known from the literature, and most of them are commercially available.

Platelet aggregation inhibitors are, for example, acetylsalicylic acid (such as, for example, aspirin), ticlopidin (ticlid), clopidogrel (plavix) and prasugrel,

or integrin antagonists, such as, for example, glycoprotein-IIb/IIIa antagonists, such as, for example, abciximab, eptifibatide, tirofiban, lamifiban, lefradafiban and fradafiban.

Anticoagulatory effective substances (anticoagulants) are, for example, heparin (UFH), low-molecular-weight heparins (NMH), such as, for example, tinzaparin, certoparin, parnaparin, nadroparin, ardeparin, enoxaparin, reviparin, dalteparin, danaparoid,

### AVE 5026 (Sanofi-Aventis, Company Presentation 2008, February 12),

### M118 (Momenta Pharmaceuticals Inc., Press Release 2008, February 14),

### ORG42675 (Organon International Inc., Company World Wide Website 2007, April),

and direct thrombin inhibitors (DTI).

Direct thrombin inhibitors are, for example:

Exanta (ximelagatran)

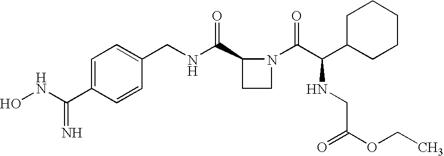

Rendix (dabigatran)

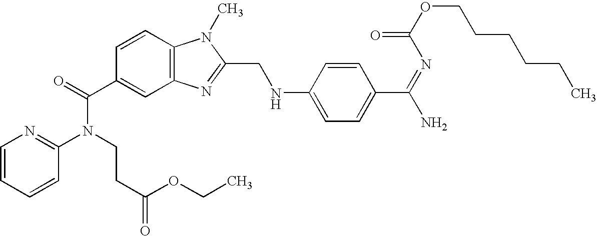

### AZD-0837 [AstraZeneca Annual Report 2006, 19 Mar. 2007]

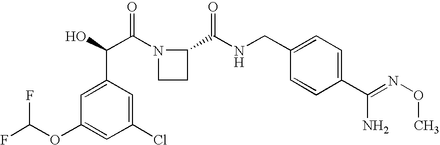

SSR-182289A [J. Lorrain et al. Journal of Pharmacology and Experimental Therapeutics 2003, 304, 567-574; J-M Altenburger et al. Bioorg. Med. Chem. 2004, 12, 1713-1730]

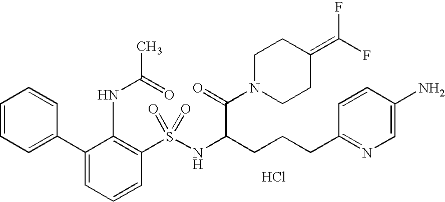

TGN-167 [S. Combe et al. Blood 2005, 106, abstract 1863 (ASH 2005)]
N—[(benzyloxy)carbonyl]-L-phenylalanyl-N-[(1S)-1-(dihydroxyboryl)-4-methoxybutyl]-D-prolinamide [WO 2005/084685]

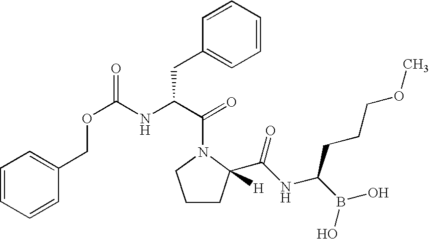

TGN-255 (flovagatran)

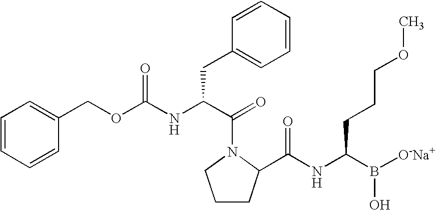

### Sofigatran [WHO Drug Information 2007, 21, 77]

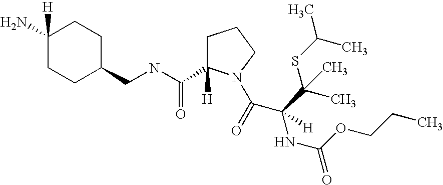

MCC-977 [Mitsubishi Pharma website pipeline 2006, 25. July 2006]

### MPC-0920 [Press Release: “Myriad Genetics Begins Phase 1 Trial of Anti-Thrombin Drug MPC-0920”, Myriad Genetics Inc, 02. May 2006]

Plasminogen activators (thrombolytics/fibrinolytics) are, for example, tissue plasminogen activator (t-PA), streptokinase, reteplase and urokinase.

Lipid-lowering substances are in particular HMG-CoA (3-hydroxy-3-methylglutaryl-coenzyme A) reductase inhibitors, such as, for example, lovastatin (mevacor; U.S. Pat. No. 4,231,938), simvastatin (zocor; U.S. Pat. No. 4,444,784), pravastatin (pravachol; U.S. Pat. No. 4,346,227), fluvastatin (lescol; U.S. Pat. No. 5,354,772) and atorvastatin (lipitor; U.S. Pat. No. 5,273,995).

Coronary therapeutic/vasodilators are in particular ACE (angiotensin converting enzyme) inhibitors, such as, for example, captopril, lisinopril, enalapril, ramipril, cilazapril, benazepril, fosinopril, quinapril and perindopril, or AII (angiotensin II) receptor antagonists, such as, for example, embusartan (U.S. Pat. No. 5,863,930), losartan, valsartan, irbesartan, candesartan, eprosartan and temisartan, or β-adrenoceptor antagonists, such as, for example, carvedilol, alprenolol, bisoprolol, acebutolol, atenolol, betaxolol, carteolol, metoprolol, nadolol, penbutolol, pindolol, propanolol and timolol, or alpha-1-adrenoceptor antagonists, such as, for example, prazosine, bunazosine, doxazosine and terazosine, or diuretics, such as, for example, hydrochlorothiazide, furosemide, bumetanide, piretanide, torasemide, amiloride and dihydralazine, or calcium channel blockers, such as, for example, verapamil and diltiazem, or dihydropyridine derivatives, such as, for example, nifedipin (Adalat) and nitrendipine (Bayotensin), or nitro preparations, such as, for example, isosorbide 5-mononitrate, isosorbide dinitrate and glycerol trinitrate, or substances causing an increase in cyclic guanosine monophosphate (cGMP), such as, for example, stimulators of soluble guanylate cyclase (WO 98/16223, WO 98/16507, WO 98/23619, WO 00/06567, WO 00/06568, WO 00/06569, WO 00/21954, WO 00/66582, WO 01/17998, WO 01/19776, WO 01/19355, WO 01/19780, WO 01/19778, WO 07/045,366, WO 07/045,367, WO 07/045,369, WO 07/045,370, WO 07/045,433).

The present invention further relates to medicaments which comprise at least one compound according to the invention, normally together with one or more inert, non-toxic, pharmaceutically suitable excipients, and to the use thereof for the aforementioned purposes.

The present invention furthermore provides medicaments comprising a compound according to the invention and one or more other of the active compounds for combination mentioned above, in particular for the treatment and/or prophylaxis of the disorders mentioned above.

The compounds according to the invention can act systemically and/or locally. For this purpose, they can be administered in a suitable way such as, for example, by the oral, parenteral, pulmonal, nasal, sublingual, lingual, buccal, rectal, dermal, transdermal, conjunctival, otic route or as implant or stent.

The compounds according to the invention can be administered in administration forms suitable for these administration routes.

Suitable for oral administration are administration forms which function according to the prior art and deliver the compounds according to the invention rapidly and/or in modified fashion, and which contain the compounds according to the invention in crystalline and/or amorphized and/or dissolved form, such as, for example, tablets (uncoated or coated tablets, for example having enteric coatings or coatings which are insoluble or dissolve with a delay and control the release of the compound according to the invention), tablets which disintegrate rapidly in the mouth, or films/wafers, films/lyophilisates, capsules (for example hard or soft gelatin capsules), sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous or intraperitoneal). Administration forms suitable for parenteral administration are, inter alia, preparations for injection and infusion in the form of solutions, suspensions, emulsions, lyophilisates or sterile powders.

Suitable for the other administration routes are, for example, pharmaceutical forms for inhalation (inter alia powder inhalers, nebulizers), nasal drops, solutions or sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (e.g. patches), milk, pastes, foams, dusting powders, implants or stents.

Oral or parenteral administration is preferred, especially oral administration.

The compounds according to the invention can be converted into the stated administration forms. This can take place in a manner known per se by mixing with inert, non-toxic, pharmaceutically suitable excipients. These excipients include, inter alia, carriers (for example microcrystalline cellulose, lactose, mannitol), solvents (e.g. liquid polyethylene glycols), emulsifiers and dispersants or wetting agents (for example sodium dodecyl sulphate, polyoxysorbitan oleate), binders (for example polyvinylpyrrolidone), synthetic and natural polymers (for example albumin), stabilizers (e.g. antioxidants such as, for example, ascorbic acid), colours (e.g. inorganic pigments such as, for example, iron oxides) and masking flavours and/or odours.

It has generally proved advantageous to administer on parenteral administration amounts of about 0.001 to 5 mg/kg, preferably about 0.01 to 1 mg/kg, of body weight to achieve effective results, and on oral administration the dosage is about 0.01 to 100 mg/kg, preferably about 0.01 to 20 mg/kg, and very particularly preferably 0.1 to 10 mg/kg, of body weight.

It may nevertheless be necessary where appropriate to deviate from the stated amounts, in particular as a function of the body weight, route of administration, individual response to the active ingredient, nature of the preparation and time or interval over which administration takes place. Thus, it may be sufficient in some cases to make do with less than the aforementioned minimum amount, whereas in other cases the stated upper limit must be exceeded. It may in the event of administration of larger amounts be advisable to divide these into a plurality of individual doses over the day.

The following exemplary embodiments illustrate the invention. The invention is not restricted to the examples.

The percentage data in the following tests and examples are, unless indicated otherwise, percentages by weight; parts are parts by weight. Solvent ratios, dilution ratios and concentration data for the liquid/liquid solutions are in each case based on volume.

### A. EXAMPLES

### Abbreviations

TLC Thin-layer chromatography
DCI Direct chemical ionization (in MS)
DMF N,N-Dimethylformamide
DMSO Dimethyl sulphoxide
d Day(s)
EDC N′-(3-dimethylaminopropyl)-N-ethylcarbodiimide×HCl
ee Enantiomeric excess
eq. Equivalent(s)
ESI Electrospray ionization (in MS)
h Hour(s)
HATU O-(7-azabenzotriazol-1-yl)-N,N,N′,N′-tetramethyluronium hexafluorophosphate
HPLC High-pressure, high-performance liquid chromatography
LC-MS Liquid chromatography-coupled mass spectroscopy
min Minute(s)
MS Mass spectroscopy
NMR Nuclear magnetic resonance spectroscopy
RP Reversed phase (in HPLC)
RT Room temperature
Rₜ Retention time (in HPLC)
THF Tetrahydrofuran
LC-MS and HPLC methods

Method 1: Instrument: HP 1100 with DAD detection; column: Kromasil 100 RP-18, 60 mm×2.1 mm, 3.5 μm; mobile phase A: 5 ml of perchloric acid (70% strength)/l of water, mobile phase B: acetonitrile; gradient: 0 min 2% B→0.5 min 2% B→4.5 min 90% B→6.5 min 90% B→6.7 min 2% B→7.5 min 2% B; flow rate: 0.75 ml/min; column temperature: 30° C.; UV detection: 210 nm.

Method 2: Instrument: HP 1100 with DAD detection; column: Kromasil 100 RP-18, 60 mm×2.1 mm, 3.5 μm; mobile phase A: 5 ml of perchloric acid (70% strength)/l of water, mobile phase B: acetonitrile; gradient: 0 min 2% B→0.5 min 2% B→4.5 min 90% B→9 min 0% B→9.2 min 2% B→10 min 2% B; flow rate: 0.75 ml/min; column temperature: 30° C.; UV detection: 210 nm.

Method 3: MS instrument type: Micromass ZQ; HPLC instrument type: Waters Alliance 2795; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

Method 4: MS instrument type: Micromass ZQ; HPLC instrument type: HP 1100 Series; UV DAD; column: Phenomenex Gemini 3μ 30 mm×3.00 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

Method 5: Instrument: Micromass Quattro LCZ with HPLC Agilent series 1100; column: Phenomenex Gemini 3μ 30 mm×3.00 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 208-400 nm.

Method 6: Instrument: Micromass Platform LCZ with HPLC Agilent series 1100; column: Thermo HyPURITY Aquastar 3μ 50 mm×2.1 mm; mobile phase A: 1 l of water+0.5 ml of 50% strength formic acid, mobile phase B: 1 l of acetonitrile+0.5 ml of 50% strength formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm.

Method 7: Instrument: HP 1100 with DAD detection; column: Kromasil 100 RP-18, 60 mm×2.1 mm, 3.5 μm; mobile phase A: 5 ml of perchloric acid (70% strength)/l of water, mobile phase B: acetonitrile; gradient: 0 min 2% B→0.5 min 2% B→4.5 min 90% B→15 min 90% B→15.2 min 2% B→16 min 2% B; flow rate: 0.75 ml/min; column temperature: 30° C.; UV detection: 210 nm.

Method 8: MS instrument type: Waters ZQ; HPLC instrument type: Waters Alliance 2795; column: Phenomenex Onyx Monolithic C18, 100 mm×3 mm; mobile phase A: 1 l of water+0.5 ml of 50% strength formic acid, mobile phase B: 1 l of acetonitrile+0.5 ml of 50% strength formic acid; gradient: 0.0 min 90% A→2 min 65% A→4.5 min 5% A→6 min 5% A; flow rate: 2 ml/min; oven: 40° C.; UV detection: 210 nm.

Method 9: Instrument: Micromass GCT, GC6890; column: Restek RTX-35MS, 30 m×250 μm×0.25 μm; constant helium flow: 0.88 ml/min; oven: 60° C.; inlet: 250° C.; gradient: 60° C. (maintained for 0.30 min), 50° C./min→120° C., 16° C./min→250° C., 30° C./min→300° C. (maintained for 1.7 min).

Method 10: Instrument: Micromass GCT, GC6890; column: Restek RTX-35, 15 m×200 μm×0.33 μm; constant helium flow: 0.88 ml/min; oven: 70° C.; inlet: 250° C.; gradient: 70° C., 30° C./min→310° C. (maintained for 3 min).

Method 11: Column: GROM-SIL 1200DS-4 HE, 10 μM, 250 mm×30 mm; flow rate: 50 ml/min; mobile phase and gradient program: acetonitrile/0.1% aqueous formic acid 10:90 (0-3 min), acetonitrile/0.1% aqueous formic acid 10:90→95:5 (3-27 min), acetonitrile/0.1% aqueous formic acid 95:5 (27-34 min), acetonitrile/0.1% aqueous formic acid 10:90 (34-38 min); temperature: 22° C.; UV detection: 254 nm.

### Starting Materials

### Example 1A

### 5-Chlorothiophene-2-carbonyl chloride

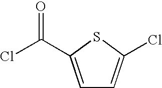

137 ml (1.57 mol) of oxalyl dichloride are added to a suspension of 51.2 g (0.315 mmol) of 5-chlorothiophene-2-carboxylic acid in 307 ml of dichloromethane. After addition of 2 drops of DMF, the mixture is stirred at room temperature for 15 hours. The solvent and excess oxalyl chloride are then removed on a rotary evaporator. The residue is distilled under reduced pressure. The product boils at 74-78° C. and a pressure of 4-5 mbar. This gives 50.5 g (87% of theory) of an oil which solidifies on storage in the fridge.

¹H-NMR (400 MHz, CDCl₃, δ/ppm): 7.79 (d, 1H), 7.03 (d, 1H).

GC/MS (method 9): Rₜ=5.18 min.

MS (EI+, m/z): 180/182/184 (2 ³⁵Cl/³⁷Cl) M⁺.

### Example 2A

### N—((S)-2,3-dihydroxypropyl)-5-chlorothiophene-2-carboxamide

(from: C. R. Thomas, Bayer HealthCare AG, DE-10300111-A1 (2004).)

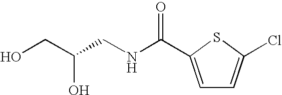

At 13-15° C., 461 g (4.35 mol) of sodium bicarbonate and 350 g (3.85 mol) of (2S)-3-aminopropane-1,2-diol hydrochloride are initially charged in 2.1 l of water, and 950 ml of 2-methyltetrahydrofuran are added. With cooling at 15-18° C., 535 g (2.95 mob) of 5-chlorothiophene-2-carbonyl chloride (compound from Example 1A) in 180 ml of toluene are added dropwise to this mixture over a period of two hours. For work-up, the phases are separated and a total of 1.5 l of toluene are added in a plurality of steps to the organic phase. The precipitated product is filtered off with suction, washed with ethyl acetate and dried. This gives 593.8 g (92% of theory) of product.

### Example 3A

### N—((S)-3-bromo-2-hydroxypropyl)-5-chlorothiophene-2-carboxamide

(from: C. R. Thomas, Bayer HealthCare AG, DE-10300111-A1 (2004).)

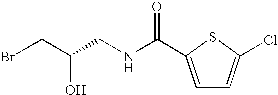

At 21-26° C., 301.7 ml of a 33% strength solution of hydrogen bromide in acetic acid are added over a period of 30 minutes to a suspension of 100 g (0.423 mol) of the compound from Example 2A in 250 ml of glacial acetic acid. 40 ml of acetic anhydride are then added, and the reaction mixture is stirred at 60-65° C. for three hours. At 20-25° C., 960 ml of methanol are then added over a period of 30 minutes. The reaction mixture is stirred under reflux for 2.5 hours and then at 20-25° C. overnight. For work-up, the solvents are distilled off under reduced pressure at about 95 mbar. 50 ml of n-butanol and 350 ml of water are added to the suspension that remains. The precipitated product is filtered off with suction, washed with water and dried. This gives 89.8 g (71% of theory) of product.

### Example 4A

### 5-Chloro-N-[(2S)-oxiran-2-ylmethyl]thiophene-2-carboxamide

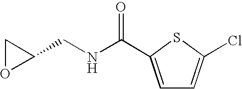

155 g (1.12 mol) of powdered potassium carbonate are added to a solution of 50 g (0.167 mol) of the compound from Example 3A in 500 ml of anhydrous THF, and the mixture is stirred at room temperature for 3 days. The inorganic salts are then filtered off with suction through a layer of kieselguhr, the filter cake is washed twice with in each case 100 ml of THF and the filtrate is concentrated at room temperature on a rotary evaporator. This gives 36 g (81% of theory) of product.

¹H-NMR (400 MHz, DMSO-d₆, δ/ppm): 8.81 (t, 1H), 7.68 (d, 1H), 7.19 (d, 1H), 3.55-3.48 (m, 1H), 3.29-3.22 (m, 1H), 3.10-3.06 (m, 1H), 2.75-2.72 (m, 1H), 2.57-2.54 (m, 1H).

HPLC (method 1): Rₜ=3.52 min.

MS (DCl, NH₃, m/z): (³⁵Cl/³⁷Cl) 218/220 (M+H)⁺, 235/237 (M+NH₄)⁺.

### Example 5A

### N,N-Dibenzyl-2-fluoro-4-iodoaniline

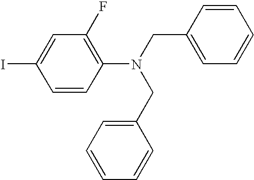

In a mixture of 100 ml of water and 200 ml of dichloromethane, 24.37 g (0.103 mol) of 2-fluoro-4-iodoaniline, 31.8 ml (0.267 mol) of benzyl bromide, 23.98 g (0.226 mol) of sodium carbonate and 1.9 g (5.14 mmol) of tetra-n-butylammonium iodide are heated at reflux for six days. After cooling to room temperature, the phases are separated. The organic phase is washed with water and saturated sodium chloride solution and dried over anhydrous sodium sulphate. After filtration, the solvent is removed on a rotary evaporator. The residue obtained is purified by filtration with suction through kieselguhr using cyclohexane as mobile phase. This gives 35 g (82% of theory) of the title compound.

¹H-NMR (400 MHz, DMSO-d₆, δ/ppm): 7.48 (1H, dd), 7.32-7.21 (m, 11H), 6.69 (dd, 1H), 4.33 (s, 4H).

HPLC (method 1): Rₜ=5.87 min.

MS (DCl, NH₃, m/z): 418 (M+H)⁺.

### Example 6A

### 4-[4-(Dibenzylamino)-3-fluorophenyl]morpholin-3-one

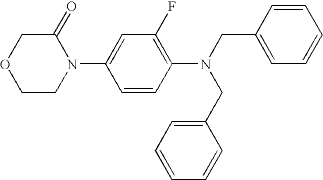

1.5 g (3.59 mmol) of the compound from Example 5A are dissolved in 20 ml of anhydrous dioxane, and 0.45 g (4.49 mmol) of morpholinone, 137 mg (0.719 mmol) of copper(I) iodide, 1.53 g (7.19 mmol) of potassium phosphate and 153 μl (1.44 mmol) of N,N′-dimethylethylenediamine are added in succession. By repeatedly applying a slight vacuum and venting with argon, the reflux apparatus is made inert. The reaction mixture is heated at reflux for 15 hours. After this period, the mixture is allowed to cool to room temperature. Water is added, and the mixture is extracted with ethyl acetate. The organic extract is washed successively with water and saturated sodium chloride solution. The extract is dried over anhydrous magnesium sulphate and then filtered, and the filtrate is freed from the solvent under reduced pressure. The residue is purified by filtration with suction through silica gel using cyclohexane/ethyl acetate 1:1 as mobile phase. This gives 1.38 g (98% of theory) of the title compound.

¹H-NMR (400 MHz, DMSO-d₆, δ/ppm): 7.32-7.28 (m, 9H), 7.26-7.20 (m, 2H), 7.00-6.92 (m, 2H), 4.33 (s, 4H), 4.15 (s, 2H), 3.91 (dd, 2H), 3.55 (dd, 2H).

HPLC (method 1): Rₜ=4.78 min.

MS (DCl, NH₃, m/z): 391 (M+H)⁺.

### Example 7A

### 4-(4-Amino-3-fluorophenyl)morpholin-3-one

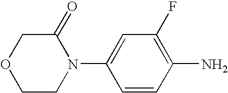

### Method 1:

700 mg (1.79 mmol) of the compound from Example 6A are dissolved in 70 ml of ethanol, and 95 mg of palladium on activated carbon (10%) are added. At room temperature and a hydrogen pressure of 1 bar, the mixture is hydrogenated for one hour. The catalyst is then filtered off through a little kieselguhr, and the filtrate is concentrated on a rotary evaporator. This gives 378 mg (95% of theory) of the title compound.

¹H-NMR (400 MHz, DMSO-d₆, δ/ppm): 7.04 (dd, 1H), 6.87 (dd, 1H), 6.73 (dd, 1H), 5.17 (s, broad, 2H), 4.12 (s, 2H), 3.91 (dd, 2H), 3.62 (dd, 2H).

HPLC (method 1): Rₜ=0.93 min.

MS (DCl, NH₃, m/z): 211 (M+H)⁺, 228 (M+NH₄)⁺.

### Method 2:

Under argon, a suspension of 29.6 g (125 mmol) of 2-fluoro-4-iodoaniline, 15.8 g (156 mmol, 1.25 eq.) of morpholin-3-one [J.-M. Lehn, F. Montavon, Helv. Chim. Acta 1976, 59, 1566-1583], 9.5 g (50 mmol, 0.4 eq.) of copper(I) iodide, 53.1 g (250 mmol, 2 eq.) of potassium phosphate and 8.0 ml (75 mmol, 0.6 eq.) of N,N′-dimethylethylenediamine in 300 ml of dioxane is stirred under reflux overnight. After cooling to RT, the reaction mixture is filtered through a layer of kieselguhr, and the residue is washed with dioxane. The combined filtrates are concentrated under reduced pressure. The crude product is purified by flash chromatography (silica gel 60, dichloromethane/methanol 100:1→100:3). This gives 24 g (74% of theory) of the title compound.

LC-MS (method 4): Rₜ=0.87 min;

MS (ESIpos): m/z=211 [M+H]⁺;

¹H-NMR (500 MHz, DMSO-d₆): δ=7.05 (dd, 1H), 6.87 (dd, 1H), 6.74 (dd, 1H), 5.14 (s, 2H), 4.11 (s, 2H), 3.92 (dd, 2H), 3.63 (dd, 2H).

### Example 8A

### 5-Chloro-N-[(2R)-3-{[2-fluoro-4-(3-oxomorpholin-4-yl)phenyl]amino}-2-hydroxypropyl]thiophene-2-carboxamide

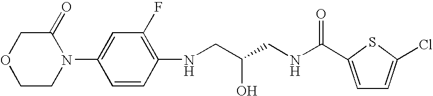

600 mg (2.69 mmol) of magnesium perchlorate are added to a solution of 376 mg (1.79 mmol) of the product from Example 7A and 429 mg (1.97 mmol) of the compound from Example 4A in 10 ml of acetonitrile, and the mixture is stirred at room temperature for 15 hours. Water is added, and the mixture is extracted with ethyl acetate. The organic extract is washed successively with water and saturated sodium chloride solution and dried over anhydrous magnesium sulphate. After filtration, the solvent is removed on a rotary evaporator. The residue is purified by preparative HPLC (method 11). This gives 503 mg (64% of theory) of the title compound.

¹H-NMR (400 MHz, DMSO-d₆, δ/ppm): 8.61 (t, 1H), 7.68 (d, 1H), 7.18 (d, 1H), 7.11 (dd, 1H), 6.97 (dd, 1H), 6.73 (dd, 1H), 5.33 (t, 1H), 5.14 (d, 1H), 4.13 (s, 2H), 3.92 (dd, 2H), 3.87-3.79 (m, 1H), 3.63 (dd, 2H), 3.39-3.22 (m, 2H, partially obscured by the signal for water), 3.21-3.15 (m, 1H), 3.08-3.02 (m, 1H).

HPLC (method 1): Rₜ=3.75 min.

MS (DCl, NH₃, m/z): 428/430 (³⁵Cl/³⁷Cl) (M+H)⁺, 445/447 (M+NH₄)⁺.

### Example 9A

### rac-1-(4-Amino-3-fluorophenyl)-3-hydroxypiperidin-2-one

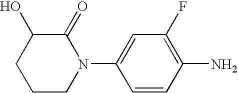

Analogously to the process described under Example 6A, 823 mg (3.47 mmol) of 2-fluoro-4-iodoaniline and 500 mg (4.34 mmol) of racemic 3-hydroxypiperidine (CAS No. 116908-80-6) give 703 mg (90% of theory) of the title compound.

¹H-NMR (400 MHz, DMSO-d₆, δ/ppm): 6.93 (dd, 1H), 6.77 (dd, 1H), 6.72 (dd, 1H), 5.11 (s, broad, 2H), 5.10 (d, 1H), 4.02-3.97 (m, 1H), 3.59-3.52 (m, 1H), 3.48-3.42 (m, 1H), 2.10-2.03 (m, 1H), 1.97-1.78 (m, 2H), 1.75-1.67 (m, 1H).

LC/MS (method 3): Rₜ=0.82 min.

MS (ES+, m/z): 225 (M+H)⁺.

### Example 10A

### rac-1-(4-Amino-3-fluorophenyl)-3-{[tert-butyl(dimethyl)silyl]oxy}piperidin-2-one

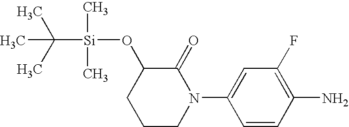

543 mg (3.60 mmol) of tert-butyldimethylsilyl chloride and 306 mg (4.50 mmol) of imidazole are added to a solution of 673 mg (3.00 mmol) of the compound from Example 9A in 10 ml of anhydrous DMF, and the mixture is stirred at room temperature. After two hours, most of the DMF is removed on a rotary evaporator, and the residue is taken up in dichloromethane and washed with water. Drying over anhydrous magnesium sulphate, filtration and concentration using a rotary evaporator give 963 mg (95% of theory) of the title compound.

¹H-NMR (400 MHz, DMSO-d₆, δ/ppm): 6.91 (dd, 1H), 6.77 (dd, 1H), 6.72 (dd, 1H), 5.10 (s, broad, 2H), 4.18 (dd, 1H), 3.59-3.52 (m, 1H), 3.47-3.41 (m, 1H), 2.08-2.02 (m, 1H), 1.97-1.91 (m, 1H), 1.87-1.80 (m, 2H), 0.87 (s, 9H), 0.08 (s, 3H), 0.05 (s, 3H).

LC/MS (method 4): Rₜ=2.71 min.

MS (ES+, m/z): 339 (M+H)⁺.

### Example 11A

### 5-Chloro-N-[(2R)-3-{[4-(3-{[dimethyl(1-methyl-1-silylethyl)silyl]oxy}-2-oxopiperidin-1-yl)-2-fluorophenyl]amino}-2-hydroxypropyl]thiophene-2-carboxamide (Mixture of diastereomers)

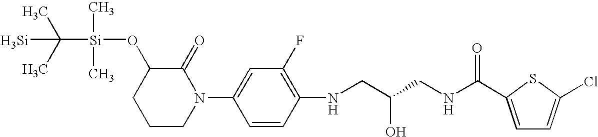

Analogously to the process described under Example 8A, 960 mg (2.84 mmol) of the product from Example 10A and 679 mg (3.12 mmol) of the compound from Example 4A give 902 mg (57% of theory) of the title compound.

¹H-NMR (400 MHz, DMSO-d₆, δ/ppm): 8.61 (t, 1H), 7.68 (d, 1H), 7.18 (d, 1H), 6.97 (dd, 1H), 6.85 (dd, 1H), 6.71 (dd, 1H), 5.27 (t, 1H), 5.13 (d, 1H), 4.19 (dd, 1H), 3.86-3.80 (m, 1H), 3.60-3.53 (m, 1H), 3.48-3.42 (m, 1H), 3.38-3.23 (m, 2H, partially obscured by the signal for water), 3.20-3.14 (m, 1H), 3.07-3.00 (m, 1H), 2.07-2.02 (m, 1H), 1.96-1.91 (m, 1H), 1.88-1.80 (m, 2H), 0.87 (s, 9H), 0.09 (s, 3H), 0.07 (s, 3H).

HPLC (method 1): Rₜ=5.18 min.

MS (ES+, m/z): 556/558 (³⁵Cl/³⁷Cl) (M+H)⁺.

### Example 12A

### N-({(5S)-3-[4-(3-{[tert-butyl(dimethyl)silyl]oxy}-2-oxopiperidin-1-yl)-2-fluorophenyl]-2-oxo-1,3-oxazolidin-5-yl}methyl)-5-chlorothiophene-2-carboxamide (Mixture of diastereomers)

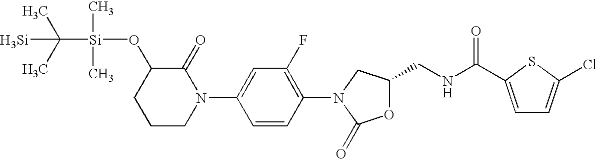

Analogously to the process described under Example 1, 879 mg (1.58 mmol) of the compound from Example 11A and 512 mg (3.16 mmol) of carbonyldiimidazole give 675 mg (73% of theory) of the title compound. The reaction time is 15 hours.

¹H-NMR (400 MHz, DMSO-d₆, δ/ppm): 8.99 (t, 1H), 7.70 (d, 1H), 7.49 (dd, 1H), 7.31 (dd, 1H), 7.20 (d, 1H), 7.17 (dd, 1H), 4.90-4.83 (m, 1H), 4.25 (dd, 1H), 4.11 (t, 1H), 3.80 (dd, 2H), 3.72-3.66 (m, 1H), 3.63-3.60 (m, 2H), 3.58-3.51 (m, 1H), 2.11-2.04 (m, 1H), 2.01-1.79 (m, 3H), 0.88 (s, 9H), 0.11 (s, 3H), 0.08 (s, 3H).

HPLC (method 3): Rₜ=2.84 min.

MS (DCl, NH₃, m/z): 599/601 (³⁵Cl/³⁷Cl) (M+NH₄)⁺.

### Example 13A

### rac-3-[4-(Dibenzylamino)-3-fluorophenyl]-1-methylpiperidin-2-one

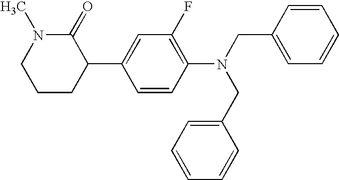

At 0° C., 5.14 ml (7.19 mmol) of a 1.4 molar solution of sec-butyllithium in cyclohexane are added slowly to a solution of 895 mg (7.91 mmol) of N-methylpiperidin-2-one in 16 ml of anhydrous THF. After the addition has ended, the mixture is stirred at 0° C. for 30 minutes. Slowly, 15.8 ml of a 0.5 molar solution of zinc dichloride in THF are then added. After a further 30 minutes at 0° C., this solution of the zinc enolate is, with the aid of a syringe, transferred into another flask containing a solution of 1.50 g (3.60 mmol) of, 103 mg (0.180 mmol) of bis(dibenzylideneacetone)palladium(0) and 106 mg (0.270 mmol) of 2-dicyclohexylphosphino-2′-(N,N-dimethylamino)biphenyl in 8 ml of anhydrous THF. The reaction mixture is heated at reflux for 19 hours. The THF is then removed on a rotary evaporator, and the residue is taken up in ethyl acetate and washed successively with water and saturated sodium chloride solution. After drying over anhydrous sodium sulphate, filtration and concentration using a rotary evaporator, the product is purified by flash chromatography on silica gel using cyclohexane/ethyl acetate 1:1 as mobile phase. This gives 1.12 g (78% of theory) of the title compound.

¹H-NMR (400 MHz, DMSO-d₆, δ/ppm): 7.32-7.28 (m, 8H), 7.24-7.19 (m, 2H), 7.93 (dd, 1H), 6.86 (dd, 1H), 6.73 (dd, 1H), 4.27 (s, 4H), 3.46 (dd, 1H), 3.40-3.33 (m, 1H), 3.31-3.24 (m, 1H, partially obscured by the signal for water), 2.83 (s, 3H), 2.01-1.94 (m, 1H), 1.85-1.69 (m, 3H).

HPLC (method 1): Rₜ=4.88 min.

MS (ES+, m/z): 403 (M+H)⁺.

### Example 14A

### rac-3-(4-Amino-3-fluorophenyl)-1-methylpiperidin-2-one

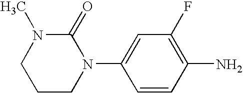

Analogously to the process described under Example 7A, 1.097 g (2.72 mmol) of the compound from Example 13A give 605 mg (99% of theory) of the title compound.

¹H-NMR (400 MHz, DMSO-d₆, δ/ppm): 6.77 (d, 1H), 6.67 (d, 1H), 6.65 (s, 1H), 4.93 (s, broad, 2H), 3.41-3.25 (m, 3H), 2.84 (s, 3H), 2.00-1.93 (m, 1H), 1.83-1.69 (m, 3H).

HPLC (method 1): Rₜ=2.68 min.

MS (ES+, m/z): 223 (M+H)⁺.

### Example 15A

### 5-Chloro-N-[(2R)-3-{[2-fluoro-4-(1-methyl-2-oxopiperidin-3-yl)phenyl]amino}-2-hydroxypropyl]-thiophene-2-carboxamide (Mixture of diastereomers)

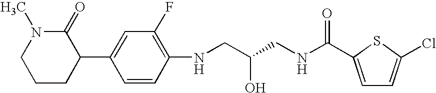

Analogously to the process described under Example 8A, 600 mg (2.70 mmol) of the product from Example 14A and 646 mg (2.97 mmol) of the compound from Example 4A give 758 mg (64% of theory) of the title compound.

¹H-NMR (400 MHz, DMSO-d₆, δ/ppm): 8.60 (t, 1H), 7.68 (d, 1H), 7.18 (d, 1H), 6.82 (dd, 1H), 6.75 (dd, 1H), 6.64 (dd, 1H), 5.12 (d, 1H), 5.07 (t, 1H), 3.85-3.78 (m, 1H), 3.43-3.34 (m, 3H), 3.33-3.23 (m, 2H, partially obscured by the signal for water), 3.18-3.12 (m, 1H), 3.03-2.98 (m, 1H), 2.85 (s, 3H), 2.01-1.94 (m, 1H), 1.86-1.69 (m, 3H).

HPLC (method 1): Rₜ=3.82 min.

MS (DCl, NH₃, m/z): 440/442 (³⁵Cl/³⁷Cl) (M+H)⁺, 457/459 (M+NH₄)⁺.

### Example 16A

### rac-3-({[tert-butyl(diphenyl)silyl]oxy}methyl)piperidin-2-one

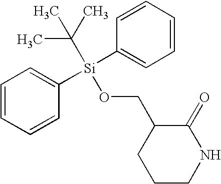

3.16 g (46.5 mmol) of imidazole and, dropwise, 11 ml (42.6 mmol) of tert-butyl(diphenyl)silyl chloride are added successively to a solution of 5.0 g (38.7 mmol) of racemic 3-hydroxymethylpiperidin-2-one (CAS No. 25219-43-6) in 40 ml of DMF. After three hours of stirring at room temperature, about 400 ml of water are added, and the mixture is extracted three times with ethyl acetate. The combined organic extracts are washed successively with saturated ammonium chloride solution, water and saturated sodium chloride solution. After drying over anhydrous magnesium sulphate, the mixture is filtered and the filtrate is freed from the solvent under reduced pressure. The residue obtained is purified by filtration with suction through silica gel using cyclohexane/ethyl acetate 20:1→1:1 as mobile phase. This gives 9.43 g (66% of theory) of the title compound.

¹H-NMR (400 MHz, CDCl₃, δ/ppm): 7.69-7.65 (m, 4H), 7.42-7.34 (m, 6H), 5.82 (s, broad, 1H), 4.03 (dd, 1H), 3.93 (dd, 1H), 3.32-3.28 (m, 2H), 2.53-2.48 (m, 1H), 2.07-1.99 (m, 1H), 1.96-1.87 (m, 2H), 1.78-1.68 (m, 1H), 1.04 (s, 9H).

HPLC (method 3): Rₜ=2.79 min.

MS (ESIpos, m/z): 368 (M+H)⁺.

### Example 17A

### rac-3-({[tert-Butyl(diphenyl)silyl]oxy}methyl)-1-[4-(dibenzylamino)-3-fluorophenyl]piperidin-2-one

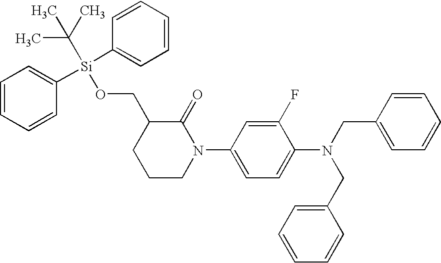

Analogously to the process described under Example 6A, 1.0 g (2.40 mmol) of the compound from Example 5A and 1.1 g (3.0 mmol) of the compound from Example 16A give 1.31 g (83% of theory) of the title compound.

¹H-NMR (400 MHz, DMSO-d₆, δ/ppm): 7.62-7.60 (m, 4H), 7.47-7.38 (m, 6H), 7.32-7.28 (m, 8H), 7.24-7.19 (m, 2H), 7.08 (dd, 1H), 6.92 (dd, 1H), 6.83 (dd, 1H), 4.31 (s, 4H), 4.03 (dd, 1H), 3.80 (dd, 1H), 3.57-3.53 (m, 2H), 2.60-2.54 (m, 1H), 2.07-1.92 (m, 3H), 1.88-1.81 (m, 1H), 1.00 (s, 9H).

LC/MS (method 2): Rₜ=6.88 min.

MS (ES+, m/z): 657 (M+H)⁺.

### Example 18A

### rac-1-(4-Amino-3-fluorophenyl)-3-({[tert-butyl(diphenyl)silyl]oxy}methyl)piperidin-2-one

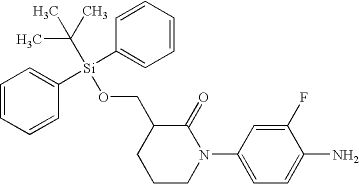

Analogously to the process described under Example 7A, 1.256 g (1.91 mmol) of the compound from Example 17A give 869 mg (95% of theory) of the title compound.

¹H-NMR (400 MHz, DMSO-d₆, δ/ppm): 7.65-7.63 (m, 4H), 7.48-7.40 (m, 6H), 6.87 (dd, 1H), 6.72 (dd, 1H), 6.70 (dd, 1H), 5.09 (s, broad, 2H), 4.05 (dd, 1H), 3.80 (dd, 1H), 3.56-3.51 (m, 2H), 2.58-2.52 (m, 1H), 2.09-1.93 (m, 3H), 1.90-1.80 (m, 1H), 1.00 (s, 9H).

HPLC (method 7): Rₜ=5.37 min.

MS (DCl, NH₃, m/z): 477 (M+H)⁺.

### Example 19A

### N-[(2R)-3-({4-[3-({[tert-Butyl(diphenyl)silyl]oxy}methyl)-2-oxopiperidin-1-yl]-2-fluorophenyl}-amino)-2-hydroxypropyl]-5-chlorothiophene-2-carboxamide (Mixture of diastereomers)

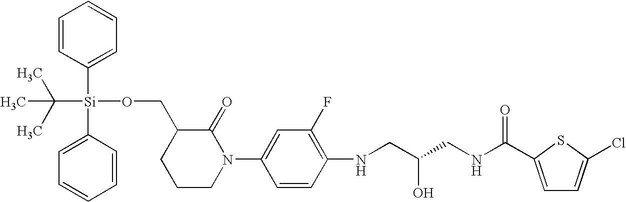

Analogously to the process described under Example 8A, 854 mg (1.79 mmol) of the product from Example 18A and 429 mg (1.97 mmol) of the compound from Example 4A give 785 mg (63% of theory) of the title compound. The reaction time is 2 days.

¹H-NMR (400 MHz, DMSO-d₆, δ/ppm): 8.62 (t, 1H), 7.68 (d, 1H), 7.66-7.62 (m, 4H), 7.48-7.40 (m, 6H), 7.17 (d, 1H), 6.93 (dd, 1H), 6.83 (dd, 1H), 6.70 (dd, 1H), 5.28 (t, 1H), 5.15 (d, 1H), 4.07 (dd, 1H), 3.84-3.78 (m, 2H), 3.57-3.53 (m, 2H), 3.38-3.23 (m, 2H, partially obscured by the signal for water), 3.20-3.14 (m, 1H), 3.07-3.00 (m, 1H), 2.59-2.53 (m, 1H), 2.09-1.94 (m, 3H), 1.90-1.82 (m, 1H), 1.00 (s, 9H).

HPLC (method 2): Rₜ=5.76 min.

MS (DCl, NH₃, m/z): 694/696 (³⁵Cl/³⁷Cl) (M+H)⁺.

### Example 20A

### N-{[(5S)-3-{4-[3-({[tert-Butyl(diphenyl)silyl]oxy}methyl)-2-oxopiperidin-1-yl]-2-fluorophenyl}-2-oxo-1,3-oxazolidin-5-yl]methyl}-5-chlorothiophene-2-carboxamide (Mixture of diastereomers)

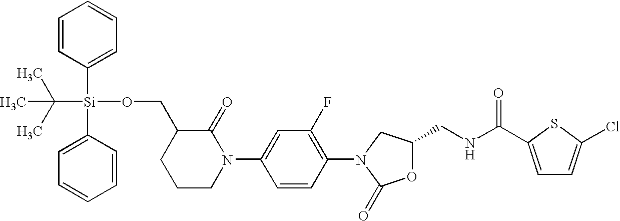

Analogously to the process described under Example 1, 763 mg (1.10 mmol) of the compound from Example 19A and 356 mg (2.20 mmol) of carbonyldiimidazole give 577 mg (73% of theory) of the title compound. The reaction time is 36 hours.

¹H-NMR (400 MHz, DMSO-d₆, δ/ppm): 8.97 (t, 1H), 7.70 (d, 1H), 7.64-7.62 (m, 4H), 7.50-7.40 (m, 7H), 7.27 (dd, 1H), 7.19 (d, 1H), 7.14 (dd, 1H), 4.90-4.83 (m, 1H), 4.13-4.05 (m, 2H), 3.82-3.78 (m, 2H), 3.67-3.59 (m, 4H), 2.67-2.61 (m, 1H), 2.11-1.99 (m, 3H), 1.96-1.86 (m, 1H), 1.00 (s, 9H).

HPLC (method 2): Rₜ=5.78 min.

MS (ES+, m/z): 720/722 (³⁵Cl/³⁷Cl) (M+H)⁺.

### Example 21A

### 1-[4-(Dibenzylamino)-3-fluorophenyl]piperidin-2-one

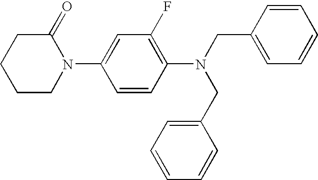

Analogously to the process described under Example 6A, 1.50 g (3.59 mmol) of the compound from Example 5A and 445 mg (4.49 mmol) of δ-valerolactam give 1.33 g (95% of theory) of the title compound. The reaction time is 24 hours.

¹H-NMR (400 MHz, DMSO-d₆, δ/ppm): 7.32-7.28 (m, 8H), 7.25-7.20 (m, 2H), 7.11 (dd, 1H), 6.92 (dd, 1H), 6.86 (dd, 1H), 4.30 (s, 4H), 3.52 (dd, 2H), 2.33 (dd, 2H), 1.83-1.75 (m, 4H).

LC/MS (method 1): Rₜ=4.92 min.

MS (ES+, m/z): 389 (M+H)⁺.

### Example 22A

### 1-(4-Amino-3-fluorophenyl)piperidin-2-one

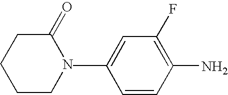

Analogously to the process described under Example 7A, 1.293 g (3.33 mmol) of the compound from Example 21A give 692 mg (99% of theory) of the title compound.

¹H-NMR (400 MHz, DMSO-d₆, δ/ppm): 6.91 (dd, 1H), 6.75 (dd, 1H), 6.70 (dd, 1H), 5.09 (s, broad, 2H), 3.49 (dd, 2H), 2.32 (dd, 2H), 1.84-1.76 (m, 4H).

HPLC (method 1): Rₜ=2.66 min.

MS (DCl, NH₃, m/z): 209 (M+H)⁺, 226 (M+NH₄)⁺.

### Example 23A

### 5-Chloro-N-[(2R)-3-{[2-fluoro-4-(2-oxopiperidin-1-yl)phenyl]amino}-2-hydroxypropyl]thiophene-2-carboxamide

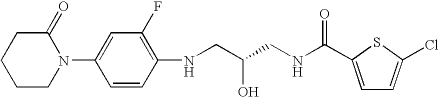

Analogously to the process described under Example 8A, 682 mg (3.27 mmol) of the product from Example 22A and 784 mg (3.60 mmol) of the compound from Example 4A give 1.22 g (87% of theory) of the title compound.

¹H-NMR (400 MHz, DMSO-d₆, δ/ppm): 8.60 (t, 1H), 7.67 (d, 1H), 7.17 (d, 1H), 6.98 (dd, 1H), 6.85 (dd, 1H), 6.70 (dd, 1H), 5.23 (t, 1H), 5.14 (d, 1H), 3.86-3.79 (m, 1H), 3.50 (dd, 2H), 3.38-3.23 (m, 2H, partially obscured by the signal for water), 3.20-3.14 (m, 1H), 3.07-3.00 (m, 1H), 2.32 (dd, 2H), 1.84-1.76 (m, 4H).

HPLC (method 1): Rₜ=3.90 min.

MS (ES+, m/z): 426/428 (³⁵Cl/³⁷Cl) (M+H)⁺.

### Example 24A

### 4-(4-Amino-3-chlorophenyl)morpholin-3-one

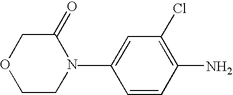

Analogously to the process described under Example 6A, 500 mg (1.97 mmol) of 2-chloro-4-iodoaniline and 249 mg (2.46 mmol) of morpholinone give 410 mg (92% of theory) of the title compound.

¹H-NMR (400 MHz, DMSO-d₆, δ/ppm): 7.22 (d, 1H), 7.00 (dd, 1H), 6.78 (d, 1H), 5.41 (s, broad, 2H), 4.13 (s, 2H), 3.91 (dd, 2H), 3.61 (dd, 2H).

HPLC (method 1): Rₜ=2.48 min.

MS (DCl, NH₃, m/z): 227/229 (³⁵Cl/³⁷Cl) (M+H)⁺, 244/246 (M+NH₄)⁺.

### Example 25A

### 5-Chloro-N-[(2R)-3-{[2-chloro-4-(3-oxomorpholin-4-yl)phenyl]amino}-2-hydroxypropyl]thiophene-2-carboxamide

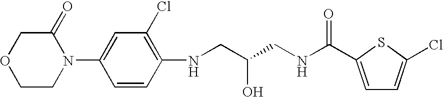

Analogously to the process described under Example 8A, 400 mg (1.77 mmol) of the product from Example 24A and 422 mg (1.94 mmol) of the compound from Example 4A give 424 mg (54% of theory) of the title compound. The reaction time is 40 hours.

¹H-NMR (500 MHz, DMSO-d₆, δ/ppm): 8.67 (t, 1H), 7.68 (d, 1H), 7.31 (d, 1H), 7.19 (d, 1H), 7.12 (dd, 1H), 6.76 (d, 1H), 5.35 (t, 1H), 5.27 (d, 1H), 4.13 (s, 2H), 3.92 (dd, 2H), 3.86-3.80 (m, 1H), 3.63 (dd, 2H), 3.36-3.27 (m, 2H, partially obscured by the signal for water), 3.26-3.20 (m, 1H), 3.11-3.06 (m, 1H).

HPLC (method 1): Rₜ=3.84 min.

MS (ES+, m/z): 444/446/448 (Cl₂, ³⁵Cl/³⁷Cl) (M+H)⁺.

### Example 26A

### 2-Fluoro-4-iodo-5-methylaniline

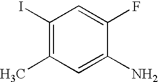

At a temperature of 0° C., a suspension of 1.0 g (7.99 mmol) of 2-fluoro-3-methylaniline and 1.34 g (15.98 mmol) of sodium bicarbonate in a mixture of in each case 5 ml of dichloromethane and methanol is, three times, evacuated until the solvent begins to boil and vented with argon. Dropwise (over a period of about 5 minutes), a solution of 3.12 g (7.99 mmol) of benzyltriethylammonium dichloroiodate(+I) (J. M. Tour et al., Org. Lett. 3(7), 991-992 (2001).) in 5 ml of dichloromethane is then added. The reaction mixture is subsequently stirred at room temperature for 30 minutes. Moderate evolution of gas. 20 ml of water are then added, and the organic phase is separated off, dried over anhydrous sodium sulphate, filtered and concentrated using a rotary evaporator. The crude product is purified by filtration with suction through silica gel using cyclohexane/ethyl acetate 4:1 as mobile phase. This gives 1.75 g (84% of theory) of a liquid.

¹H-NMR (400 MHz, DMSO-d₆, δ/ppm): 7.34 (d, 1H), 6.73 (d, 1H), 5.23 (s, broad, 2H), 2.19 (s, 3H).

LC/MS (method 4): Rₜ=2.27 min.

MS (ES+, m/z): 252 (M+H)⁺.

### Example 27A

### 4-(4-Amino-5-fluoro-2-methylphenyl)morpholin-3-one

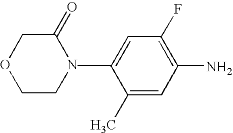

Analogously to the process described under Example 6A, 250 mg (0.996 mmol) of the compound from Example 26A and 242 mg (2.39 mmol) of morpholinone give 163 mg (68% of theory) of the title compound.

¹H-NMR (400 MHz, DMSO-d₆, δ/ppm): 6.91 (d, 1H), 6.63 (d, 1H), 5.12 (s, broad, 2H), 4.15 (2H), 3.92 (2H), broad signal for water, 1.97 (s, 3H).

HPLC (method 1): Rₜ=1.40 min.

MS (DCl, NH₃, m/z): 225 (M+H)⁺, 242 (M+NH₄)⁺.

### Example 28A

### 5-Chloro-N-[(2R)-3-{[2-fluoro-5-methyl-4-(3-oxomorpholin-4-yl)phenyl]amino}-2-hydroxypropyl]thiophene-2-carboxamide

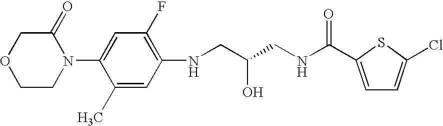

Analogously to the process described under Example 8A, 161 mg (0.718 mmol) of the product from Example 27A and 259 mg (1.15 mmol) of the compound from Example 4A give 212 mg (65% of theory) of the title compound and, at the same time, 27 mg (17% of theory) of the compound from Example 27A are recovered. The reaction time is 40 hours.

¹H-NMR (400 MHz, DMSO-d₆, δ/ppm): 8.63 (t, 1H), 7.69 (d, 1H), 7.19 (d, 1H), 6.97 (d, 1H), 6.60 (d, 1H), 5.32 (t, 1H), 5.15 (d, 1H), 4.16-4.16 (m, 2H), 3.94-3.92 (m, 2H), 3.86-3.79 (m, 1H), 3.63-3.56 (m, 1H), 3.43-3.25 (m, 2H, partially obscured by the signal for water), 3.26-3.16 (m, 2H), 3.07-3.01 (m, 1H), 1.98 (s, 3H).

HPLC (method 1): Rₜ=3.73 min.

MS (ES+, m/z): 442/444 (³⁵Cl/³⁷Cl) (M+H)⁺.

### Example 29A

### 1-(4-Amino-5-fluoro-2-methylphenyl)piperidin-2-one

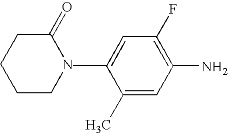

Analogously to the process described under Example 6A, 250 mg (0.996 mmol) of the compound from Example 26A and 237 mg (2.39 mmol) of δ-valerolactam give 195 mg (63% of theory) of the title compound, which, in spite of purification by preparative HPLC, is contaminated by valerolactam and has a content of only 72 mol %.

¹H-NMR (400 MHz, DMSO-d₆, δ/ppm): 6.80 (d, 1H), 6.59 (d, 1H), 5.03 (s, broad, 2H), 3.49-3.43 (m, 1H), 3.28-3.23 (m, 1H), 2.37-2.27 (m, 2H), 1.91 (s, 3H), 1.87-1.75 (m, 4H).

HPLC (method 1): Rₜ=2.74 min.

MS (DCl, NH₃, m/z): 223 (M+H)⁺, 240 (M+NH₄)⁺.

### Example 30A

### 5-Chloro-N-[(2R)-3-{[2-fluoro-5-methyl-4-(2-oxopiperidin-1-yl)phenyl]amino}-2-hydroxypropyl]-thiophene-2-carboxamide

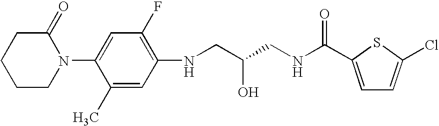

Analogously to the process described under Example 8A, 192 mg (0.622 mmol, purity 72%) of the product from Example 29A and 207 mg (0.95 mmol) of the compound from Example 4A give 191 mg (70% of theory) of the title compound.

¹H-NMR (400 MHz, DMSO-d₆, δ/ppm): 8.64 (t, 1H), 7.68 (d, 1H), 7.18 (d, 1H), 6.86 (d, 1H), 6.57 (d, 1H), 5.22-5.19 (m, 2H), 3.86-3.80 (m, 1H), broad signal for water, 3.08-2.99 (m, 1H), 2.37-2.28 (m, 2H), 1.93 (s, 3H), 1.87-1.77 (m, 4H).

HPLC (method 1): Rₜ=3.89 min.

MS (DCl, NH₃, m/z): 440/442 (³⁵Cl/³⁷Cl) (M+H)⁺, 457/459 (M+NH₄)⁺.

### Example 31A

### 1-[4-(Dibenzylamino)-3-fluorophenyl]-3-methyltetrahydropyrimidin-2(1H)-one

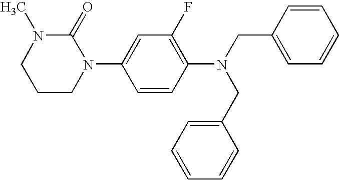

Analogously to the process described under Example 6A, 1.5 g (3.59 mmol) of the compound from Example 5A and 0.77 g (6.74 mmol) of 1-methyltetrahydropyrimidin-2(1H)-one (CAS No. 10166-54-8) are converted into 1.28 g (88% of theory) of the title compound. The reaction time is 40 hours.

¹H-NMR (400 MHz, DMSO-d₆, δ/ppm): 7.32-7.27 (m, 8H), 7.23-7.18 (m, 2H), 7.08 (dd, 1H), 6.86 (dd, 1H), 6.80 (dd, 1H), 4.24 (s, 4H), 3.54 (dd, 2H), 3.28 (dd, 2H), 2.81 (s, 3H), 1.99-1.93 (m, 2H).

HPLC (method 1): Rₜ=4.72 min.

MS (ES+, m/z): 404 (M+H)⁺.

### Example 32A

### 1-(4-Amino-3-fluorophenyl)-3-methyltetrahydropyrimidin-2(1H)-one

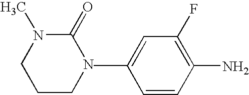

Analogously to the process described under Example 7A, 1.22 g (3.03 mmol) of the compound from Example 31A give 657 mg (97% of theory) of the title compound.

¹H-NMR (400 MHz, DMSO-d₆, δ/ppm): 6.88 (dd, 1H), 6.73 (dd, 1H), 6.66 (dd, 1H), 4.97 (s, broad, 2H), 3.51 (dd, 2H), 3.29 (dd, 2H, partially obscured by the signal for water), 2.80 (s, 3H), 2.00-1.95 (m, 2H).

HPLC (method 1): Rₜ=2.67 min.

MS (ES+, m/z): 224 (M+H)⁺.

### Example 33A

### 5-Chloro-N-[(2R)-3-{[2-fluoro-4-(3-methyl-2-oxotetrahydropyrimidin-1(2H)-yl)phenyl]amino}-2-hydroxypropyl]thiophene-2-carboxamide

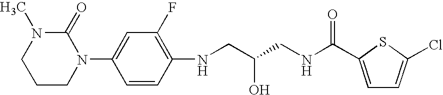

Analogously to the process described under Example 8A, 649 mg (2.91 mmol) of the product from Example 32A and 696 mg (3.198 mmol) of the compound from Example 4A give 1.15 g (90% of theory) of the title compound. The reaction time is 6 hours. Some of the product (895 mg after filtration, washing and drying) precipitates even during the reaction as a solid; the remainder is isolated by preparative HPLC (method 11).

¹H-NMR (400 MHz, DMSO-d₆, δ/ppm): 8.62 (t, 1H), 7.68 (d, 1H), 7.18 (d, 1H), 6.95 (dd, 1H), 6.82 (dd, 1H), 6.67 (dd, 1H), 5.14 (d, 1H), 5.11 (t, 1H), 3.85-3.78 (m, 1H), 3.53 (dd, 2H), 3.37-3.23 (m, 4H, partially obscured by the signal for water), 3.19-3.13 (m, 1H), 3.04-2.98 (m, 1H), 2.82 (s, 3H), 2.02-1.97 (m, 2H).

HPLC (method 1): Rₜ=3.79 min.

MS (ES+, m/z): 441/443 (³⁵Cl/³⁷Cl) (M+H)⁺.

### Example 34A

### 1-(2-{[tert-Butyl(diphenyl)silyl]oxy}ethyl)tetrahydropyrimidin-2(1H)-one

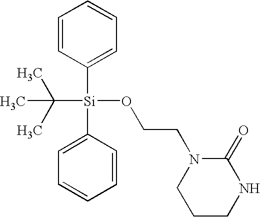

Analogously to the process described under Example 16A, 40.0 g (0.277 mol) of 1-(2-hydroxyethyl)tetrahydropyrimidin-2(1H)-one (CAS No. 53386-63-3) and 92 ml (0.361 mol) of tert-butyldiphenylsilyl chloride are converted into 80.11 g (75% of theory) of the title compound. The product is purified by recrystallization from acetonitrile.

¹H-NMR (400 MHz, DMSO-d₆, δ/ppm): 7.63-7.60 (m, 4H), 7.49-7.41 (m, 6H), 6.17 (s, broad, 1H), 3.69 (t, 2H), 3.35 (t, 2H), 3.29 (t, 2H), 3.10-3.07 (m, 2H), 1.79-1.73 (m, 2H).

HPLC (method 1): Rₜ=5.20 min.

MS (DCl, NH₃, m/z): 383 (M+H)⁺.

### Example 35A

### 1-(2-{[tert-Butyl(diphenyl)silyl]oxy}ethyl)-3-[4-(dibenzylamino)-3-fluorophenyl]tetrahydro-pyrimidin-2(1H)-one

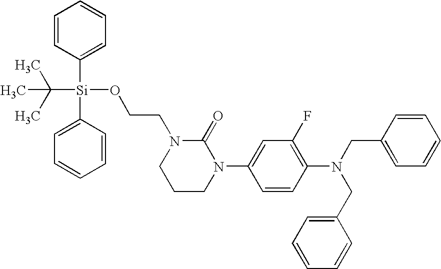

Analogously to the process described in Example 6A, 1.72 g (4.49 mmol) of the compound from Example 34A and 1.5 g (3.59 mmol) of the compound from Example 5A are converted into 1.35 g (56% of theory) of the title compound. The reaction time is 3 days, and the crude product is purified by filtration with suction through silica gel using cyclohexane/ethyl acetate 10:1→2:1.

¹H-NMR (400 MHz, DMSO-d₆, δ/ppm): 7.63-7.61 (m, 4H), 7.47-7.40 (m, 6H), 7.33-7.28 (m, 8H), 7.24-7.19 (m, 2H), 7.09 (dd, 1H), 6.88 (dd, 1H), 6.81 (dd, 1H), 4.27 (s, 4H), 3.75 (dd, 2H), 3.54 (dd, 2H), 3.44-3.40 (m, 4H), 1.98-1.92 (m, 2H), 0.99 (s, 9H).

HPLC (method 2): Rₜ=6.87 min.

MS (DCl, NH₃, m/z): 672 (M+H)⁺, 689 (M+NH₄)⁺.

### Example 36A

### 1-(4-Amino-3-fluorophenyl)-3-(2-{[tert-butyl(diphenyl)silyl]oxy}ethyl)tetrahydropyrimidin-2(1H)-one

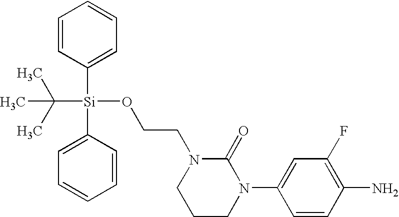

Analogously to the process described under Example 7A, 1.30 g (1.93 mmol) of the compound from Example 35A give 915 mg (96% of theory) of the title compound.

¹H-NMR (400 MHz, DMSO-d₆, δ/ppm): 7.64-7.62 (m, 4H), 7.49-7.41 (m, 6H), 6.88 (dd, 1H), 6.73 (dd, 1H), 6.67 (dd, 1H), 4.97 (s, broad, 2H), 3.75 (dd, 2H), 3.51 (dd, 2H), 3.43-3.40 (m, 4H), 1.99-1.93 (m, 2H), 1.00 (s, 9H).

HPLC (method 2): Rₜ=5.03 min.

MS (ES+, m/z): 492 (M+H)⁺.

### Example 37A

### N-[(2R)-3-({4-[3-(2-{[tert-Butyl(diphenyl)silyl]oxy}ethyl)-2-oxotetrahydropyrimidin-1(2H)-yl]-2-fluorophenyl}amino)-2-hydroxypropyl]-5-chlorothiophene-2-carboxamide

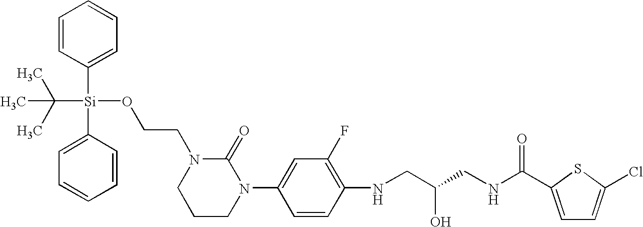

Analogously to the process described under Example 8A, 909 mg (1.85 mmol) of the product from Example 36A and 443 mg (2.03 mmol) of the compound from Example 4A give 770 mg (57% of theory) of the title compound. The reaction time is 40 hours.

¹H-NMR (400 MHz, DMSO-d₆, δ/ppm): 8.61 (t, 1H), 7.68 (d, 1H), 7.64-7.62 (m, 4H), 7.49-7.42 (m, 6H), 7.18 (d, 1H), 6.93 (dd, 1H), 6.82 (dd, 1H), 6.67 (dd, 1H), 5.13 (d, 1H), 5.11 (t, 1H), 3.86-3.79 (m, 1H), 3.76 (dd, 2H), 3.53 (dd, 2H), 3.43-3.41 (m, 4H), 3.38-3.23 (m, 2H, partially obscured by the signal for water), 3.20-3.13 (m, 1H), 3.04-2.99 (m, 1H), 2.00-1.94 (m, 2H), 1.00 (s, 1H).

HPLC (method 2): Rₜ=5.58 min.

MS (ES+, m/z): 709/711 (³⁵Cl/³⁷Cl) (M+H)⁺.

### Example 38A

### N-{[(5S)-3-{4-[3-(2-{[tert-Butyl (diphenyl)silyl]oxy}ethyl)-2-oxotetrahydropyrimidin-1(2H)-yl]-2-fluorophenyl}-2-oxo-1,3-oxazolidin-5-yl]methyl}-5-chlorothiophene-2-carboxamide

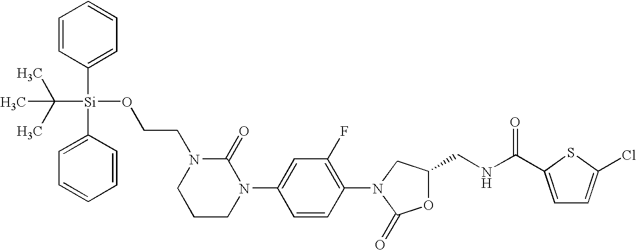

Analogously to the process described under Example 1, 750 mg (1.06 mmol) of the compound from Example 37A and 343 mg (2.12 mmol) of carbonyldiimidazole give 628 mg (81% of theory) of the title compound.

¹H-NMR (400 MHz, DMSO-d₆, δ/ppm): 8.98 (t, 1H), 7.71 (d, 1H), 7.64-7.62 (m, 4H), 7.49-7.42 (m, 6H), 7.38 (dd, 1H), 7.26 (dd, 1H), 7.21 (d, 1H), 7.12 (dd, 1H), 4.89-4.83 (m, 1H), 4.08 (t, 1H), 3.80-3.75 (m, 3H), 3.67-3.55 (m, 4H), 3.48-3.43 (m, 4H), 2.02-1.98 (m, 2H), 1.01 (s, 9H).

HPLC (method 2): Rₜ=5.67 min.

MS (ES+, m/z): 735/737 (³⁵Cl/³⁷Cl) (M+H)⁺.

### Example 39A

### 3-Bromo-1-methylpyrid-2(1H)-one

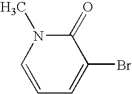

At 40° C., a mixture of 5.0 g (28.7 mmol) of 3-bromo-2-hydroxypyridine, 17.9 ml (0.287 mol) of iodomethane, 1.06 g (2.87 mmol) of tetra-n-butylammonium iodide and 19.9 g (0.144 mol) of potassium carbonate is stirred in 60 ml of toluene for 15 hours. 250 ml of water are then added, and the reaction mixture is extracted with ethyl acetate. The organic extract is washed with saturated sodium chloride solution and dried over anhydrous magnesium sulphate. After filtration and removal of the solvent on a rotary evaporator, the product is isolated by filtration with suction through silica gel using cyclohexane/ethyl acetate 1:1→1:3 as mobile phase. This gives 4.97 g (92% of theory) of the title compound.

¹H-NMR (400 MHz, CDCl₃, δ/ppm): 7.73 (dd, 1H), 7.30 (dd, 1H), 6.06 (dd, 1H), 3.61 (s, 3H).

GC/MS (method 10): Rₜ=5.62 min.

MS (ES+, m/z): 187/189 (⁷⁹Br/⁸¹Br) (M)⁺.

### Example 40A

### O-[tert-Butyl] N-[2-fluoro-4-(1-methyl-2-oxo-1,2-dihydropyridin-3-yl)phenyl]carbamate

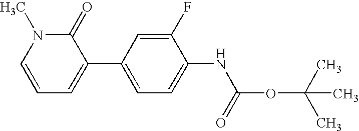

A mixture of 700 mg (3.72 mmol) of the compound from Example 39A, 1044 mg (4.09 mmol) of {4-[(tert-butoxycarbonyl)amino]-3-fluorophenyl}boronic acid, 4.6 ml (9.31 mmol) of 2 molar aqueous sodium carbonate solution and 215 mg (0.186 mmol) of tetrakis(triphenylphosphine)palladium(0) in 15 ml of 1,2-dimethoxyethane is heated at reflux for 15 hours. Water is then added, and the reaction mixture is extracted with ethyl acetate. The organic extract is washed with saturated sodium chloride solution and dried over anhydrous magnesium sulphate. After filtration and removal of the solvent on a rotary evaporator, the product is isolated by filtration with suction through silica gel using cyclohexane/ethyl acetate 5:1 as mobile phase. This gives 933 mg (79% of theory) of the title compound.

MS (DCl, NH₃, m/z): 319 (M+H)⁺, 336 (M+NH₄)⁺.

### Example 41A

### 3-(4-Amino-3-fluorophenyl)-1-methylpyridin-2(1H)-one hydrochloride

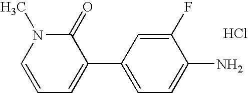

900 mg (2.83 mmol) of the compound from Example 40A are suspended in 75 ml of a 4 molar solution of hydrogen chloride in dioxane. Over time, the starting material dissolves completely. After three hours, all highly volatile components are removed on a rotary evaporator. The residue obtained is suspended in a little dichloromethane, stirred for 30 minutes and then filtered off and dried. This gives 521 mg (72% of theory) of the title compound.

¹H-NMR (500 MHz, DMSO-d₆, δ/ppm): 7.71 (dd, 1H), 7.67-7.61 (m, 2H), 7.41 (dd, 1H), 7.03 (dd, 1H), 6.30 (dd, 1H), 3.49 (s, 3H).

LC/MS (method 5): Rₜ=1.33 min.

MS (ES+, m/z): 219 (M+H)⁺.

### Example 42A

### 5-Chloro-N-[(2R)-3-{[2-fluoro-4-(1-methyl-2-oxo-1,2-dihydropyridin-3-yl)phenyl]amino}-2-hydroxypropyl]thiophene-2-carboxamide

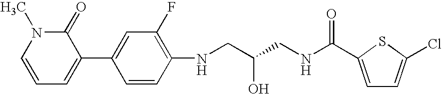

Initially, 400 mg (1.57 mmol) of the hydrochloride from Example 41A are converted into the free base by dissolving the hydrochloride in 200 ml of saturated sodium bicarbonate solution, followed by extraction with ethyl acetate. The organic extract is dried over anhydrous magnesium sulphate, filtered and freed from the solvent on a rotary evaporator. The aniline obtained in this manner is reacted analogously to the process described under Example 8A with 376 mg (1.73 mmol) of the compound from Example 4A, to give 381 mg (56% of theory) of the title compound.

¹H-NMR (400 MHz, DMSO-d₆, δ/ppm): 8.64 (t, 1H), 7.69 (d, 1H), 7.65 (dd, 1H), 7.59 (dd, 1H), 7.57 (dd, 1H), 7.39 (dd, 1H), 7.19 (d, 1H), 6.76 (dd, 1H), 6.28 (dd, 1H), 5.41 (t, 1H), 5.18 (d, 1H), 3.88-3.80 (m, 1H), 3.49 (s, 3H), 3.40-3.24 (m, 2H, partially obscured by the signal for water), 3.23-3.18 (m, 1H), 3.11-3.04 (m, 1H).

LC/MS (method 3): Rₜ=1.85 min.

MS (ES+, m/z): 436/438 (³⁵Cl/³⁷Cl) (M+H)⁺.

### Example 43A

### O-[tert-Butyl] N-[2-fluoro-4-(2-hydroxypyridin-3-yl)phenyl]carbamate

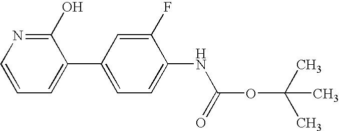

Analogously to the process described under Example 40A, 618 mg (3.55 mmol) of 3-bromo-2-hydroxypyridine and 996 mg (3.91 mmol) of {4-[(tert-butoxycarbonyl)amino]-3-fluorophenyl}-boronic acid are converted into 179 mg (17% of theory) of the title compound. The product is precipitated by addition of water and a little ethyl acetate to the reaction mixture, filtered, washed and dried.

¹H-NMR (400 MHz, DMSO-d₆, δ/ppm): 11.83 (s, broad, 1H), 8.97 (s, 1H), 7.71 (dd, 1H), 7.70 (dd, 1H), 7.60 (dd, 1H), 7.50 (dd, 1H), 7.39 (dd, 1H), 6.30 (dd, 1H), 1.47 (s, 9H).

LC/MS (method 1): Rₜ=4.06 min.

MS (ES+, m/z): 305 (M+H)⁺.

### Example 44A

### 3-(4-Amino-3-fluorophenyl)pyridin-2-ol hydrochloride

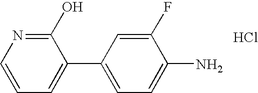

Analogously to the process described under Example 41A, 420 mg (1.38 mmol) of the compound from Example 43A are converted into 255 mg (82% of theory) of the title compound.

¹H-NMR (400 MHz, DMSO-d₆, δ/ppm): 11.78 (broad, 1H), 7.70 (dd, 1H), 7.65 (dd, 1H), 7.44 (dd, 1H), 7.34 (dd, 1H), 7.02 (dd, 1H), 6.27 (dd, 1H).

LC/MS (method 6): Rₜ=2.34 min.

MS (ES+, m/z): 205 (M+H)⁺.

### Example 45A

### 5-Chloro-N-[(2R)-3-{[2-fluoro-4-(2-hydroxypyridin-3-yl)phenyl]amino}-2-hydroxypropyl]thiophene-2-carboxamide

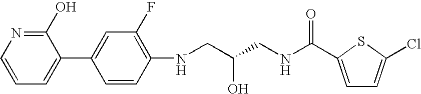

Initially, 109 mg (0.453 mmol) of the hydrochloride from Example 44A are converted into the free base as described in Example 42A. The aniline obtained in this manner is then reacted analogously to the process described under Example 8A with 109 mg (0.498 mmol) of the compound from Example 4A, to give 110 mg (57% of theory) of the title compound

¹H-NMR (400 MHz, DMSO-d₆, δ/ppm): 11.66 (s, broad, 1H), 8.61 (t, 1H), 7.67 (d, 1H), 7.62 (dd, 1H), 7.59 (dd, 1H), 7.41 (dd, 1H), 7.29 (dd, 1H), 7.17 (d, 1H), 6.73 (dd, 1H), 6.23 (dd, 1H), 5.38 (t, 1H), 5.16 (d, 1H), 3.88-3.80 (m, 1H), 3.39-3.24 (m, 2H, partially obscured by the signal for water), 3.23-3.18 (m, 1H), 3.10-3.03 (m, 1H).

HPLC (method 1): Rₜ=3.77 min.

MS (ES+, m/z): 422/424 (³⁵Cl/³⁷Cl) (M+H)⁺.

### Example 46A

### 1-(4-Amino-3-fluorophenyl)pyridin-2(1H)-one

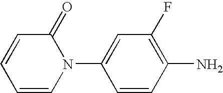

Analogously to the process described under Example 6A, 1000.0 mg (4.22 mmol) of 2-fluoro-4-iodoaniline and 502 mg (5.27 mmol) of 2-hydroxypyridine are converted into 817 mg (95% of theory) of the title compound. The crude product is purified by filtration with suction through silica gel using dichloromethane/methanol 10:1.

¹H-NMR (500 MHz, DMSO-d₆, δ/ppm): 7.57 (dd, 1H), 7.45 (dd, 1H), 7.10 (dd, 1H), 6.89 (dd, 1H), 6.81 (dd, 1H), 6.43 (d, 1H), 6.26 (dd, 1H), 5.40 (s, broad, 2H).

HPLC (method 1): Rₜ=2.47 min.

MS (ES+, m/z): 205 (M+H)⁺.

### Example 47A

### 5-Chloro-N-[(2R)-3-{[2-fluoro-4-(2-oxopyridin-1(2H)-yl)phenyl]amino}-2-hydroxypropyl]thiophene-2-carboxamide

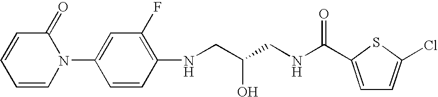

Analogously to the process described under Example 8A, 800 mg (3.92 mmol) of the product from Example 46A and 938 mg (4.31 mmol) of the compound from Example 4A give 770 mg (47% of theory) of the title compound.

¹H-NMR (400 MHz, DMSO-d₆, δ/ppm): 8.62 (t, 1H), 7.69 (d, 1H), 7.57 (dd, 1H), 7.47 (dd, 1H), 7.18 (d, 1H), 7.17 (dd, 1H), 6.99 (dd, 1H), 6.82 (dd, 1H), 6.43 (d, 1H), 6.26 (dd, 1H), 5.55 (t, 1H), 5.17 (d, 1H), 3.89-3.81 (m, 1H), 3.40-3.33 (m, 1H), 3.30-3.19 (m, 2H, partially obscured by the signal for water), 3.12-3.07 (m, 1H).

HPLC (method 1): Rₜ=3.84 min.

MS (DCl, NH₃, m/z): 422/424 (³⁵Cl/³⁷Cl) (M+H)⁺, 439/441 (M+NH₄)⁺.

### Example 48A

### 1-(4-Amino-3-fluorophenyl)-3-hydroxypyridin-2(1H)-one

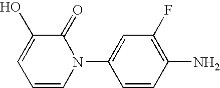

Analogously to the process described under Example 6A, 1000.0 mg (4.22 mmol) of 2-fluoro-4-iodoaniline and 586 mg (5.27 mmol) of 2,3-dihydroxypyridine are converted into 290 mg (31% of theory) of the title compound. The crude product is purified by filtration with suction through silica gel using dichloromethane/methanol 50:0→50:1, which also results in the recovery of 163 mg (35% of theory) of the 2,3-dihydroxypyridine.

¹H-NMR (400 MHz, DMSO-d₆, δ/ppm): 9.09 (s, 1H), 7.12 (dd, 1H), 7.03 (dd, 1H), 6.90 (dd, 1H), 6.82 (dd, 1H), 6.74 (dd, 1H), 6.14 (dd, 1H), 5.39 (s, 2H).

HPLC (method 1): Rₜ=2.56 min.

MS (ES+, m/z): 221 (M+H)⁺.

### Example 49A

### 1-(4-Amino-3-fluorophenyl)-3-(2-{[tert-butyl(dimethyl)silyl]oxy}ethoxy)pyridin-2(1H)-one

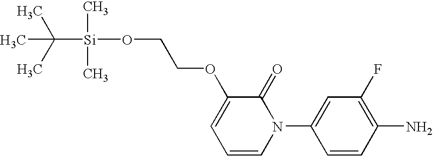

359 mg (2.60 mmol) of potassium carbonate are added to a solution of 286 mg (1.30 mmol) of the product from Example 48A in 5 ml of anhydrous DMF, and the mixture is stirred at room temperature for 30 minutes. 418 μl (1.95 mmol) of (2-bromoethoxy)-tert-butyldimethylsilane are then added. The reaction mixture is stirred at 60° C. for five hours. After cooling, 20 ml of water are added and the mixture is extracted with ethyl acetate. The organic extract is washed with water and saturated sodium chloride solution. After drying over anhydrous magnesium sulphate and filtration, the solvent is removed on a rotary evaporator. The product is purified by flash chromatography on silica gel using dichloromethane/methanol 50:0→50:1 as mobile phase. This gives 379 mg (76% of theory) of the title compound.

¹H-NMR (400 MHz, DMSO-d₆, δ/ppm): 7.13 (dd, 1H), 7.07 (dd, 1H), 6.87-6.79 (m, 3H), 6.15 (dd, 1H), 5.39 (s, 2H), 3.96 (t, 2H), 3.91 (t, 2H), 0.86 (s, 9H), 0.07 (s, 6H).

LC/MS (method 8): Rₜ=3.57 min.

MS (ES+, m/z): 379 (M+H)⁺.

### Example 50A

### N-[(2R)-3-({4-[3-(2-{[tert-Butyl(dimethyl)silyl]oxy}ethoxy)-2-oxopyridin-1(2H)-yl]-2-fluorophenyl}amino)-2-hydroxypropyl]-5-chlorothiophene-2-carboxamide

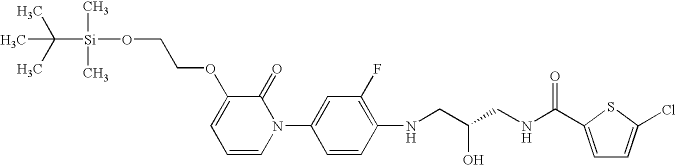

Analogously to the process described under Example 8A, 358 mg (0.946 mmol) of the product from Example 49A and 227 mg (1.04 mmol) of the compound from Example 4A give 168 mg (30% of theory) of the title compound.

¹H-NMR (400 MHz, DMSO-d₆, δ/ppm): 8.64 (t, 1H), 7.68 (d, 1H), 7.18 (d, 1H), 7.15 (dd, 1H), 7.13 (dd, 1H), 6.97 (dd, 1H), 6.88 (dd, 1H), 6.81 (dd, 1H), 6.15 (dd, 1H), 5.57 (t, 1H), 5.19 (d, 1H), 3.97-3.89 (m, 4H), 3.88-3.82 (m, 1H), 3.40-3.19 (m, 4H, partially obscured by the signal for water), 3.12-3.06 (m, 1H), 0.87 (s, 9H), 0.08 (s, 6H).

LC/MS (method 8): Rₜ=3.88 min.

MS (EI+, m/z): 596/598 (³⁵Cl/³⁷Cl) (M+H)⁺.

### Example 51A

### N-{[(5S)-3-{4-[3-(2-{[tert-Butyl(dimethyl)silyl]oxy}ethoxy)-2-oxopyridin-1(2H)-yl]-2-fluorophenyl}-2-oxo-1,3-oxazolidin-5-yl]methyl}-5-chlorothiophene-2-carboxamide

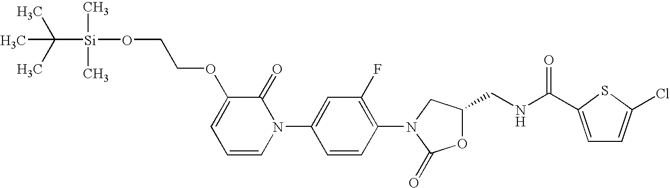

Analogously to the process described under Example 1, 165 mg (0.277 mmol) of the compound from Example 50A and 90 mg (0.554 mmol) of carbonyldiimidazole give 63 mg (36% of theory) of the title compound. The reaction time is 40 hours.

¹H-NMR (400 MHz, DMSO-d₆, δ/ppm): 8.99 (t, 1H), 7.71 (d, 1H), 7.63 (dd, 1H), 7.50 (dd, 1H), 7.29 (dd, 1H), 7.23 (dd, 1H), 7.20 (d, 1H), 6.91 (dd, 1H), 6.23 (dd, 1H), 4.93-4.87 (m, 1H), 4.17 (t, 1H), 3.98 (t, 2H), 3.91 (t, 2H), 3.86 (dd, 1H), 3.66-3.62 (m, 2H), 0.86 (s, 9H), 0.08 (s, 6H).

LC/MS (method 8): Rₜ=3.87 min.

MS (ES+, m/z): 622/624 (³⁵Cl/³⁷Cl) (M+H)⁺.

### Example 52A

### 3-Bromo-1-(3-fluoro-4-nitrophenyl)pyridin-2(1H)-one

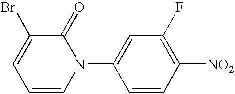

At 0° C., 1.94 g (17.2 mmol) of potassium tert-butoxide are added to a solution of 2.5 g (14.4 mmol) of 3-bromo-2-hydroxypyridine in 30 ml of anhydrous DMF, and the mixture is stirred at room temperature for 45 minutes. After this period, a solution of 2.51 g (15.8 mmol) of 2,4-difluoronitrobenzene in 10 ml of anhydrous DMF is added dropwise to the reaction mixture. Stirring at room temperature is continued for 15 hours. 120 ml of water are then added, and the mixture is extracted with ethyl acetate. The organic extract is washed with water and saturated sodium chloride solution. After drying over anhydrous sodium sulphate, the mixture is filtered and the filtrate is freed from the solvent on a rotary evaporator. The crude product is initially freed from coarse impurities by filtration with suction through silica gel using cyclohexane/ethyl acetate 5:1→1:1 as mobile phase. The product is then isolated by preparative HPLC. To this end, 2.1 g of the crude product obtained are dissolved in 5 ml of acetonitrile and chromatographed in 10 portions.

Chromatography: column: Kromasil 100C18, 5 μm, 250×20 mm; flow rate: 25 ml/min; temperature: 40° C.; UV detection: 210 nm; mobile phase: water/acetonitrile 68:32.

This gives 367 mg (8% of theory) of the title compound.

¹H-NMR (400 MHz, DMSO-d₆, δ/ppm): 8.31 (dd, 1H), 8.07 (dd, 1H), 7.93 (dd, 1H), 7.80 (dd, 1H), 7.61 (dd, 1H), 6.34 (dd, 1H).

LC/MS (method 4): Rₜ=1.93 min.

MS (ES+, m/z): 313/315 (⁷⁹Br/⁸¹Br) (M+H)⁺.

### Example 53A

### 3-Allyl-1-(3-fluoro-4-nitrophenyl)pyridin-2-(1H)-one

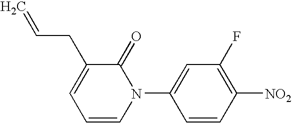

323 μl (1.73 mmol) of 2-allyl-4,4,5,5-tetramethyl-1,3,2-dioxaborolane are added dropwise to a mixture of 360 mg (1.15 mmol) of the compound from Example 52A, 349 mg (2.30 mmol) of caesium fluoride and 42 mg (0.057 mmol) of [1,1′-bis(diphenylphosphino)ferrocene]palladium(II) dichloride in 6.6 ml of anhydrous 1,2-dimethoxyethane. The reaction mixture is then heated at 80° C. for 15 hours. After cooling, saturated sodium bicarbonate solution is added and the mixture is extracted with ethyl acetate. The organic extract is dried over anhydrous magnesium sulphate. After filtration and evaporation of the filtrate on a rotary evaporator, the product is isolated by preparative HPLC (method 11). This gives 243 mg (77% of theory) of the title compound.

¹H-NMR (400 MHz, DMSO-d₆, δ/ppm): 8.29 (dd, 1H), 7.87 (dd, 1H), 7.62 (dd, 1H), 7.57 (dd, 1H), 7.37 (dd, 1H), 6.35 (dd, 1H), 6.01-5.90 (m, 1H), 5.16-5.07 (m, 2H), 3.20 (d, 2H).

HPLC (method 1): Rₜ=4.13 min.

MS (DCl, NH₃, m/z): 275 (M+H)⁺, 292 (M+NH₄)⁺.

### Example 54A

### 3-Allyl-1-(4-amino-3-fluorophenyl)pyridin-2(1H)-one

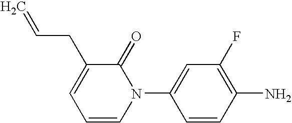

A mixture of 237 mg (0.864 mmol) of the compound from Example 53A and 975 mg (4.32 mmol) of tin(II) chloride dihydrate in 10 ml of methanol is heated at reflux for two hours. 250 ml of water are then added, and the mixture is made alkaline using 1 molar aqueous sodium hydroxide solution and extracted with ethyl acetate. The organic extract is washed successively with water and saturated sodium chloride solution. Drying over anhydrous magnesium sulphate, filtration and removal of the solvent on a rotary evaporator give 215 mg (97% of theory) of the title compound.

¹H-NMR (400 MHz, DMSO-d₆, δ/ppm): 7.45 (dd, 1H), 7.28 (dd, 1H), 7.09 (dd, 1H), 6.88 (dd, 1H), 6.80 (dd, 1H), 6.21 (dd, 1H), 6.00-5.89 (m, 1H), 5.35 (s, broad, 2H), 5.12-5.03 (m, 2H), 3.17 (d, 2H).

LC/MS (method 3): Rₜ=1.61 min.

MS (ES+, m/z): 245 (M+H)⁺.

### Example 55A

### N-[(2R)-3-{[4-(3-Allyl-2-oxopyridin-1(2H)-yl)-2-fluorophenyl]amino}-2-hydroxypropyl]-5-chlorothiophene-2-carboxamide

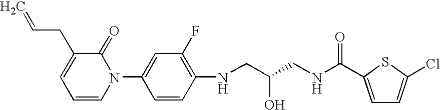

Analogously to the process described under Example 8A, 213 mg (0.872 mmol) of the product from Example 54A and 209 mg (0.959 mmol) of the compound from Example 4A give 224 mg (56% of theory) of the title compound. The reaction time is 40 hours.

¹H-NMR (400 MHz, DMSO-d₆, δ/ppm): 8.65 (t, 1H), 7.68 (d, 1H), 7.48 (dd, 1H), 7.29 (dd, 1H), 7.18 (d, 1H), 7.16 (dd, 1H), 6.98 (dd, 1H), 6.81 (dd, 1H), 6.22 (dd, 1H), 6.00-5.90 (m, 1H), 5.56 (t, 1H), 5.19 (d, 1H), 5.13-5.04 (m, 2H), 3.88-3.81 (m, 1H), 3.40-3.17 (m, 3H, partially obscured by the signal for water), 3.18 (d, 2H), 3.12-3.07 (m, 1H).

HPLC (method 1): Rₜ=4.27 min.

MS (DCl, NH₃, m/z): 462/464 (³⁵Cl/³⁷Cl) (M+H)⁺479/481 (M+NH₄)⁺.

### Example 56A

### N-({(5S)-3-[4-(3-Allyl-2-oxopyridin-1(2H)-yl)-2-fluorophenyl]-2-oxo-1,3-oxazolidin-5-yl}methyl)-5-chlorothiophene-2-carboxamide

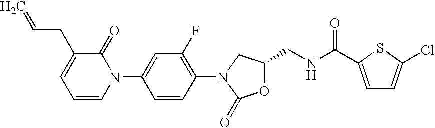

Analogously to the process described under Example 1, 220 mg (0.476 mmol) of the compound from Example 55A and 154 mg (0.952 mmol) of carbonyldiimidazole give 120 mg (52% of theory) of the title compound.

¹H-NMR (400 MHz, DMSO-d₆, δ/ppm): 9.00 (t, 1H), 7.71 (d, 1H), 7.63 (dd, 1H), 7.58 (dd, 1H), 7.52 (dd, 1H), 7.34-7.30 (m, 2H), 7.20 (d, 1H), 6.30 (dd, 1H), 6.00-5.90 (m, 1H), 5.15-5.06 (m, 2H), 4.93-4.87 (m, 1H), 4.18 (t, 1H), 3.86 (dd, 1H), 3.68-3.59 (m, 2H), 3.19 (d, 2H).

LC/MS (method 4): Rₜ=2.24 min.

MS (ES+, m/z): 488/490 (³⁵Cl/³⁷Cl) (M+H)⁺.

### Example 57A

### 3-Methyl-1-(3-chloro-4-nitrophenyl)pyridin-2(1H)-one

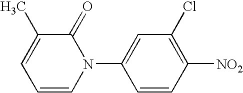

Analogously to the process described under Example 52A, 500 mg (4.58 mmol) of 2-hydroxy-3-methylpyridine and 885 mg (5.04 mmol) of 2-chloro-4-fluoronitrobenzene give 780 mg (63% of theory) of the title compound. The reaction time is two hours. The product is isolated by flash chromatography on silica gel using cyclohexane/ethyl acetate 2:1 as mobile phase.

¹H-NMR (300 MHz, DMSO-d₆, δ/ppm): 8.23 (d, 1H), 7.99 (d, 1H), 7.70 (dd, 1H), 7.60 (dd, 1H), 7.43 (dd, 1H), 6.30 (dd, 1H), 2.04 (s, 3H).

HPLC (method 2): Rₜ=4.08 min.

MS (DCl, NH₃, m/z): 265/267 (³⁵Cl/³⁷Cl) (M+H)⁺, 282/284 (M+NH₄⁺).

### Example 58A

### 3-Methyl-1-(4-amino-3-chlorophenyl)pyridin-2(1H)-one

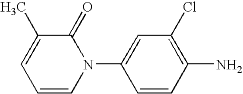

Analogously to the process described in Example 54A, reduction of 250 mg (0.94 mmol) of the product from Example 57A gives 252 mg (97% of theory) of the title compound. The reaction is carried out in ethanol.

¹H-NMR (400 MHz, DMSO-d₆, δ/ppm): 7.42 (dd, 1H), 7.35 (dd, 1H), 7.23 (d, 1H), 7.02 (dd, 1H), 6.83 (d, 1H), 6.17 (dd, 1H), 5.59 (s, broad, 2H), 2.01 (s, 3H).

HPLC (method 1): Rₜ=3.62 min.

MS (DCl, NH₃, m/z): 235/237 (³⁵Cl/³⁷Cl) (M+H)⁺, 252/254 (M+NH₄⁺).

### Example 59A

### 2-[(2S)-Oxiran-2-ylmethyl]-1H-isoindole-1,3(2H)-dione

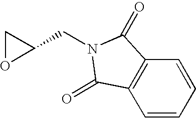

The title compound is prepared analogously to a process known from the literature [A. Gutcait et al. Tetrahedron Asym. 1996, 7, 1641].

### Example 60A

### 2-[(2R)-3-{[2-Fluoro-4-(3-oxomorpholin-4-yl)phenyl]amino}-2-hydroxypropyl]-1H-isoindole-1,3(2H)-dione

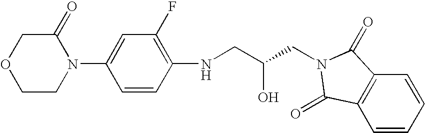

A solution of 24.4 g (116 mmol) of the compound from Example 7A and 23.5 g (116 mmol, 1 eq.) of the compound from Example 59A in 500 ml of a 9:1 mixture of ethanol and water is stirred at 75° C. overnight. Extra portions of 7.1 g (35 mmol, 0.3 eq.), 3.5 g (17 mmol, 0.15 eq.) and 4.7 g (23 mmol, 0.2 eq.) of the compound from Example 59A are added, and after each addition the reaction mixture is stirred at 75° C. overnight. The reaction mixture is concentrated under reduced pressure. The residue is triturated with acetonitrile, filtered and dried under reduced pressure, giving 21.4 g (43% of theory) of the title compound. The combined mother liquors are concentrated under reduced pressure, and the residue is purified by flash chromatography (silica gel 60, dichloromethane/methanol 100:1→100:2). This gives a further 7.1 g (14% of theory) of the title compound.

LC-MS (method 8): Rₜ=2.18 min;

MS (ESIpos): m/z=414 [M+H]⁺.

### Example 61A

### 2-({(5S)-3-[2-Fluoro-4-(3-oxomorpholin-4-yl)phenyl]-2-oxo-1,3-oxazolidin-5-yl}methyl)-1H-isoindole-1,3(2H)-dione

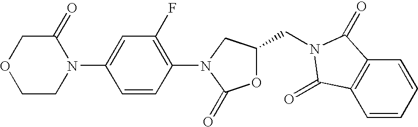

A solution of 21.4 g (52 mmol) of the compound from Example 60A, 12.6 g (78 mmol, 1.5 eq.) of 1,1′-carbonyldiimidazole and 3.2 g (26 mmol, 0.5 eq.) of 4-dimethylaminopyridine in 750 ml of tetrahydrofuran is stirred at 60° C. overnight. After cooling of the reaction mixture, the precipitate formed (desired product) is filtered off and dried under reduced pressure; a further 1.3 g (10 mmol, 0.2 eq.) of 4-dimethylaminopyridine are added to the filtrate, which is stirred at 60° C. for a further night. These steps are repeated another three times, giving a total of 17 g (73% of theory) of the title compound. The last filtrate is concentrated under reduced pressure and the residue is triturated with acetonitrile, filtered and dried under reduced pressure, which gives a further 5.9 g (25% of theory) of the title compound.

LC-MS (method 8): Rₜ=2.20 min;

MS (ESIpos): m/z=440 [M+H]⁺.

### Example 62A

### 4-{4-[(5S)-5-(Aminomethyl)-2-oxo-1,3-oxazolidin-3-yl]-3-fluorophenyl}morpholin-3-one

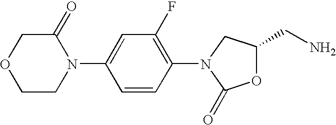

43 ml of methylamine (40% strength in water, 498 mmol, 14 eq.) are added to a solution of 16.2 g (37 mmol) of the compound from Example 61A in 220 ml of ethanol, and the mixture is stirred under reflux for 45 min. The reaction mixture is concentrated under reduced pressure, and the residue is triturated with acetonitrile, filtered and dried under reduced pressure. This gives 12 g (95% of theory) of the title compound.

LC-MS (method 6): Rₜ=1.70 min;

MS (ESIpos): m/z=310 [M+H]⁺.

### WORKING EXAMPLES

### Example 1

### 5-Chloro-N-({(5S)-3-[2-fluoro-4-(3-oxomorpholin-4-yl)phenyl]-2-oxo-1,3-oxazolidin-5-yl}methyl)thiophene-2-carboxamide

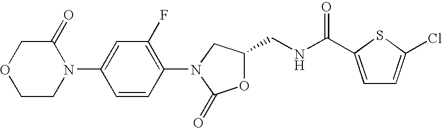

### Method 1:

2.7 mg (0.022 mmol) of 4-dimethylaminopyridine are added to a solution of 478 mg (1.12 mmol) of the product from Example 8A and 363 mg (2.24 mmol) of carbonyldiimidazole in 10 ml of butyronitrile, and the mixture is heated at 70° C. After three days, the solvent is removed on a rotary evaporator. The product is isolated from the residue by preparative HPLC (method 11). This gives 344 mg (68% of theory) of the title compound.

¹H-NMR (400 MHz, DMSO-d₆, δ/ppm): 8.98 (t, 1H), 7.70 (d, 1H), 7.52 (dd, 1H), 7.48 (dd, 1H), 7.31 (dd, 1H), 7.21 (d, 1H), 4.91-4.84 (m, 1H), 4.21 (s, 2H), 4.12 (t, 1H), 3.98 (dd, 2H), 3.80 (dd, 1H), 3.76 (dd, 2H), 3.68-3.57 (m, 2H).

HPLC (method 1): Rₜ=3.82 min.

MS (DCl, NH₃, m/z): 471/473 (³⁵Cl/³⁷Cl) (M+NH₄)⁺.

### Method 2:

At 0° C., 7.9 g (43 mmol, 1.2 eq.) of the compound from Example 1A are added to a solution of 11.2 g (36 mmol) of the compound from Example 62A in 224 ml of pyridine. After 30 min, the reaction mixture is concentrated under reduced pressure and the residue is taken up in water and dichloromethane. After phase separation, the aqueous phase is extracted twice with dichloromethane. The combined organic phases are washed with water and with saturated aqueous sodium chloride solution, dried over sodium sulphate, filtered and concentrated under reduced pressure. The residue is triturated with dichloromethane, filtered and dried under reduced pressure, which gives 7.4 g (45% of theory) of the title compound. The filtrate is concentrated under reduced pressure and the residue is purified by flash chromatography (silica gel 60, dichloromethane/methanol 100:1→100:2), which gives a further 1.9 g (12% of theory) of the title compound.

HPLC (method 2): Rₜ=3.74 min;

MS (ESIpos): m/z=454 [M+H]⁺;

¹H-NMR (500 MHz, DMSO-d₆): δ=8.94 (t, 1H), 7.69 (d, 1H), 7.52 (dd, 1H), 7.48 (dd, 1H), 7.31 (dd, 1H), 7.20 (d, 1H), 4.92-4.84 (m, 1H), 4.21 (s, 2H), 4.12 (t, 1H), 3.97 (t, 2H), 3.81 (dd, 1H), 3.76 (t, 2H), 3.67-3.56 (m, 2H);

melting points: 177° C., ΔH 84 Jg⁻¹ and 183° C., ΔH 7 Jg⁻¹.

### Example 2

### 5-Chloro-N-({(5S)-3-[2-fluoro-4-(3-hydroxy-2-oxopiperidin-1-yl)phenyl]-2-oxo-1,3-oxazolidin-5-yl}methyl)thiophene-2-carboxamide (Mixture of diastereomers)

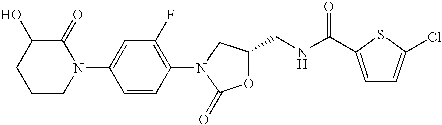

At 0° C., 1.17 ml (1.17 mmol) of a 1 molar solution of tetra-n-butylammonium fluoride in THF are added to a solution of 648 mg (1.11 mmol) of the compound from Example 12A in 20 ml of THF. After one hour at room temperature, the reaction mixture is diluted with water and extracted with ethyl acetate. The organic extract is washed successively with water and saturated sodium chloride solution. After drying over anhydrous magnesium sulphate, filtration and concentration using a rotary evaporator, the crude product obtained is purified by preparative HPLC (method 11). This gives 421 mg (81% of theory) of the title compound.

¹H-NMR (400 MHz, DMSO-d₆, δ/ppm): 8.98 (t, 1H), 7.70 (d, 1H), 7.49 (dd, 1H), 7.33 (dd, 1H), 7.20 (d, 1H), 7.18 (dd, 1H), 5.32 (d, 1H), 4.90-4.84 (m, 1H), 4.14-4.05 (m, 2H), 3.80 (dd, 2H), 3.72-3.66 (m, 1H), 3.63-3.60 (m, 2H), 3.58-3.52 (m, 1H), 2.13-2.06 (m, 1H), 1.99-1.83 (m, 2H), 1.79-1.69 (m, 1H).

HPLC (method 1): Rₜ=3.76 min.

MS (DCl, NH₃, m/z): 485/487 (³⁵Cl/³⁷Cl) (M+NH₄)⁺.

### Example 3

### 5-Chloro-N-({(5S)-3-[2-fluoro-4-(3-hydroxy-2-oxopiperidin-1-yl)phenyl]-2-oxo-1,3-oxazolidin-5-yl}methyl)thiophene-2-carboxamide (diastereomer 1)

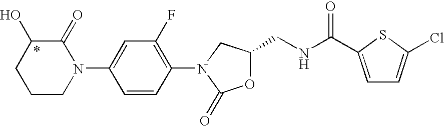

On a preparative scale, the mixture of diastereomers from Example 2 is separated chromatographically into the pure diastereomers. To this end, 390 mg of the compound from Example 2 are dissolved in 30 ml of the mobile phase and chromatographed in 75 portions. This gives 161 mg (41% of theory) of the title compound (diastereomer 1) and 169 mg (43% of theory) of diastereomer 2.

Method: column: Daicel Chiralpak IA-H, 5 μm, 250 mm×20 mm; flow rate: 15 ml/min; temperature: 30° C.; UV detection: 220 nm; mobile phase: tert-butyl methyl ether/methanol 1:1.

Retention time: 7.28 min (diastereomer 1), 8.20 min (diastereomer 2)

¹H-NMR (400 MHz, DMSO-d₆, δ/ppm): 8.98 (t, 1H), 7.70 (d, 1H), 7.49 (dd, 1H), 7.33 (dd, 1H), 7.20 (d, 1H), 7.19 (dd, 1H), 5.32 (d, 1H), 4.90-4.83 (m, 1H), 4.13-4.05 (m, 2H), 3.80 (dd, 2H), 3.72-3.66 (m, 1H), 3.63-3.52 (m, 3H), 2.12-2.06 (m, 1H), 2.00-1.82 (m, 2H), 1.78-1.69 (m, 1H).

HPLC (method 1): Rₜ=3.72 min.

MS (ESIpos, m/z): 468/470 (³⁵Cl/³⁷Cl) (M+H)⁺.

### Example 4

### 5-Chloro-N-({(5S)-3-[2-fluoro-4-(3-hydroxy-2-oxopiperidin-1-yl)phenyl]-2-oxo-1,3-oxazolidin-5-yl}methyl)thiophene-2-carboxamide (diastereomer 2)

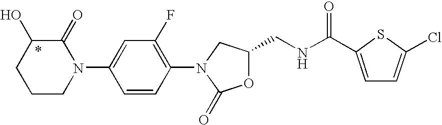

On a preparative scale, the mixture of diastereomers from Example 2 is separated chromatographically into the pure diastereomers. To this end, 390 mg of the compound from Example 2 are dissolved in 30 ml of the mobile phase and chromatographed in 75 portions. This gives 169 mg (43% of theory) of the title compound (diastereomer 2) and 161 mg (43% of theory) of diastereomer 1.

Method: column: Daicel Chiralpak IA-H, 5 μm, 250 mm×20 mm; flow rate: 15 ml/min; temperature: 30° C.; UV detection: 220 nm; mobile phase: tert-butyl methyl ether/methanol 1:1.

Retention time: 7.28 min (diastereomer 1), 8.20 min (diastereomer 2)

¹H-NMR (400 MHz, DMSO-d₆, δ/ppm): 8.98 (t, 1H), 7.71 (d, 1H), 7.48 (dd, 1H), 7.33 (dd, 1H), 7.21 (d, 1H), 7.19 (dd, 1H), 5.33 (d, 1H), 4.90-4.84 (m, 1H), 4.14-4.05 (m, 2H), 3.80 (dd, 2H), 3.72-3.67 (m, 1H), 3.63-3.52 (m, 3H), 2.13-2.06 (m, 1H), 2.00-1.82 (m, 2H), 1.79-1.70 (m, 1H).

HPLC (method 1): Rₜ=3.72 min.

MS (ESIpos, m/z): 468/470 (³⁵Cl/³⁷Cl) (M+H)⁺.

### Example 5

### 5-Chloro-N-({(5S)-3-[2-fluoro-4-(1-methyl-2-oxopiperidin-3-yl)phenyl]-2-oxo-1,3-oxazolidin-5-yl}methyl)thiophene-2-carboxamide (Mixture of diastereomers)

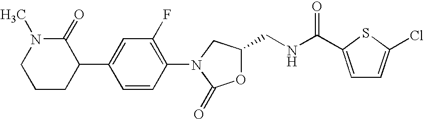

Analogously to the process described under Example 1, 730 mg (1.66 mmol) of the compound from Example 15A and 538 mg (3.32 mmol) of carbonyldiimidazole give 630 mg (81% of theory) of the title compound. The reaction time is 15 hours.

¹H-NMR (400 MHz, DMSO-d₆, δ/ppm): 8.97 (t, 1H), 7.70 (d, 1H), 7.38 (dd, 1H), 7.20 (d, 1H), 7.12 (dd, 1H), 7.04 (dd, 1H), 4.89-4.83 (m, 1H), 4.12-4.07 (m, 1H), 3.78 (dd, 1H), 3.66-3.54 (m, 3H), 3.46-3.39 (m, 1H), 3.33-3.28 (m, 1H, partially obscured by the signal for water), 2.86 (s, 3H), 2.07-2.00 (m, 1H), 1.93-1.77 (m, 3H).

HPLC (method 1): Rₜ=3.98 min.

MS (DCl, NH₃, m/z): 483/485 (³⁵Cl/³⁷Cl) (M+NH₄)⁺.

### Example 6

### 5-Chloro-N-({(5S)-3-[2-fluoro-4-(1-methyl-2-oxopiperidin-3-yl)phenyl]-2-oxo-1,3-oxazolidin-5-yl}methyl)thiophene-2-carboxamide (diastereomer 1)

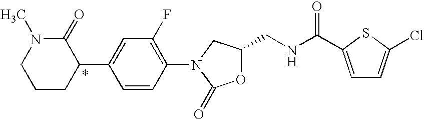

On a preparative scale, the mixture of diastereomers from Example 5 is separated chromatographically into the pure diastereomers. To this end, 432 mg of the compound from Example 5 are dissolved in a mixture of 10 ml of methanol, 10 ml of tert-butyl methyl ether and 5 ml of acetonitrile and chromatographed in ten portions. This gives 182 mg (42% of theory) of the title compound (diastereomer 1) and 156 mg (36% of theory) of diastereomer 2.

Method: column: Daicel Chiralpak IA-H, 5 μm, 250 mm×20 mm; flow rate: 15 ml/min; temperature: 30° C.; UV detection: 220 nm; mobile phase: tert-butyl methyl ether/methanol 1:1.

Retention time: 5.91 min (diastereomer 1), 8.81 min (diastereomer 2)

¹H-NMR (500 MHz, DMSO-d₆, δ/ppm): 8.99 (t, 1H), 7.70 (d, 1H), 7.37 (dd, 1H), 7.20 (d, 1H), 7.13 (dd, 1H), 7.03 (dd, 1H), 4.89-4.83 (m, 1H), 4.08 (t, 1H), 3.78 (dd, 1H), 3.65-3.56 (m, 3H), 3.44-3.39 (m, 1H), 3.33-3.29 (m, 1H, partially obscured by the signal for water), 2.87 (s, 3H), 2.06-2.00 (m, 1H), 1.92-1.76 (m, 3H).

HPLC (method 1): Rₜ=3.92 min.

MS (DCl, NH₃, m/z): 483/485 (³⁵Cl/³⁷Cl) (M+NH₄)⁺.

### Example 7

### 5-Chloro-N-({(5S)-3-[2-fluoro-4-(1-methyl-2-oxopiperidin-3-yl)phenyl]-2-oxo-1,3-oxazolidin-5-yl}methyl)thiophene-2-carboxamide (diastereomer 2)

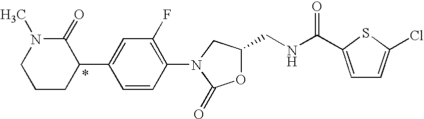

On a preparative scale, the mixture of diastereomers from Example 5 is separated chromatographically into the pure diastereomers. To this end, 432 mg of the compound from Example 5 are dissolved in a mixture of 10 ml of methanol, 10 ml of tert-butyl methyl ether and 5 ml of acetonitrile and chromatographed in ten portions. This gives 156 mg (36% of theory) of the title compound (diastereomer 2) and 182 mg (42% of theory) of diastereomer 1.

Method: column: Daicel Chiralpak IA-H, 5 μm, 250 mm×20 mm; flow rate: 15 ml/min; temperature: 30° C.; UV detection: 220 nm; mobile phase: tert-butyl methyl ether/methanol 1:1.

Retention time: 5.91 min (diastereomer 1), 8.81 min (diastereomer 2)

¹H-NMR (500 MHz, DMSO-d₆, δ/ppm): 8.99 (t, 1H), 7.71 (d, 1H), 7.37 (dd, 1H), 7.21 (d, 1H), 7.13 (dd, 1H), 7.03 (dd, 1H), 4.88-4.83 (m, 1H), 4.10 (t, 1H), 3.77 (dd, 1H), 3.65-3.57 (m, 3H), 3.44-3.39 (m, 1H), 3.33-3.30 (m, 1H, partially obscured by the signal for water), 2.86 (s, 3H), 2.06-2.00 (m, 1H), 1.92-1.75 (m, 3H).

HPLC (method 1): Rₜ=3.92 min.

MS (DCl, NH₃, m/z): 483/485 (³⁵Cl/³⁷Cl) (M+NH₄)⁺.

### Example 8

### 5-Chloro-N-{[(5S)-3-{2-fluoro-4-[3-(hydroxymethyl)-2-oxopiperidin-1-yl]phenyl}-2-oxo-1,3-oxazolidin-5-yl]methyl}thiophene-2-carboxamide (Mixture of diastereomers)

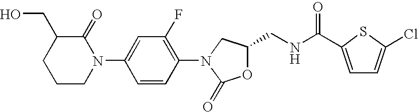

Analogously to the process described under Example 2, 533 mg (0.74 mmol) of the compound from Example 20A give 266 mg (75% of theory) of the title compound.

¹H-NMR (400 MHz, DMSO-d₆, δ/ppm): 8.97 (t, 1H), 7.70 (d, 1H), 7.47 (dd, 1H), 7.31 (dd, 1H), 7.20 (d, 1H), 7.17 (dd, 1H), 4.90-4.83 (m, 1H), 4.63 (t, 1H), 4.11 (dd, 1H), 3.80 (dd, 1H), 3.73-3.56 (m, 6H, partially obscured by the signal for water), 2.51-2.44 (m, 1H, partially obscured by the signal for DMSO), 2.00-1.92 (m, 2H), 1.88-1.77 (m, 2H).

HPLC (method 2): Rₜ=3.80 min.

MS (DCl, NH₃, m/z): 499/501 (³⁵Cl/³⁷Cl) (M+NH₄)⁺.

### Example 9

### 5-Chloro-N-{[(5S)-3-{2-fluoro-4-[3-(hydroxymethyl)-2-oxopiperidin-1-yl]phenyl}-2-oxo-1,3-oxazolidin-5-yl]methyl}thiophene-2-carboxamide (diastereomer 1)

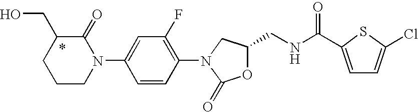

On a preparative scale, the mixture of diastereomers from Example 8 is separated chromatographically into the pure diastereomers. To this end, 223 mg of the compound from Example 8 are dissolved in 20 ml of the solvent and chromatographed in 50 portions. This gives 105 mg (47% of theory) of the title compound (diastereomer 1) and 114 mg (51% of theory) of diastereomer 2.

Method: column: Daicel Chiralpak IA-H, 5 μm, 250 mm×20 mm; flow rate: 15 ml/min; temperature: 30° C.; UV detection: 220 nm; mobile phase: tert-butyl methyl ether/methanol 1:1.

Retention time: 7.14 min (diastereomer 1), 8.05 min (diastereomer 2)

¹H-NMR (400 MHz, DMSO-d₆, δ/ppm): 8.98 (t, 1H), 7.70 (d, 1H), 7.47 (dd, 1H), 7.31 (dd, 1H), 7.20 (d, 1H), 7.17 (dd, 1H), 4.90-4.83 (m, 1H), 4.64 (t, 1H), 4.11 (dd, 1H), 3.79 (dd, 1H), 3.73-3.56 (m, 6H, partially obscured by the signal for water), 2.51-2.44 (m, 1H, partially obscured by the signal for DMSO), 2.00-1.91 (m, 2H), 1.88-1.77 (m, 2H).

HPLC (method 2): Rₜ=3.75 min.

MS (DCl, NH₃, m/z): 499/501 (³⁵Cl/³⁷Cl) (M+NH₄)⁺.

### Example 10

### 5-Chloro-N-{[(5S)-3-{2-fluoro-4-[3-(hydroxymethyl)-2-oxopiperidin-1-yl]phenyl}-2-oxo-1,3-oxazolidin-5-yl]methyl}thiophene-2-carboxamide (diastereomer 2)

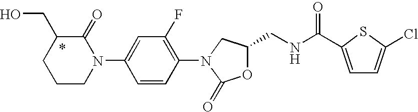

On a preparative scale, the mixture of diastereomers from Example 8 is separated chromatographically into the pure diastereomers. To this end, 223 mg of the compound from Example 8 are dissolved in 20 ml of the solvent and chromatographed in 50 portions. This gives 114 mg (51% of theory) of the title compound (diastereomer 2) and 105 mg (47% of theory) of diastereomer 1.

Method: column: Daicel Chiralpak IA-H, 5 μm, 250 mm×20 mm; flow rate: 15 ml/min; temperature: 30° C.; UV detection: 220 nm; mobile phase: tert-butyl methyl ether/methanol 1:1.

Retention time: 7.14 min (diastereomer 1), 8.05 min (diastereomer 2)

¹H-NMR (400 MHz, DMSO-d₆, δ/ppm): 8.98 (t, 1H), 7.70 (d, 1H), 7.47 (dd, 1H), 7.31 (dd, 1H), 7.20 (d, 1H), 7.16 (dd, 1H), 4.90-4.83 (m, 1H), 4.63 (t, 1H), 4.11 (dd, 1H), 3.80 (dd, 1H), 3.73-3.56 (m, 6H, partially obscured by the signal for water), 2.51-2.44 (m, 1H, partially obscured by the signal for DMSO), 2.00-1.91 (m, 2H), 1.88-1.77 (m, 2H).

HPLC (method 2): Rₜ=3.75 min.

MS (DCl, NH₃, m/z): 499/501 (³⁵Cl/³⁷Cl) (M+NH₄)⁺.

### Example 11

### 5-Chloro-N-({(5S)-3-[2-fluoro-4-(2-oxopiperidin-1-yl)phenyl]-2-oxo-1,3-oxazolidin-5-yl}methyl)thiophene-2-carboxamide

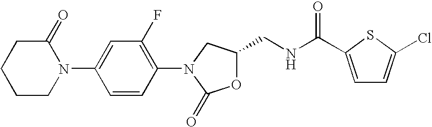

Analogously to the process described under Example 1A, 1.19 g (2.81 mmol) of the product from Example 23A and 911 mg (5.62 mmol) of carbonyldiimidazole give 910 mg (72% of theory) of the title compound. The reaction time is two days.

¹H-NMR (400 MHz, DMSO-d₆, δ/ppm): 8.97 (t, 1H), 7.70 (d, 1H), 7.48 (dd, 1H), 7.32 (dd, 1H), 7.21 (d, 1H), 7.17 (dd, 1H), 4.90-4.83 (m, 1H), 4.11 (t, 1H), 3.80 (dd, 1H), 3.66-3.57 (m, 4H), 2.39 (dd, 2H), 1.89-1.79 (m, 4H).

HPLC (method 1): Rₜ=3.97 min.

MS (DCl, NH₃, m/z): 469/471 (³⁵Cl/³⁷Cl) (M+NH₄)⁺.

### Example 12

### 5-Chloro-N-({(5S)-3-[2-chloro-4-(3-oxomorpholin-4-yl)phenyl]-2-oxo-1,3-oxazolidin-5-yl}methyl)thiophene-2-carboxamide

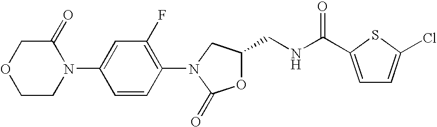

Analogously to the process described under Example 1, 407 mg (0.916 mmol) of the compound from Example 25A and 297 mg (1.83 mmol) of carbonyldiimidazole are converted into 31 mg (7% of theory) of the title compound. Since the product fraction obtained after preparative HPLC was still impure, the product was purified by flash chromatography (silica gel, dichloromethane/methanol 10:1).

¹H-NMR (400 MHz, DMSO-d₆, δ/ppm): 9.00 (t, 1H), 7.73 (d, 1H), 7.69 (d, 1H), 7.54 (d, 1H), 7.47 (dd, 1H), 7.21 (d, 1H), 4.92-4.87 (m, 1H), 4.21 (s, 2H), 4.06 (t, 1H), 3.97 (dd, 2H), 3.78-3.72 (m, 3H), 3.71-3.57 (m, 2H).

HPLC (method 1): Rₜ=4.18 min.

MS (ES+, m/z): 470/472/474 (Cl₂, ³⁵Cl/³⁷Cl) (M+H)⁺.

### Example 13

### 5-Chloro-N-({(5S)-3-[2-fluoro-5-methyl-4-(3-oxomorpholin-4-yl)phenyl]-2-oxo-1,3-oxazolidin-5-yl}methyl)thiophene-2-carboxamide

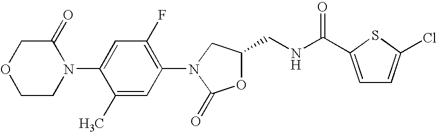

Analogously to the process described under Example 1, 193 mg (0.437 mmol) of the compound from Example 28A and 141 mg (0.873 mmol) of carbonyldiimidazole are converted into 129 mg (63% of theory) of the title compound. The reaction time is 40 hours.

¹H-NMR (400 MHz, DMSO-d₆, δ/ppm): 8.98 (t, 1H), 7.71 (d, 1H), 7.38 (d, 1H), 7.36 (d, 1H), 7.21 (d, 1H), 4.91-4.83 (m, 1H), 4.20 (broad, 2H), 4.12 (t, 1H), 3.97 (dd, 2H), 3.80 (dd, 1H), 3.70 (broad, 1H), 3.68-3.54 (m, 2H), 3.47 (broad, 1H), 2.07 (s, 3H).

HPLC (method 1): Rₜ=3.78 min.

MS (DCl, NH₃, m/z): 468/470 (³⁵Cl/³⁷Cl) (M+H)⁺, 458/487 (M+NH₄)⁺.

### Example 14

### 5-Chloro-N-({(5S)-3-[2-fluoro-5-methyl-4-(2-oxopiperidin-1-yl)phenyl]-2-oxo-1,3-oxazolidin-5-yl}methyl)thiophene-2-carboxamide

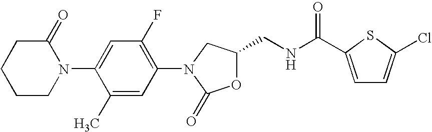

Analogously to the process described under Example 1, 175 mg (0.398 mmol) of the compound from Example 30A and 129 mg (0.796 mmol) of carbonyldiimidazole are converted into 126 mg (64% of theory) of the title compound. The reaction time is 40 hours.

¹H-NMR (400 MHz, DMSO-d₆, δ/ppm): 8.98 (t, 1H), 7.71 (d, 1H), 7.33 (d, 1H), 7.23 (d, 1H), 7.20 (d, 1H), 4.90-4.84 (m, 1H), 4.10 (t, 1H), 3.80 (dd, 1H), 3.67-3.53 (m, 3H), 3.31-3.28 (m, 1H, partially obscured by the signal for water), 2.39-2.31 (m, 2H), 2.02 (s, 3H), 1.90-1.80 (m, 4H).

HPLC (method 1): Rₜ=3.95 min.

MS (DCl, NH₃, m/z): 466/468 (³⁵Cl/³⁷Cl) (M+H)⁺, 483/485 (M+NH₄)⁺.

### Example 15

### 5-Chloro-N-({(5S)-3-[2-fluoro-4-(3-methyl-2-oxotetrahydropyrimidin-1(2H)-yl)phenyl]-2-oxo-1,3-oxazolidin-5-yl}methyl)thiophene-2-carboxamide

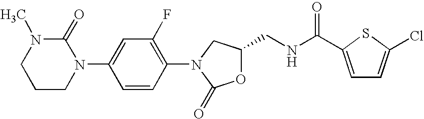

Analogously to the process described under Example 1, 879 mg (1.99 mmol) of the compound from Example 33A and 646 mg (3.99 mmol) of carbonyldiimidazole are converted into 512 mg (55% of theory) of the title compound. The reaction time is 40 hours.

¹H-NMR (400 MHz, DMSO-d₆, δ/ppm): 8.97 (t, 1H), 7.71 (d, 1H), 7.37 (dd, 1H), 7.27 (dd, 1H), 7.21 (d, 1H), 7.12 (dd, 1H), 4.89-4.83 (m, 1H), 4.08 (t, 1H), 3.76 (dd, 1H), 3.65 (dd, 2H), 3.63-3.59 (m, 2H), 3.32 (dd, 2H, partially obscured by the signal for water), 2.87 (s, 3H), 2.05-1.99 (m, 2H).

HPLC (method 1): Rₜ=3.96 min.

MS (ES+, m/z): 467/469 (³⁵Cl/³⁷Cl) (M+H)⁺.

### Example 16

### 5-Chloro-N-{[(5S)-3-{2-fluoro-4-[3-(2-hydroxyethyl)-2-oxotetrahydropyrimidin-1(2H)-yl]phenyl}-2-oxo-1,3-oxazolidin-5-yl]methyl}thiophene-2-carboxamide

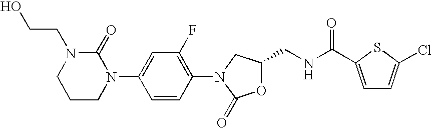

Analogously to the process described under Example 2, 594 mg (0.808 mmol) of the compound from Example 38A give 340 mg (85% of theory) of the title compound.

¹H-NMR (400 MHz, DMSO-d₆, δ/ppm): 8.98 (t, 1H), 7.71 (d, 1H), 7.37 (dd, 1H), 7.29 (dd, 1H), 7.21 (d, 1H), 7.13 (dd, 1H), 4.89-4.82 (m, 1H), 4.67 (t, 1H), 4.09 (t, 1H), 3.76 (dd, 1H), 3.67-3.59 (m, 4H), 3.54-3.50 (m, 2H), 3.43 (dd, 2H), 3.35-3.29 (m, 2H, partially obscured by the signal for water), 2.03-1.98 (m, 2H).

HPLC (method 2): Rₜ=3.77 min.

MS (DCl, NH₃, m/z): 514/516 (³⁵Cl/³⁷Cl) (M+NH₄)⁺.

### Example 17

### 5-Chloro-N-({(5S)-3-[2-fluoro-4-(1-methyl-2-oxo-1,2-dihydropyridin-3-yl)phenyl]-2-oxo-1,3-oxazolidin-5-yl}methyl)thiophene-2-carboxamide

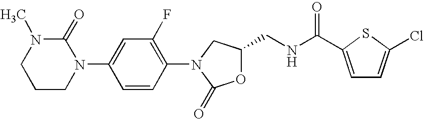

Analogously to the process described under Example 1, 350 mg (0.803 mmol) of the compound from Example 42A and 260 mg (1.61 mmol) of carbonyldiimidazole are converted into 88 mg (24% of theory) of the title compound.

¹H-NMR (400 MHz, DMSO-d₆, δ/ppm): 9.00 (t, 1H), 7.81-7.71 (m, 4H), 7.59 (dd, 1H), 7.50 (dd, 1H), 7.21 (d, 1H), 6.35 (dd, 1H), 4.91-4.85 (m, 1H), 4.14 (t, 1H), 3.83 (dd, 1H), 3.69-3.57 (m, 2H), 3.52 (s, 3H).

HPLC (method 1): Rₜ=3.97 min.

MS (ES+, m/z): 462/464 (³⁵Cl/³⁷Cl) (M+H)⁺.

### Example 18

### 5-Chloro-N-({(5S)-3-[2-fluoro-4-(2-hydroxypyridin-3-yl)phenyl]-2-oxo-1,3-oxazolidin-5-yl}methyl)thiophene-2-carboxamide

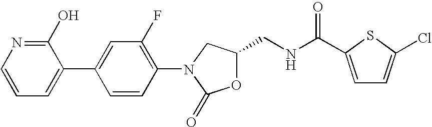

Analogously to the process described under Example 1, 208 mg (0.493 mmol) of the compound from Example 45A and 160 mg (0.986 mmol) of carbonyldiimidazole are converted into 121 mg (55% of theory) of the title compound.

¹H-NMR (500 MHz, DMSO-d₆, δ/ppm): 11.92 (s, broad, 1H), 9.00 (t, 1H), 7.80-7.76 (m, 2H), 7.70 (d, 1H), 7.61 (dd, 1H), 7.49 (dd, 1H), 7.43 (dd, 1H), 7.21 (d, 1H), 6.31 (dd, 1H), 4.90-4.86 (m, 1H), 4.13 (t, 1H), 3.82 (dd, 1H), 3.67-3.58 (m, 2H).

HPLC (method 1): Rₜ=3.84 min.

MS (ES+, m/z): 448/450 (³⁵Cl/³⁷Cl) (M+H)⁺.

### Example 19

### 5-Chloro-N-({(5S)-3-[2-fluoro-4-(2-oxopyridin-1(2H)-yl)phenyl]-2-oxo-1,3-oxazolidin-5-yl}methyl)thiophene-2-carboxamide

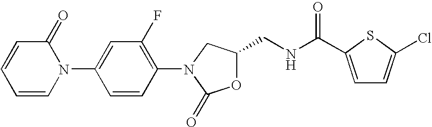

Analogously to the process described under Example 1, 750 mg (1.78 mmol) of the compound from Example 47A and 577 mg (3.56 mmol) of carbonyldiimidazole are converted into 388 mg (49% of theory) of the title compound. On addition of water to the reaction mixture once the reaction has ended, a first fraction of the product (130 mg) precipitates as a solid. A further fraction of the product (258 mg) is obtained after preparative HPLC (method 11) of the crude product of aqueous work-up.

¹H-NMR (400 MHz, DMSO-d₆, δ/ppm): 9.00 (t, 1H), 7.71 (d, 1H), 7.69-7.61 (m, 2H), 7.54-7.50 (m, 2H), 7.32 (dd, 1H), 7.21 (d, 1H), 6.50 (d, 1H), 6.33 (dd, 1H), 4.93-4.88 (m, 1H), 4.18 (t, 1H), 3.87 (dd, 1H), 3.69-3.58 (m, 2H).

HPLC (method 1): Rₜ=3.84 min.

MS (DCl, NH₃, m/z): 465/467 (³⁵Cl/³⁷Cl) (M+NH₄)⁺.

### Example 20

### 5-Chloro-N-{[(5S)-3-{2-fluoro-4-[3-(2-hydroxyethoxy)-2-oxopyridin-1(2H)-yl]phenyl}-2-oxo-1,3-oxazolidin-5-yl]methyl}thiophene-2-carboxamide

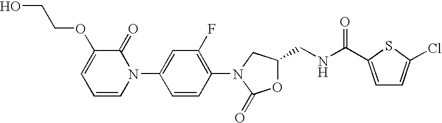

Analogously to the process described under Example 2, 60 mg (0.096 mmol) of the compound from Example 51A give 34 mg (69% of theory) of the title compound.

¹H-NMR (400 MHz, DMSO-d₆, δ/ppm): 8.99 (t, 1H), 7.71 (d, 1H), 7.63 (dd, 1H), 7.52 (dd, 1H), 7.31 (dd, 1H), 7.23 (dd, 1H), 7.21 (d, 1H), 6.92 (dd, 1H), 6.24 (dd, 1H), 4.93-4.88 (m, 1H), 4.90 (t, 1H), 4.18 (t, 1H), 3.94 (t, 2H), 3.87 (dd, 1H), 3.72 (quart, 2H), 3.65-3.61 (m, 2H).

LC/MS (method 1): Rₜ=3.75 min.

MS (ES+, m/z): 508/510 (³⁵Cl/³⁷Cl) (M+H)⁺.

### Example 21

### 5-Chloro-N-{[(5S)-3-{2-fluoro-4-[3-(2-hydroxyethyl)-2-oxopyridin-1(2H)-yl]phenyl}-2-oxo-1,3-oxazolidin-5-yl]methyl}thiophene-2-carboxamide

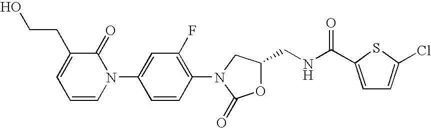

1.9 ml of water, 92 μl of a 2.5% strength solution of osmium tetraoxide in tert-butanol and 235 mg (1.10 mmol) of sodium periodate are added to a solution of 179 mg (0.367 mmol) of the product from Example 56A in 1.9 ml of THF. The reaction mixture is stirred at room temperature for 15 hours. The mixture is then diluted with water and extracted with dichloromethane. After drying over anhydrous magnesium sulphate, the organic extract is filtered and freed from the solvent on a rotary evaporator. The residue obtained is dissolved again in 2 ml of THF, and 2 ml of water and 14 mg (0.367 mmol) of sodium borohydride are added. The mixture is stirred at room temperature for one hour. The mixture is then once more—as described above—diluted with water and extracted with dichloromethane. The crude product obtained is initially pre-purified by preparative HPLC (method 11). This gives 22 mg of the title compound as a mixture with the compound from Example 22 (see below). The two substances are separated from one another by preparative HPLC. To this end, the 22 mg are dissolved in 4 ml of acetonitrile/water 3:1 and chromatographed in 4 portions.

Chromathographic method: column: Kromasil 100C18, 5 μm, 250 mm×20 mm; flow rate: 25 ml/min; temperature: 40° C.; UV detection: 210 nm; mobile phase: water/acetonitrile 3:1.

This gives 3.2 mg (1.8% of theory) of the title compound and 10.2 mg (5.8% of theory) of the product from Example 22 (see below).

¹H-NMR (400 MHz, DMSO-d₆, δ/ppm): 8.99 (t, 1H), 7.70 (d, 1H), 7.62 (dd, 1H), 7.54-7.49 (m, 2H), 7.38 (dd, 1H), 7.30 (dd, 1H), 7.20 (d, 1H), 6.27 (dd, 1H), 4.93-4.87 (m, 1H), 4.58 (t, 1H), 4.17 (t, 1H), 3.85 (dd, 1H), 3.70-3.49 (m, 2H), 3.48 (quart, 2H), 2.60 (t, 2H).

LC/MS (method 4): Rₜ=1.86 min.

MS (ES+, m/z): 492/494 (³⁵Cl/³⁷Cl) (M+H)⁺.

### Example 22

### 5-Chloro-N-{[(5S)-3-{2-fluoro-4-[3-(hydroxymethyl)-2-oxopyridin-1(2H)-yl]phenyl}-2-oxo-1,3-oxazolidin-5-yl]methyl}thiophene-2-carboxamide

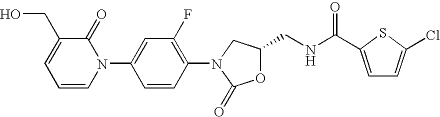

The preparation of the title compound is described in Example 21.

¹H-NMR (400 MHz, DMSO-d₆, δ/ppm): 8.99 (t, 1H), 7.71 (d, 1H), 7.63 (dd, 1H), 7.58 (dd, 1H), 7.54-7.50 (m, 2H), 7.31 (dd, 1H), 7.21 (d, 1H), 6.38 (dd, 1H), 5.14 (t, 1H), 4.93-4.88 (m, 1H), 4.32 (d, 2H), 4.18 (t, 1H), 3.86 (dd, 1H), 3.69-3.59 (m, 2H).

LC/MS (method 4): Rₜ=1.83 min.

MS (ES+, m/z): 478/480 (³⁵Cl/³⁷Cl) (M+H)⁺.

### Example 23

### 5-Chloro-N-({(5S)-3-[2-chloro-4-(3-methyl-2-oxopyridin-1(2H)-yl)phenyl]-2-oxo-1,3-oxazolidin-5-yl}methyl)thiophene-2-carboxamide

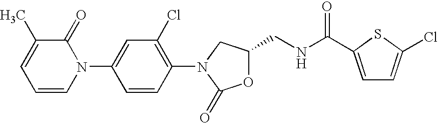

328 mg (1.47 mmol) of magnesium perchlorate are added to a solution of 230 mg (0.98 mmol) of the compound from Example 58A and 235 mg (1.08 mmol) of the compound from Example 4A in 5 ml of acetonitrile. The reaction mixture is stirred at room temperature for 16 hours. 397 mg (2.45 mmol) of carbonyldiimidazole and 12 mg (0.10 mmol) of 4-(dimethylamino)pyridine are then added, and stirring is continued at 60° C. After 20 hours, the reaction mixture is concentrated on a rotary evaporator and the product is isolated by preparative HPLC (method 11). This gives 106 mg (20% of theory) of the title compound.

¹H-NMR (400 MHz, DMSO-d₆, δ/ppm): 9.02 (t, 1H), 7.73 (d, 1H), 7.71 (d, 1H), 7.65 (d, 1H), 7.55 (dd, 1H), 7.48 (dd, 1H), 7.41 (dd, 1H), 7.21 (d, 1H), 6.26 (dd, 1H), 4.95-4.89 (m, 1H), 4.10 (t, 1H), 3.80 (dd, 1H), 3.73-3.58 (m, 2H).

HPLC (method 2): Rₜ=4.17 min.

MS (DCl, NH₃, m/z): 495/497/499 (Cl₂, ³⁵Cl/³⁷Cl) (M+NH₄)⁺.

### B. EVALUATION OF THE PHARMACOLOGICAL ACTIVITY

The compounds according to the invention act in particular as inhibitors of blood coagulation factor Xa and do not, or only at significantly higher concentrations, inhibit other serine proteases, such as plasmin or trypsin.

The advantageous pharmacological properties of the compounds according to the invention can be determined by the following methods:

### a) Test Descriptions (In Vitro)

### a.1) Determination of the Factor Xa Inhibition

### a.1.1) Chromogenic Assay:

The enzymatic activity of human factor Xa (FXa) is measured using the conversion of a chromogenic substrate specific for FXa. Factor Xa cleaves p-nitroaniline from the chromogenic substrate. The determinations are carried out in microtitre plates as follows:

The test substances, in various concentrations, are dissolved in DMSO and incubated for 10 minutes at 25° C. with human FXa (0.5 nmol/l dissolved in 50 mmol/l of Tris buffer [C,C,C-tris(hydroxymethyl)aminomethane], 150 mmol/l of NaCl, 0.1% BSA [bovine serum albumin], pH=8.3). Pure DMSO is used as control. The chromogenic substrate (150 μmol/l of Pefachrome® FXa from Pentapharm) is then added. After an incubation time of 20 minutes at 25° C., the extinction at 405 nm is determined. The extinctions of the test mixtures containing the test substance are compared with control mixtures without test substance, and the IC₅₀ values are calculated from these data.

### a.1.2) Fluorogenic Assay:

The enzymatic activity of human factor Xa (FXa) is measured using the conversion of a fluorogenic substrate specific for FXa. FXa cleaves aminomethylcoumarin, whose fluorescence is measured, from the peptidic substrate. The determinations are carried out in microtitre plates.

Substances to be tested, in various concentrations, are dissolved in dimethyl sulphoxide and incubated for 15 min at 22° C. with human FXa (1.3 nmol/l dissolved in 50 mmol/l of Tris buffer [C,C,C-tris(hydroxymethyl)aminomethane], 100 mmol/l NaCl, 0.1% BSA [bovine serum albumin], pH 7.4). The fluorogenic substrate (5 μmol/l of Boc-Ile-Glu-Gly-Arg-AMC from Bachem) is then added. After an incubation time of 30 min, the sample is excited at a wavelength of 360 nm, and the emission at 460 nm is measured. The measured emissions of the test batches with test substance are compared to the control batches without test substance (only dimethyl sulphoxide instead of test substance in dimethyl sulphoxide), and IC₅₀ values are calculated from the concentration/activity relationships.

Representative activity data from this test are listed in Table 1 below:

TABLE 1

| Example | |
|---|---|
| No. | IC₅₀ [nM] |
| 1 | 0.9 |
| 11 | 2.2 |
| 12 | 1.8 |
| 22 | 0.9 |

### a.2) Determination of the Selectivity

### a.2.1) Chromogenic Assay:

To demonstrate the selective FXa inhibition, the test substances are examined for their inhibition of other human serine proteases, such as thrombin, trypsin and plasmin. To determine the enzymatic activity of thrombin (75 mU/ml), trypsin (500 mU/ml) and plasmin (3.2 nmol/l), these enzymes are dissolved in Tris buffer (100 mmol/l, 20 mmol/l of CaCl₂, pH=8.0) and incubated with test substance or solvent for 10 minutes. The enzymatic reaction is then started by addition of the appropriate specific chromogenic substrates (Chromozym Thrombin®, Chromozym Trypsin® and Chromozym Plasmin®; from Roche Diagnostics), and after 20 minutes the extinction is determined at 405 nm. All determinations are carried out at 37° C. The extinctions of the test batches with test substance are compared to the control samples without test substance, and the IC₅₀ values are calculated from these data.

### a.2.2) Fluorogenic Assay:

To demonstrate the selectivity of the substances with respect to factor Xa inhibition, the test substances are examined for their inhibition of other human serine proteases, such as thrombin, trypsin and plasmin. To determine the enzymatic activity of thrombin (0.06 nmol/l from Kordia), trypsin (83 mU/ml from Sigma) and plasmin (0.1 μg/ml from Kordia), these enzymes are dissolved (50 mmol/l of Tris buffer [C,C,C-tris(hydroxymethyl)aminomethane], 100 mmol/l of NaCl, 0.1% BSA [bovine serum albumin], 5 mmol/l of calcium chloride, pH 7.4) and incubated for 15 min with various concentrations of test substance in dimethyl sulphoxide and also with dimethyl sulphoxide without test substance. The enzymatic reaction is then started by addition of the appropriate substrates (5 μmol/l of Boc-Asp(OBzl)-Pro-Arg-AMC from Bachem for thrombin, 5 μmol/l of Boc-Ile-Glu-Gly-Arg-AMC from Bachem for trypsin and 50 μmol/l of MeOSuc-Ala-Phe-Lys-AMC from Bachem for plasmin). After an incubation time of 30 min at 22° C., the fluorescence is measured (excitation: 360 nm, emission: 460 nm). The measured emissions of the test batches with test substance are compared to the control batches without test substance (only dimethyl sulphoxide instead of test substance in dimethyl sulphoxide), and IC₅₀ values are calculated from the concentration/activity relationships.

### a.3) Determination of the Anticoagulatory Activity

### a.3.1) Prothrombin Time (PT):

The anticoagulatory activity of the test substances is determined in vitro in human and rabbit plasma. To this end, blood is drawn off in a mixing ratio of sodium citrate/blood of 1:9 using a 0.11 molar sodium citrate solution as receiver. Immediately after the blood has been drawn off, it is mixed thoroughly and centrifuged at about 2500 g for 10 minutes. The supernatant is pipetted off. The prothrombin time (PT, synonyms: thromboplastin time, quick test) is determined in the presence of varying concentrations of test substance or the corresponding solvent using a commercial test kit (Hemoliance® RecombiPlastin, from Instrumentation Laboratory). The test compounds are incubated with the plasma at 37° C. for 3 minutes. Coagulation is then started by addition of thromboplastin, and the time when coagulation occurs is determined. The concentration of test substance which effects a doubling of the prothrombin time is determined.

### a.3.2) Thrombin Generation Assay (Thrombogram)

In the thrombin generation assay according to Hemker, the activity of thrombin in coagulating plasma is determined by measuring the fluorescent cleavage products of the substrate I-1140 (Z-Gly-Gly-Arg-AMC, Bachem). The reactions are carried out in 20 mM Hepes, 60 mg/ml of BSA, 102 mM CaCl₂, pH 7.5 at 37° C. The reactions are carried out in Immulon 2HB clear U-bottom 96-well plates (Thermo Electron) in a total volume of 100 μl. To start the reaction in platelet-poor plasma (PPP) or platelet-rich plasma (PRP), reagents from Thrombinoscope are used (PPP reagent: 30 pM recombinant tissue factor, 24 μM phospholipids in HEPES; PRP reagent: 3 pM recombinant tissue factor). Also required is a calibrator whose amidolytic activity is needed for calculating the thrombin activity in a sample containing an unknown amount of thrombin. The calibrator also allows the data to be corrected for donor variability (different coloration of the plasma), variability by the measuring instrument, the inner filter effect and the substrate consumption. The measurement is carried out using a fluorometer (Fluoroskan Ascent) from Thermo Electron fitted with a 390/460 nM filter pair and a dispenser. Practice of the test: the lyophilisates are dissolved (PPP reagent, PRP reagent, calibrator), the MTPs are incubated at 37° C. for 5 min, FluCa is prepared (70 μl of I-1140+2800 μl of Fluo buffer (20 mM HEPES, 102 mM CaCl₂, 60 mg/ml of BSA, pH 7.5) per plate), the program is started, the dispenser is flushed and the system is filled with FluoCa, 20 μl of FluoCa per well are added and thrombin generation is measured every 20 s, (or in the case of animal plasma every 10 s) over 120 min. The thrombogram is calculated and represented graphically using the thrombinoscope software. The following parameters are stated: lag time (time until the generation of thrombin starts), ttPeak (time to peak, time until the maximum is reached), peak (maximum thrombin concentration), ETP (endogenous thrombin potential, the area under the curve) and start tail (the point in time when the thrombin concentration goes back to 0).

### a.4) Specific Diagnosis of Impaired Coagulation and Organ Function in Endotoxaemic Mice and Rats

### a.4.1) Thrombin/Antithrombin Complexes

Thrombin/antithrombin complexes (hereinbelow referred to as “TAT”) are a measure for the thrombin formed endogenously by coagulation activation. TAT are determined using an ELISA assay (Enzygnost TAT micro, Dade-Behring). Plasma is obtained from citrated blood by centrifugation. 50 μl of TAT sample buffer are added to 50 μl of plasma, and the sample is shaken briefly and incubated at room temperature for 15 min. The samples are filtered off with suction, and the well is washed 3 times with wash buffer (300 μl/well). Between the washing stages, the liquid is removed by tapping the plate. Conjugate solution (100 μl) is added, and the plate is incubated at room temperature for 15 min. The samples are sucked off, and the well is washed 3 times with wash buffer (300 μl/well). Chromogenic substrate (100 μl/well) is then added, the plate is incubated in the dark at room temperature for 30 min, stop solution is added (100 μl/well) and the colour development is measured at 492 nm (Saphire plate reader).

### a.4.2) Parameters for Organ Function

Various parameters are determined which allow conclusions to be drawn with respect to a restriction of the function of various internal organs by administration of LPS and which allow the therapeutic effect of test substances to be estimated. Citrated blood or, if appropriate, lithium/heparin blood is centrifuged, and the parameters are determined from the plasma. Typically, the following parameters are determined: creatinin, urea, aspartate aminotransferase (AST), alanine aminotransferase (ALT), total bilirubin, lactate dehydrogenase (LDH), total protein, total albumin and fibrinogen. The values give indications concerning the function of the kidneys, the liver, the cardiovascular system and the blood vessels.

### a.4.3) Parameters for Inflammation

The extent of the inflammatory reaction triggered by endotoxin can be detected by the increase of inflammation mediators, for example interleukins (1, 6, 8 and 10), tumour necrosis factor alpha or monocyte chemoattractant protein-1 in the plasma. To this end, ELISAs or the luminex system may be used.

### b) Determination of the Antithrombotic Activity (In Vivo)

### b.1) Arteriovenous Shunt Model (Rabbit)

Fasting rabbits (strain: Esd: NZW) are anaesthetized by intramuscular administration of Rompun/Ketavet solution (5 mg/kg and 40 mg/kg, respectively). Thrombus formation is initiated in arteriovenous shunt in accordance with the method described by C. N. Berry et al. [Semin. Thromb. Hemost. 1996, 22, 233-241]. To this end, the left jugular vein and the right carotid artery are exposed. The two vessels are connected by an extracorporeal shunt using a vein catheter of a length of 10 cm. In the middle, this catheter is attached to a further polyethylene tube (PE 160, Becton Dickenson) of a length of 4 cm which contains a roughened nylon thread which has been arranged to form a loop, to form a thrombogenic surface. The extracorporeal circulation is maintained for 15 minutes. The shunt is then removed and the nylon thread with the thrombus is weighed immediately. The weight of the nylon thread on its own was determined before the experiment was started. Before extracorporeal circulation is set up, the test substances are administered either intravenously via an ear vein or orally using a pharyngeal tube.

### b.2) Iron(III) Chloride Model (Rat)

Fasting rats are anaesthetized by intraperitoneal administration of thiobarbital-sodium (180 mg/kg). Arterial thrombus formation is triggered at the carotid artery similarly to the method described by Kurz et al. [Thromb Res. 1990 Nov. 15; 60(4):269-80]. To this end, the right carotid artery is exposed, and a flow sensor is fixed at the vessel (perivascular probe). A filter paper is drenched with 25% strength iron(III) chloride solution and pushed under the carotid artery; in some protocol versions, the filter paper is removed again after a defined period of time (for example after 5 minutes). Before extracorporeal circulation is set up, the test substances are administered either intravenously via an ear vein or orally using a pharyngeal tube. The following parameters are stated: the point in time when the flow starts to be reduced (start of thrombus formation); speed of flow reduction (speed of thrombus formation); occurrence of complete occlusion and interval until complete occlusion.

### b.3) Venous Stasis Model (Rat)

The antithrombotic activity of the substances is examined in an established model for venous thrombosis (method see also Ref. 1-3) in rats. Venous thrombae are generated using a combination of circulatory arrest and thromboplastin injection. Male rats (HSD CPB:WU; Harlan Winkelmann) having a weight of 220 g-260 g are fasted overnight. Water is available ad libitum. Prior to the start of the test, the animals are anaesthetized by intraperitoneal administration of a xylazine/ketamine mixture (5 ml/kg) (Rompun Bayer 12 mg/kg, Ketavet Pharmacia & Upjohn GmbH, 50 mg/kg). The left jugular vein and the abdominal vena cava are exposed. A catheter is pushed into the jugular vein. Proximally and distally at a distance of 8-10 mm, a loop is placed around the vena cava so that this section of the vein can later be tied off. To start the formation of the thrombus, thromboplastin (Neoplastin Plus, Diagnostica Stago, Roche) is injected over a period of 15 seconds into the jugular vein (0.5 mg/kg in 1 ml/kg). After a further 15 seconds, the vena cava is tied off, initially proximally and then, after 30 seconds, distally. The ligated segment of the vein is excised 15 minutes after the thromboplastin injection. The thrombus is exposed and weighed immediately. The inhibitors to be examined (1 ml/kg) are administered intravenously to the animals prior to the preparation.

### b.4) Haemorrhage Model (Rat)

Fasting male rats (strain: HSD CPB:WU) having a weight of 300-350 g are anaesthetized using Inactin (150-180 mg/kg). To determine the bleeding time, immediately after opening of the shunt circulation, the tip of the tail of the rats is docked by 3 mm using a razor blade. The tail is then placed into physiological saline solution kept at a temperature of 37° C., and the bleeding from the cut is observed over a period of 15 min. What is determined are the time until bleeding ceases for at least 30 seconds (initial bleeding time), total bleeding time over a period of 15 minutes (cumulative bleeding time) and the quantitative blood loss via photometric determination of the collected haemoglobin.

Before the extracorporeal circulation is set up and the tip of the tail is docked, the test substances are administered to the animals while awake either intravenously via the contralateral jugular vein as a single bole or as a bole with subsequent continuous infusion or orally using a pharyngeal tube.

### b.5) Pharmacokinetic/Pharmacodynamic Model (Rat)

Fasting rats are anaesthetized by intraperitoneal administration of thiobarbital-sodium (Inactin) (180 mg/kg). A catheter (PE 190) is pushed into the abdominal aorta, and blood is withdrawn to determine the substance plasma concentration and ex vivo blood coagulation (FXa, PT, aPTT, Thrombin Generation Assay, etc.). The substances are administered orally at various points in time prior to blood withdrawal. The substances are administered in dosages of 1 and 5 mg/kg p.o. and blood is in each case withdrawn at a later point in time (6 and 10 hours after substance administration).

### c) Solubility Assay

### Reagents Required:

PBS buffer pH 7.4: 90.00 g of NaCl p.a. (for example Merck Art. No. 1.06404.1000), 13.61 g of KH₂PO₄ p.a. (for example Merck Art. No. 1.04873.1000) and 83.35 g of 1N NaOH (for example Bernd Kraft GmbH Art. No. 01030.4000) are weighed into a 1 l measuring flask, the flask is filled with water and the mixture is stirred for about 1 hour.
Acetate buffer pH 4.6: 5.4 g of sodium acetate×3H₂O p.a. (for example Merck Art. No. 1.06267.0500) are weighed into a 100 ml measuring flask and dissolved in 50 ml of water, 2.4 g of glacial acetic acid are added, the mixture is made up to 100 ml with water, the pH is checked and, if required, adjusted to pH 4.6.
Dimethyl sulphoxide (for example Baker Art. No. 7157.2500)
Distilled water

### Preparation of the Calibration Solutions:

Preparation of the stock solution of calibration solutions: About 0.5 mg of the active compound are weighed accurately into a 2 ml Eppendorf Safe-Lock tube (Eppendorf Art. No. 0030 120.094), DMSO is added to a concentration of 600 μg/ml (for example 0.5 mg of active compound+833 μl of DMSO) and the mixture is vortexed until everything has gone into solution.

Calibration solution 1 (20 μg/ml): 1000 μl of DMSO are added to 34.4 μl of the stock solution, and the mixture is homogenized.

Calibration solution 2 (2.5 μg/ml): 700 μl of DMSO are added to 100 μl of calibration solution 1, and the mixture is homogenized.

### Preparation of the Sample Solutions:

Sample solution for solubilities of up to 10 g/l in PBS buffer pH 7.4: About 5 mg of the active compound are weighed accurately into a 2 ml Eppendorf Safe-Lock tube (Eppendorf Art. No. 0030 120.094), and PBS buffer pH 7.4 is added to a concentration of 5 g/l (for example 5 mg of active compound+500 μl of PBS buffer pH 7.4).

Sample solution for solubilities of up to 10 g/l in acetate buffer pH 4.6: About 5 mg of the active compound are weighed accurately into a 2 ml Eppendorf Safe-Lock tube (Eppendorf Art. No. 0030 120.094), and acetate buffer pH 4.6 is added to a concentration of 5 g/l (for example 5 mg of active compound+500 μl of acetate buffer pH 4.6).

Sample solution for solubilities of up to 10 g/l in water: About 5 mg of the active compound are weighed accurately into a 2 ml Eppendorf Safe-Lock tube (Eppendorf Art. No. 0030 120.094), and water is added to a concentration of 5 g/l (for example 5 mg of active compound+500 μl of water).

### Practice:

The sample solutions prepared in this manner are shaken at 1400 rpm in a temperature-adjustable shaker (for example Eppendorf Thermomixer comfort Art. No. 5355 000.011 with interchangeable block Art. No. 5362.000.019) at 20° C. for 24 hours. In each case 180 μl are taken from these solutions and transferred into Beckman Polyallomer centrifuge tubes (Art. No. 343621). These solutions are centrifuged at about 223 000 *g for 1 hour (for example Beckman Optima L-90K ultracentrifuge with type 42.2 Ti rotor at 42 000 rpm). From each of the sample solutions, 100 μl of the supernatant are removed and diluted 1:5, 1:100 and 1:1000 with the respective solvent used (water, PBS buffer 7.4 or acetate buffer pH 4.6). From each dilution, a sample is transferred into a vessel suitable for HPLC analysis.

### Analysis:

The samples are analyzed by RP-HPLC. Quantification is carried out using a two-point calibration curve of the test compound in DMSO. The solubility is expressed in mg/l.

### Analysis Sequence:

Calibration solution 2.5 mg/ml
Calibration solution 20 μg/ml
Sample solution 1:5
Sample solution 1:100
Sample solution 1:1000

### HPLC Method for Acids:

Agilent 1100 with DAD (G1315A), quat. pump (G1311A), autosampler CTC HTS PAL, degasser (G1322A) and column thermostat (G1316A); column: Phenomenex Gemini C18, 50×2 mm, 5μ; temperature: 40° C.; mobile phase A: water/phosphoric acid pH 2; mobile phase B: acetonitrile; flow rate: 0.7 ml/min; gradient: 0-0.5 min 85% A, 15% B; ramp: 0.5-3 min 10% A, 90% B; 3-3.5 min 10% A, 90% B; ramp: 3.5-4 min 85% A, 15% B; 4-5 min 85% A, 15% B.

### HPLC Method for Bases:

Agilent 1100 with DAD (G1315A), quat. pump (G1311A), autosampler CTC HTS PAL, degasser (G1322A) and column thermostat (G1316A); column: VDSoptilab Kromasil 100C18, 60×2.1 mm, 3.5 p; temperature: 30° C.; mobile phase A: water+5 ml perchloric acid/l; mobile phase B: acetonitrile; flow rate: 0.75 ml/min; gradient: 0-0.5 min 98% A, 2% B; ramp: 0.5-4.5 min 10% A, 90% B; 4.5-6 min 10% A, 90% B; ramp: 6.5-6.7 min 98% A, 2% B; 6.7-7.5 min 98% A, 2% B.

### d) Determination of Pharmacokinetics (In Vivo)

To determine the in vivo pharmacokinetics, the test substances are dissolved in various formulating compositions (for example plasma, ethanol, DMSO, PEG400, etc.) or mixtures of these solubilizers and administered intravenously or perorally in male or female Wistar rats. Intravenous administration is carried out either as a bolus injection or as an infusion. The doses administered are in the range from 0.1 to 5 mg/kg. Blood samples are taken by means of a catheter or as sacrifice plasma at various times over a period of up to 26 h. Quantitative determination of the substances in the test samples takes place in plasma using calibration samples adjusted in plasma. Proteins present in the plasma are removed by precipitation with acetonitrile. The samples are then fractionated by HPLC using reversed-phase columns in a 2300 HTLC system (Cohesive Technologies, Franklin, Mass., USA). The HPLC system is coupled via a turbo ion spray interface to an API 3000 Triple Quadropole mass spectrometer (Applied Biosystems, Darmstadt, Germany). The plasma concentration time course is analyzed using a validated kinetic analysis program.

### e) Determination of the Endotoxinaemia Activity (In Vivo)

The examination is carried out using rats or mice. In the mouse model (NMRI, male), LPS (Escherichia coli serotype 055:B5, Sigma-Aldrich) is injected 50 mg/kg intraperitoneally. The test substances are administered up to one hour prior to the LPS injection either intravenously via the tail vein, subcutaneously, intraperitoneally or orally using a pharyngeal tube. Four hours after the LPS administration, the animal is anaesthetized (Ketavet/Rompun) and the abdomen is opened by surgery. Sodium citrate solution (3.2% w/v) (formula: body weight in g/13 times 100 μl) is injected into the lower vena carva, and a blood sample (about 1 ml) is taken after 30 sec. Various parameters, for example cellular blood components (in particular erythrocytes, leukocytes and platelets), lactate concentration, coagulation activation (TAT) or parameters of organ dysfunction or organ failure and mortality are determined from the blood.

### f) Description of the Method Used for DIC Tests on Rats

LPS (E. coli 055 B5, manufactured by Sigma, dissolved in PBS) is administered to male Wistar rats at a dosage of 250 μg/kg intravenously into the tail vein (administration volume 2 ml/kg). The test substance is dissolved in PEG 400/H₂O 60%/40% and administered orally (administration volume 5 ml/kg) 30 minutes prior to the LPS injection. 1, 5 or 4 hours after the LPS injection, the animals are exsanguinated by puncture of the heart in terminal anaesthesia (Trapanal® 100 mg/kg i.p.), and citrate plasma is obtained for the determination of fibrinogen, PT, TAT and platelet number. Optionally, serum is obtained for the determination of liver enzymes, kidney function parameters and cytokines. TNFα and IL-6 are determined using commercially available ELISAs (R&D Systems).

It is also possible to measure direct parameters of organ function, for example left- and right-ventricular pressures, arterial pressures, urine excretion, kidney perfusion and blood gases and acid/base state.

### C. EXEMPLARY EMBODIMENTS OF PHARMACEUTICAL COMPOSITIONS

The compounds according to the invention can be converted into pharmaceutical preparations in the following ways:

### Tablet:

### Composition:

100 mg of the compound according to the invention, 50 mg of lactose (monohydrate), 50 mg of corn starch (native), 10 mg of polyvinylpyrrolidone (PVP 25) (from BASF, Ludwigshafen, Germany) and 2 mg of magnesium stearate.

Tablet weight 212 mg. Diameter 8 mm, radius of curvature 12 mm.

### Preparation:

The mixture of the compound according to the invention, lactose and starch is granulated with a 5% strength solution (m/m) of PVP in water. The granules are dried and then mixed with the magnesium stearate for 5 minutes. This mixture is compressed using a conventional tablet press (see above for format of the tablet). As guideline, a compressive force of 15 kN is used for the compression.

### Oral Suspension:

### Composition:

1000 mg of the compound according to the invention, 1000 mg of ethanol (96%), 400 mg of Rhodigel® (xanthan gum from FMC, Pennsylvania, USA) and 99 g of water.

10 ml of oral suspension are equivalent to a single dose of 100 mg of the compound according to the invention.

### Preparation:

The Rhodigel is suspended in ethanol, and the compound according to the invention is added to the suspension. The water is added while stirring. The mixture is stirred for about 6 h until the swelling of the Rhodigel is complete.

### Oral Solution:

### Composition:

500 mg of the compound according to the invention, 2.5 g of polysorbate and 97 g of polyethylene glycol 400. 20 g of oral solution are equivalent to a single dose of 100 mg of the compound according to the invention.

### Production:

The compound according to the invention is suspended in the mixture of polyethylene glycol and polysorbate while stirring. Stirring is continued until the compound according to the invention is completely dissolved.

i.v. Solution:

The compound according to the invention is dissolved at a concentration below saturation solubility in a physiologically acceptable solvent (for example isotonic sodium chloride solution, glucose solution 5% and/or PEG 400 solution 30%). The solution is sterilized by filtration and filled into sterile and pyrogen-free injection containers.

1. Compound of the formula 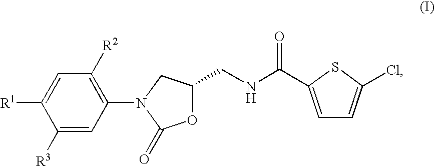
in which
R¹ represents a group of the formula 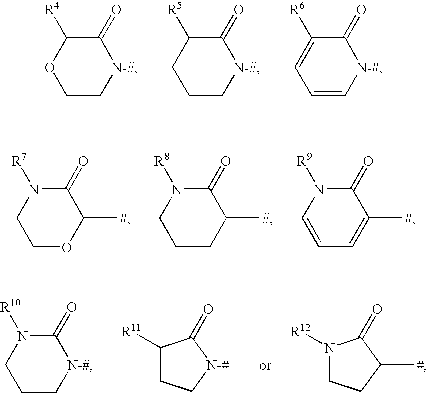
where
# is the point of attachment to the phenyl ring,
R⁴ represents hydrogen or C₁-C₃-alkyl,
where alkyl may be substituted by a substituent, where the substituent is selected from the group consisting of hydroxyl, C₁-C₃-alkoxy and C₃-C₆-cycloalkyloxy,
R⁵ represents hydrogen, hydroxyl, C₁-C₃-alkyl, C₁-C₃-alkoxy or C₃-C₆-cycloalkyloxy,
where alkyl and alkoxy may be substituted by a substituent, where the substituent is selected from the group consisting of hydroxyl, C₁-C₃-alkoxy and C₃-C₆-cycloalkyloxy,
R⁶ represents hydrogen, hydroxyl, C₁-C₃-alkyl, C₁-C₃-alkoxy or C₃-C₆-cycloalkyloxy,
where alkyl and alkoxy may be substituted by a substituent, where the substituent is selected from the group consisting of hydroxyl, C₁-C₃-alkoxy and C₃-C₆-cycloalkyloxy,
R⁷ represents hydrogen, C₁-C₃-alkyl or C₃-C₆-cycloalkyl,
where C₂-C₃-alkyl may be substituted by a substituent, where the substituent is selected from the group consisting of hydroxyl, C₁-C₃-alkoxy and C₃-C₆-cycloalkyloxy,
R⁸ represents hydrogen, C₁-C₃-alkyl or C₃-C₆-cycloalkyl,
where C₂-C₃-alkyl may be substituted by a substituent, where the substituent is selected from the group consisting of hydroxyl, C₁-C₃-alkoxy and C₃-C₆-cycloalkyloxy,
R⁹ represents hydrogen, C₁-C₃-alkyl or C₃-C₆-cycloalkyl,
where C₂-C₃-alkyl may be substituted by a substituent, where the substituent is selected from the group consisting of hydroxyl, C₁-C₃-alkoxy and C₃-C₆-cycloalkyloxy,
R¹⁰ represents hydrogen, C₁-C₃-alkyl or C₃-C₆-cycloalkyl,
where C₂-C₃-alkyl may be substituted by a substituent, where the substituent is selected from the group consisting of hydroxyl, C₁-C₃-alkoxy and C₃-C₆-cycloalkyloxy,
R¹¹ represents hydrogen, hydroxyl, C₁-C₃-alkyl, C₁-C₃-alkoxy or C₃-C₆-cycloalkyloxy,
where alkyl and alkoxy may be substituted by a substituent, where the substituent is selected from the group consisting of hydroxyl, C₁-C₃-alkoxy and C₃-C₆-cycloalkyloxy,
R¹² represents hydrogen, C₁-C₃-alkyl or C₃-C₆-cycloalkyl,
where C₂-C₃-alkyl may be substituted by a substituent, where the substituent is selected from the group consisting of hydroxyl, C₁-C₃-alkoxy and C₃-C₆-cycloalkyloxy,
R² represents fluorine, chlorine, cyano, trifluoromethyl or trifluoromethoxy,
R³ represents hydrogen, chlorine, methyl, ethyl, n-propyl, methoxy, ethoxy or methoxymethyl,
or one of its salts, its solvates or the solvates of its salts.

2. Compound according to claim 1, characterized in that
R¹ represents a group of the formula 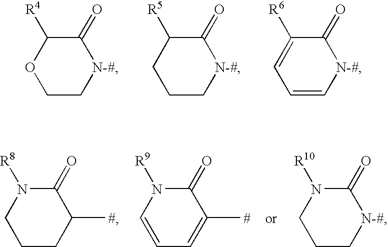
where
# is the point of attachment to the phenyl ring,
R⁴ represents hydrogen,
R⁵ represents hydrogen, hydroxyl, C₁-C₃-alkyl or C₁-C₃-alkoxy,
where alkyl and alkoxy may be substituted by a substituent, where the substituent is selected from the group consisting of hydroxyl and C₁-C₃-alkoxy,
R⁶ represents hydrogen, C₁-C₃-alkyl or C₁-C₃-alkoxy,
where alkyl and alkoxy may be substituted by a substituent, where the substituent is selected from the group consisting of hydroxyl and C₁-C₃-alkoxy,
R⁸ represents hydrogen, C₁-C₃-alkyl or C₃-C₆-cycloalkyl,
where C₂-C₃-alkyl may be substituted by a substituent, where the substituent is selected from the group consisting of hydroxyl and C₁-C₃-alkoxy,
R⁹ represents hydrogen, C₁-C₃-alkyl or C₃-C₆-cycloalkyl,
where C₂-C₃-alkyl may be substituted by a substituent, where the substituent is selected from the group consisting of hydroxyl and C₁-C₃-alkoxy,
R¹⁰ represents hydrogen, C₁-C₃-alkyl or C₃-C₆-cycloalkyl,
where C₂-C₃-alkyl may be substituted by a substituent, where the substituent is selected from the group consisting of hydroxyl and C₁-C₃-alkoxy,
R² represents fluorine or chlorine,
R³ represents hydrogen, methyl or methoxymethyl,
or one of its salts, its solvates or the solvates of its salts.

3. Compound according to claim 1 or 2, characterized in that
R¹ represents a group of the formula 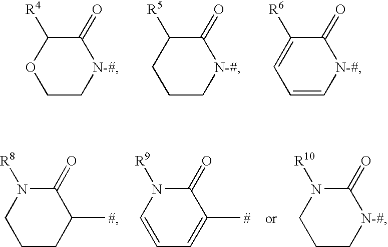
where
# is the point of attachment to the phenyl ring,
R⁴ represents hydrogen,
R⁵ represents hydrogen, hydroxyl or hydroxymethyl,
R⁶ represents hydrogen, methyl, hydroxymethyl, 2-hydroxyeth-1-yl or 2-hydroxyeth-1-oxy,
R⁸ represents hydrogen or methyl,
R⁹ represents hydrogen or methyl,
R¹⁰ represents methyl, ethyl or 2-hydroxyeth-1-yl,
R² represents fluorine or chlorine,
R³ represents hydrogen or methyl,
or one of its salts, its solvates or the solvates of its salts.

4. Compound according to any of claims 1 to 3, characterized in that
R¹ represents a group of the formula 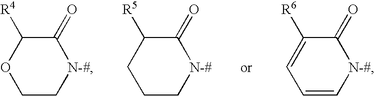
where
# is the point of attachment to the phenyl ring,
R⁴ is hydrogen,
R⁵ is hydrogen, hydroxyl or hydroxymethyl,
R⁶ is hydroxymethyl or 2-hydroxyeth-1-oxy,
R² is fluorine or chlorine,
R³ is hydrogen or methyl,
or one of its salts, its solvates or the solvates of its salts.

5. Process for preparing a compound of the formula (I) or one of its salts, its solvates or the solvates of its salts according to claim 1, characterized in that
[A] a compound of the formula 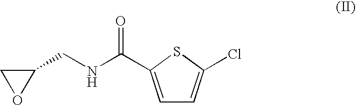
is, in the first step, reacted with a compound of the formula 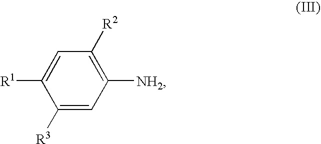
in which R¹, R² and R³ have the meaning given in claim 1,
to give a compound of the formula 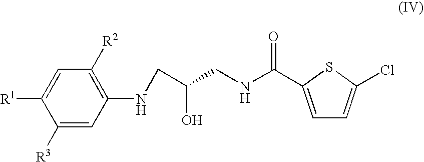
in which R¹, R² and R³ have the meaning given in claim 1,
and, in the second step, this compound is cyclised in the presence of phosgene or phosgene equivalents to give a compound of the formula (I),
or
[B] a compound of the formula 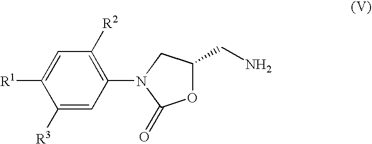
in which R¹, R² and R³ have the meaning given in claim 1
is reacted with a compound of the formula 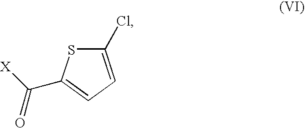
in which
X represents halogen, preferably bromine or chlorine, or hydroxyl.

6. Compound according to any of claims 1 to 4 for the treatment and/or prophylaxis of diseases.

7. Use of a compound according to any of claims 1 to 4 for preparing a medicament for the treatment and/or prophylaxis of diseases.

8. Use of a compound according to any of claims 1 to 4 for preparing a medicament for the treatment and/or prophylaxis of thromboembolic disorders.

9. Use of a compound according to any of claims 1 to 4 for preventing blood coagulation in vitro.

10. Medicament, comprising a compound according to any of claims 1 to 4 in combination with an inert nontoxic pharmaceutically acceptable auxiliary.

11. Medicament comprising a compound according to any of claims 1 to 4 in combination with a further active compound.

12. Medicament according to claim 10 or 11 for the treatment and/or prophylaxis of thromboembolic disorders.

13. Method for the treatment and/or prophylaxis of thromboembolic disorders in humans and animals using an anticoagulatory effective amount of at least one compound according to any of claims 1 to 4, a medicament according to any of claims 10 to 12 or a medicament obtained according to claim 7 or 8.

14. Method for preventing blood coagulation in vitro, characterized in that an anticoagulatory effective amount of a compound according to any of claims 1 to 4 is added.

15. Use of a compound according to any of claims 1 to 4 for preparing a medicament for the treatment and/or prophylaxis of pulmonary hypertension.

16. Use of a compound according to any of claims 1 to 4 for preparing a medicament for the treatment and/or prophylaxis of sepsis, systemic inflammatory syndrome (SIRS), septic organ dysfunction, septic organ failure and multiorgan failure, acute respiratory distress syndrome (ARDS), acute lung injury (ALI), septic shock, DIC (“disseminated intravascular coagulation”) and/or septic organ failure.

17. Compound, as defined in one of claims 1 to 4, for use in a method for the treatment and/or prophylaxis of thromboembolic disorders.
